(19) United States
(12) Patent Application Publication
(10) Pub. No.: US 2026/0041821 A1
(43) Pub. Date: Feb. 12, 2026

(54) DRUG-RELEASING COATING

(71) Applicant: Tonic Medical, Inc., Brooklyn Park, MN (US)

(72) Inventors: Lixiao Wang, Henderson, NV (US); Kasyap Venkata Seethamraju, Eden Prairie, MN (US); Peter Barnett, Shakopee, MN (US)

(21) Appl. No.: 19/350,447

(22) Filed: Oct. 6, 2025

Related U.S. Application Data
(63) Continuation-in-part of application No. 17/180,338, filed on Feb. 19, 2021.
(60) Provisional application No. 62/979,980, filed on Feb. 21, 2020.
(60) Provisional application No. 63/104,965, filed on Oct. 23, 2020.

(51) Int. Cl. A61L 29/08; A61L 29/16; A61M 25/10; C08L 5/08; C08L 67/04; C08L 79/02

(52) U.S. Cl. CPC A61L 29/085; A61L 29/16; A61M 25/1002; C08L 5/08; C08L 67/04; C08L 79/02; A61L 2420/06; A61L 2420/08

(57) ABSTRACT

A drug-releasing coating includes polymer-encapsulated drug particles including a therapeutic agent, one or more polymers that encapsulate the therapeutic agent, and a first ionic or zwitterionic additive. The first ionic or zwitterionic additive is in the polymer-encapsulated drug particles, coated on a surface of the polymer-encapsulated drug particles, or a combination thereof. The drug-releasing coating also includes a release matrix including a second ionic or zwitterionic additive.

### CROSS-REFERENCE TO RELATED APPLICATIONS

This application is a continuation-in-part of and claims the benefit of priority under 35 U.S.C. § 120 to U.S. Utility application Ser. No. 17/180,338 filed Feb. 19, 2021, which claims the benefit of priority to U.S. Provisional Patent Application Ser. No. 62/979,980 filed Feb. 21, 2020 and U.S. Provisional Patent Application Ser. No. 63/104,965 filed Oct. 23, 2020, the disclosures of which are incorporated herein in their entirety by reference.

The disclosure of each of the following applications are incorporated herein by reference in their entirety. U.S. patent application Ser. No. 16/135,436, which is a continuation-in-part of international Application No. PCT/US2018/031083 filed May 4, 2018, which claims the benefit of priority to U.S. Provisional Patent Application Ser. No. 62/502,212 filed May 5, 2017. U.S. patent application Ser. No. 16/135,436 is also a continuation-in-part of U.S. patent application Ser. No. 15/568,614 filed Oct. 23, 2017, which is a U.S. National Stage Filing under 35 U.S.C. 371 from International Application No. PCT/US2016/028652 filed Apr. 21, 2016, which claims the benefit of priority to U.S. Provisional Patent Application Ser. No. 62/152,559 filed Apr. 24, 2015. U.S. patent application Ser. No. 16/135,436 is also a continuation-in-part of U.S. patent application Ser. No. 14/438,327 filed Apr. 24, 2015, which is a U.S. National Stage Filing under 35 U.S.C. 371 from International Application No. PCT/US2013/064842 filed Oct. 14, 2013, which claims the benefit of priority to U.S. Provisional Patent Application Ser. No. 61/795,790 filed Oct. 26, 2012. U.S. patent application Ser. No. 16/135,472, which is a continuation-in-part of international Application No. PCT/US2018/031083 filed May 4, 2018, which claims the benefit of priority to U.S. Provisional Patent Application Ser. No. 62/502,212 filed May 5, 2017. U.S. patent application Ser. No. 16/135,472 is also a continuation-in-part of U.S. patent application Ser. No. 15/568,614 filed Oct. 23, 2017, which is a U.S. National Stage Filing under 35 U.S.C. 371 from International Application No. PCT/US2016/028652 filed Apr. 21, 2016, which claims the benefit of priority to U.S. Provisional Patent Application Ser. No. 62/152,559 filed Apr. 24, 2015. U.S. patent application Ser. No. 16/135,472 is also a continuation-in-part of U.S. patent application Ser. No. 14/438,327 filed Apr. 24, 2015, which is a U.S. National Stage Filing under 35 U.S.C. 371 from International Application No. PCT/US2013/064842 filed Oct. 14, 2013, which claims the benefit of priority to U.S. Provisional Patent Application Ser. No. 61/795,790 filed Oct. 26, 2012.

### BACKGROUND

Benign prostatic hyperplasia is a non-cancerous enlargement of the prostate gland, affecting more than 50% percent of men over the age of 60. The prostate early in life is the size and shape of a walnut and weighs about 20 grams. Prostate enlargement appears to be a normal process. With age, the prostate gradually increases in size to twice or more its normal size. As the prostate grows, it presses against and narrows the urethra, causing prostatic urethra compression and urinary obstruction that makes voiding difficult or impossible.

Male urethral stricture disease occurs at a rate as high as 0.6% in some populations. Urethral stricture diseases appear to be more common in the elderly population. Patients with urethral strictures experience moderate to severe complications, such as lower urinary tract voiding symptoms or urinary retention, recurrent urinary tract infection and the need for repeat urethral procedures such as dilation, urethrotomy, or urethroplasty.

Ureteral strictures of the upper urinary tract are either congenital or acquired. Congenital ureteral strictures are most commonly located at the ureteropelvic junction. Most ureteral strictures are acquired and usually are iatrogenic. The most common etiology of the ureteral strictures is injury during endoscopic, open, or laparoscopic surgical procedures.

Bladder neck strictures (e.g., stenosis or contracture) and urethral strictures are recognized complications of all treatments for prostate cancer. Recalcitrant bladder neck strictures are relatively rare overall; however, these are associated with significant morbidity, often requiring multiple interventions with associated complications and impact upon quality of life. Bladder neck strictures and urethral strictures are complications following treatment for prostate cancer such as radical prostatectomy (RP), radiotherapy, cryotherapy, and high intensity focused ultrasound (HIFU).

Strictures in the digestive body lumen or the gastrointestinal tracts include esophageal strictures, achalasia strictures, biliary strictures, stomach strictures, small intestine strictures, duodenum strictures, jejunum strictures, ileum strictures, colon strictures, rectum strictures, and large intestine strictures. The type of disease classifies a stricture into benign or malignant.

A biliary stricture, also referred to as a bile duct stricture, occurs when the bile duct gets smaller or narrower. The bile duct is the tube that takes bile from the liver to the small intestine. When the bile duct becomes narrow, it makes it difficult for food to digest. Biliary strictures can be caused by any injuries to the bile duct, swelling, pancreatitis, intestinal injuries, and cancers in the bile duct or pancreas. The symptoms of the biliary stricture include pain, chills and fever, itching, and nausea or vomiting.

Esophageal strictures are a problem commonly encountered in gastroenterological medicine and can be caused by malignant or benign lesions. Dysphagia is the symptom experienced by all patients. Most of these patients require palliative treatment to relieve the dysphagia.

Barrett's disease, also called Barrett's esophagus, is a condition in which there is an abnormal (metaplastic) change in the mucosal cells lining the lower portion of the esophagus, from normal stratified squamous epithelium to simple columnar epithelium with interspersed goblet cells that are normally present only in the colon. This change is considered to be a premalignant condition because it is associated with a high incidence of further transition to esophageal adenocarcinoma, an often-deadly cancer.

Eosinophilic esophagitis (EoE) is a chronic inflammatory disease. The symptoms of the disease include dysphagia and food impaction, and are often a consequence of esophageal strictures. Repeated endoscopic dilation of the esophageal fibrostenotic strictures of eosinophilic esophagitis using bougie and balloon catheter is used for treatment of such strictures.

Lower gastrointestinal tract strictures are a narrowing of a section of the intestine that causes problems by slowing or blocking the movement of food through the area. The strictures are caused by recurrent inflammations, cancer, Crohn's disease, and ulcerative colitis. The strictures include esophageal strictures, achalasia strictures, strictures in stents, biliary strictures, stomach strictures, small intestine strictures, duodenum strictures, jejunum strictures, ileum strictures, colon strictures, rectum strictures, and large intestine strictures.

Inflammatory bowel disease (IBD) includes Crohn's disease (CD) and ulcerative colitis (UC). Chrohn's disease- and ulcerative colitis-induced strictures are a common complication of inflammatory bowel disease and surgeries for the treatment thereof. Stricture rates for those suffering from inflammatory bowel disease range from 34% to 70% over time. Some of the strictures are refractory or reoccurring, which require repeated endoscopic dilation for treatment.

An anastomosis is a connection or opening between two body structures that carry fluid. A surgical anastomosis is the joining of two fluid-carrying body lumen structures via a surgical technique. An anastomotic stricture is a narrowing of an anastomosis. Anastomotic strictures are a common complication of surgical anastomoses and of various other surgical procedures such as radical prostatectomies, bowel resections, and gastric bypass surgery. Anastomotic strictures are usually fibrotic and can be difficult to manage and treat. An anastomotic stricture can include a stricture in an anastomosis between two portions of the same body structure, or between two different body structures, wherein the body structure can be an esophagus, biliary tract, stomach, small intestine, duodenum, jejunum, ileum, colon, rectum, large intestine, colon, rectum, urethra, ureter, or bladder neck. An anastomotic stricture can be a colorectal stricture, a stricture after gastric bypass, an ileocolonic stricture, a gastrointestinal stricture, a J-pouch stricture, or a bladder neck stricture (e.g., stenosis). While balloon dilation has been shown to be a safe and effective nonsurgical method of managing anastomotic strictures, problems still remain, such as a need for repeated balloon dilations due to refractory or reoccurring anastomotic strictures.

Vaginal stenosis is an abnormal condition in which the vagina becomes narrower and shorter due to the formation of fibrous tissue. Vaginal stenosis can have a negative impact on sexual dysfunction, dyspareunia and make pelvic exams difficult and painful. The lining of the vagina may also be thinner and drier and contain scar tissue. This condition can result in pain during sexual intercourse or a pelvic exam. Vaginal stenosis is often caused by an episiotomy, radiation therapy to the pelvis, or various types of surgery.

Radiation (e.g., radiotherapy) is used as one of mode of treatment for localized cancers. Localized cancer is the most commonly diagnosed cancer. The majority of patients are diagnosed in potentially curable early stages. Standard local treatment options include active surveillance, radical prostatectomy (RP) for prostate cancer, and, generally for all cancer treatments, radiotherapy (RT). Radiotherapy can be delivered via external beam (EBRT) or brachytherapy (BT). The side effects associated with each treatment can vary significantly. Localized cancers include prostate cancers, urethral cancers, ureteral cancers, esophageal cancers, biliary cancers, stomach cancers, small intestine cancers, duodenum cancers, jejunum cancers, ileum cancers, colon cancers, rectum cancers, large intestine cancers, and pulmonary cancers. The radiation treatment can create injury in adjacent health tissue, such as strictures. Radiation treatment-induced strictures can include urethral strictures, ureteral strictures, esophageal strictures, biliary tract strictures, stomach strictures, small intestine strictures, duodenum strictures, jejunum strictures, ileum strictures, colon strictures, rectum strictures, and large intestine strictures. Treatment of radiation-induced strictures can be complex and difficult. Due to the high survival rates, the number of prostate cancer survivors in the United States annually increased by 220,000 men up to almost 2.8 million in 2015, leaving a large number of men at risk for short- or long-term side effects of radiation therapy for prostate cancer treatment. The development of urethral strictures as a side effect of radiation therapy for prostate cancer treatment is particularly problematic.

Various minimally invasive methods used to treat various cancers, large colon polyps, and Barrett's esophagus are worldwide practice. The various minimally invasive methods are gaining favor over surgical procedures when the patients prefer to avoid a surgical operation. Several randomized controlled trials and meta-analyses have proven the clinical and oncological safety and effectiveness of the laparoscopic gastrectomy, robot-assisted gastrectomy, EMR (endoscopic mucosal resection) and ESD (endoscopic sub-mucosal dissection) in treatment of cancers and Barrett's esophagus of various stages. EMR (endoscopic mucosal resection) and ESD (endoscopic sub-mucosal dissection) are safe and effective for treatment of superficial cancers during the early stages, such as esophageal cancers, biliary cancers, stomach cancers, small intestine cancers, duodenum cancers, jejunum cancers, ileum cancers, colon cancers, rectum cancers, colorectal cancers, ileocolonic cancers, and gastrointestinal cancers. EMR and ESD are safe and effective for treatment of high-graded Barrett's esophagus. Laparoscopic gastrectomy, robot-assisted gastrectomy, EMR, and ESD are feasible procedures in terms of clinical and oncological safety; however, the recurrences of malignancy and refractory stricture were noted in some of patients. The local reoccurrence rate of cancers after minimally invasive treatments is in the range of 2-20%, depending on the type and stage of the cancer and on follow-up times. The occurrence rate of strictures or stenoses after minimally invasive procedures is about 26%-70%. Repeated endoscopic balloon dilations are needed to treat refractory or reoccurring strictures or stenoses.

Cardiovascular disease or atherosclerosis is characterized by hardening and narrowing of the arteries due to deposition of fatty plaques on the arterial lumens. Over time the plaque build-up can become severe enough to block the flow of oxygen rich blood to downstream tissue. Atherosclerosis can occur in any arteries in the body. The blockage, or stenosis, of critical arteries such as the carotid artery and coronary arteries can lead to sudden death. Stenosed peripheral arteries such as the iliac. superficial femoral, popliteal, tibial, and peroneal arteries can lead to the need for amputation. Chronic kidney disease can occur if the renal arteries are blocked. Currently drug-coated balloons and drug eluting stents are being used to treat the stenosis. Long term mortality rates, thrombosis rates, and efficacy of these drug coated devices is still not good enough. Better devices are needed to improve the safety and efficacy for cardiovascular disease stenosis.

Kidney failure is a disease that leads to the build-up of waste products in the blood. To prevent waste product build-up patients have a medical procedure called chronic hemodialysis. During hemodialysis the blood is sent through a dialyzer to remove waste products. The access point for the blood is typically in a limb at a special vascular junction created called an arteriovenous fistula (AVF) or arteriovenous grafts (AVG). AVFs are a special type of anastomosis in which the artery is sutured directly to a vein. AVGs are a different type of anastomosis in which the connection between the artery and vein is facilitated by a synthetic or autologous tube. AVFs and AVGs frequently become stenosed and cause blood flow obstruction. When this happens they must either be dilated open or abandoned for the creation of a new AVF or AVG in order to complete the dialysis treatments. Drug-coated balloons are used to dilate stenosed AVFs and AVGs but better devices are needed to improve safety and efficacy.

Heart valve disease is common disease afflicting the aging population. It is diagnosed by listening to the heartbeat with a stethoscope during a physical exam. An abnormal sound the heart makes is called a murmur and different murmurs can be indicative of a specific type of heart valve disease. Types of heart valve disease include stenosis, regurgitation, prolapse, and atresia. Heart valve stenosis is a narrowing or stiffening of the heart valve that results in the valve not opening or closing properly. The flaps of the valve may thicken, stiffen, or fuse together. As a result, the valve cannot fully open and the heart then has to work harder to pump blood through the valve. The result of this could be hypoxia or reduced oxygen supply affecting the whole body, local tissue, or a region of the body. Heart valve stenosis can be treated by a procedure called balloon valvuloplasty or BAV. BAV involves tracking an inflatable balloon through the vasculature to the heart valve and dilating the heart valve anulus. Recently the number of BAV procedures performed has increased because BAV is utilized before, after, and/or during transcatheter aortic valve replacement (TAVR) procedures. Restenosis rates following BAV have been noted in the 40-80% range at 5-9 months indicating the need for improvement in devices and technique.

### SUMMARY OF THE INVENTION

Various aspects of the present disclosure provide a drug-releasing coating. The drug-releasing coating includes polymer-encapsulated drug particles. The polymer-encapsulated drug particles include a therapeutic agent, one or more polymers that encapsulate the therapeutic agent, and a first ionic or zwitterionic additive. The first ionic or zwitterionic additive is in the polymer-encapsulated drug particles, coated on a surface of the polymer-encapsulated drug particles, or a combination thereof. The drug-releasing coating also includes a release matrix including a second ionic or zwitterionic additive.

Various aspects of the present disclosure provide a drug-releasing coating. The drug-releasing coating includes polymer-encapsulated drug particles that include sirolimus and PLGA polymer. The drug-releasing coating also includes a release matrix that includes polylysine and/or polyarginine in an amount that is 0.5% to 20% of a total weight of the polymer-encapsulated drug particles in the drug-releasing coating.

Various aspects of the present disclosure provide a drug-releasing coating. The drug-releasing coating includes polymer-encapsulated drug particles having a mean diameter (D50) of 0.5 μm to 5 μm and sirolimus drug content of 25 wt % to 65 wt %. The drug-releasing coating includes polylysine and/or polyarginine at 0.5% to 20% by weight of the polymer-encapsulated drug particles. The drug-releasing coating includes hyaluronic acid in a polylysine and/or polyarginine to hyaluronic acid ratio of 1:1 to 60:1. The drug-releasing coating also includes a topcoat layer including stearic acid 50, POPC, and BHT in a ratio of 2-7:1-4:1-2.

Various aspects of the present disclosure provide a drug-releasing coating. The drug-releasing coating includes polymer-encapsulated drug particles having a mean diameter (D50) of 0.5 μm to 5 μm and sirolimus drug content of 25 wt % to 65 wt %. The drug-releasing coating also includes POPC, DOPC, PEE, C6 or C7 fatty acid component, and BHT in a ratio of 0.5-2:0.01-0.3:0.1-0.5:0.01-0.3:0.5-2. The C6 fatty acid component is 1,2-dihexanoyl-sn-glycero-3-phosphocholine, and the C7 fatty acid component is 1,2-diheptanoyl-sn-glycero-3-phosphocholine. The POPC is present at 50% to 80% of a total weight of the of the polymer-encapsulated drug particles in the drug-releasing coating.

Various aspects of the present disclosure provide a drug-releasing coating. The drug-releasing coating includes polymer-encapsulated drug particles having a mean diameter (D50) of 0.5 μm to 5 μm and sirolimus drug content of 25 wt % to 65 wt %. The drug-releasing coating also includes POPC, C6 fatty acid component, and BHT in a ratio of 0.5-2:0.1-1:1-5. The C6 fatty acid component is 1,2-dihexanoyl-sn-glycero-3-phosphocholine. The POPC is present at 30% to 70% of a total weight of the polymer-encapsulated drug particles in the drug-releasing coating.

Various aspects of the present disclosure provide a balloon catheter. The balloon catheter includes an elongated balloon. The balloon catheter also includes a coating layer overlying an exterior surface of the balloon. The coating layer including a drug-releasing coating. The drug-releasing coating includes polymer-encapsulated drug particles. The polymer-encapsulated drug particles include a therapeutic agent, one or more polymers that encapsulate the therapeutic agent, and a first ionic or zwitterionic additive. The first ionic or zwitterionic additive is in the polymer-encapsulated drug particles, coated on a surface of the polymer-encapsulated drug particles, or a combination thereof. The drug-releasing coating also includes a release matrix including a second ionic or zwitterionic additive.

Various aspects of the present disclosure provide a method of making the drug-releasing coating of the present disclosure. The method includes dispersing polymer-encapsulated drug particles in water using ultrasonic mixing. The method includes adding a second ionic or zwitterionic additive to the dispersion. The method also includes applying the dispersion to a balloon catheter.

Various aspects of the present disclosure provide a method for treating a vascular condition. The method includes providing the balloon catheter of the present disclosure that includes a coating layer overlying an exterior surface of the balloon, wherein the coating layer includes the drug-releasing coating of the present disclosure. The method includes inserting the balloon catheter into a target site. The method includes inflating the balloon catheter to contact the coating layer with tissue at the target site. The method also includes deflating and removing the balloon catheter.

Various aspects of the present disclosure provide a method of making polymer-encapsulated drug particles. The method includes dissolving sirolimus, PLGA polymer, and a first ionic or zwitterionic additive in dichloromethane to form an oil phase. The method includes dispersing the oil phase in a water phase containing polyvinyl alcohol using high-pressure microfluidic apparatus. The method includes mixing the oil-water mixture with excess water for stabilization and solvent evaporation. The method includes centrifuging to separate microspheres. The method also includes lyophilizing to obtain dried microsphere powder.

Various aspects of the present disclosure provide a method of applying the drug-releasing coating of the present disclosure. The method includes dispersing polymer-encapsulated drug particles in water using ultrasonic mixing. The method includes adding polylysine and/or polyarginine in an amount that is 0.5% to 20% of a total weight of the polymer-encapsulated drug particles. The method optionally includes adding hyaluronic acid solution. The method includes applying the coating suspension to a balloon catheter targeting a dose density of the drug of 0.5 μg/mm² to 5 μg/mm². The method also includes applying a topcoat layer using cyclohexane or heptane as solvent.

Various aspects of the present disclosure provide a drug-releasing coating system. The system includes an aqueous base layer including polymer-encapsulated drug particles, polylysine and/or polyarginine, and optionally hyaluronic acid. The system also includes a non-aqueous topcoat layer including one or more phospholipids, one or more fatty acids, and one or more antioxidants dissolved in cyclohexane or heptane.

Various aspects of the drug-releasing coating of the present disclosure have certain advantages over other drug-releasing coatings or methods of making the same. For example, various aspects of the present disclosure specifically address limitations of conventional drug-releasing coating formulations and provide a superior solution that outperforms existing approaches. Advantages can include drug transfer and tissue retention advantages, coating durability and performance advantages, surface chemistry and adhesion advantages, manufacturing and regulatory advantages, storage and stability advantages, clinical performance advantages, device design and procedural advantages, formulation flexibility advantages, airway treatment advantages, or a combination thereof.

Various aspects of the drug-releasing coating and method of making the same of the present disclosure have various drug transfer and tissue retention advantages over conventional drug-releasing coatings. For example, various aspects of the drug-releasing coating and method of making the same of the present disclosure achieve higher drug transfer efficiency compared to other drug-releasing coatings. Various aspects of the drug-releasing coating and method of making the same of the present disclosure transfer greater percentage of drug to tissue during a given inflation time compared to other drug-releasing coatings. Various aspects of the drug-releasing coating and method of making the same of the present disclosure provide higher tissue adhesion/retention rates of the drug compared to other drug-releasing coatings. Various aspects of the drug-releasing coating composition and method of making the same of the present disclosure outperform conventional drug-releasing coatings by delivering enhanced drug retention by treated tissues with drug remaining in tissue after overnight agitation under physiological conditions and providing rapid drug elution and superior permeation of drug into tissues at target site with enhanced rate and extent of absorption of antiproliferative therapeutic agent in airway strictures or stenosis.

Various aspects of the present disclosure provide various coating durability and performance advantages over other drug-releasing coatings or methods of making the same. For example, various aspects of the drug-releasing coating and method of making the same of the present disclosure maintain enhanced coating durability compared to other drug-releasing coatings. Various aspects of the drug-releasing coating and method of making the same of the present disclosure achieve enhanced performance compared to other drug-releasing coatings. Various aspects of the drug-releasing coating and method of making the same of the present disclosure retain significantly more drug after advancement through tortuous vessel models in-vitro compared to other drug-releasing coatings while delivering superior coating integrity during device tracking through anatomy.

Various aspects of the present disclosure provide various surface chemistry and adhesion advantages over other drug-releasing coatings or methods of making the same. For example, various aspects of the drug-releasing coating of the present disclosure provide enhanced surface charge properties compared to other drug-releasing coatings while maintaining more strongly positive zeta potential than other drug-releasing coatings. Various aspects of the drug-releasing coating composition of the present disclosure have strongly positive zeta potentials correlating with improved adhesion to various tissue surfaces that other approaches struggle to achieve.

Various aspects of the present disclosure provide improved controlled release and clinical performance advantages over other drug-releasing coatings or methods of making the same. Various aspects of the drug-releasing coating or method of making the same of the present disclosure provide comparable or superior tissue drug levels over time compared to other drug-releasing coatings. Various aspects of the drug-releasing coating composition or method of making the same of the present disclosure provide higher drug levels in tissue over time compared to other drug-releasing coatings with similar amounts of therapeutic agent in the coating. Various aspects of the drug-releasing coating and method of making the same of the present disclosure demonstrate effectiveness over greater variety of applications than other drug-releasing coatings with efficacy for vascular, urological, and gastrointestinal applications while delivering improved clinical outcomes and lower systemic exposure to the therapeutic agent.

Various aspects of the present disclosure provide manufacturing and regulatory advantages over other drug-releasing coatings or methods of making the same. Various aspects of the drug-releasing coating or method of making the same of the present disclosure enable higher drug loading capability and higher degree of dose density control than other drug-releasing coatings. Various aspects of the drug-releasing coating composition or method of making the same of the present disclosure provide better maintenance of drug retention properties and tissue adhesion properties after electron beam sterilization compared to other sterilized drug-releasing coatings. Various aspects of the drug-releasing coating and method of making the same of the present disclosure demonstrate more compatibility with large-scale manufacturing with more highly reproducible drug transfer and retention profiles compared to other drug-releasing coatings while supporting wider range of additive combinations and solvent compatibility than other drug-releasing coatings, enabling tailored coatings for various clinical applications.

Various aspects of the present disclosure provide storage and stability advantages over other drug-releasing coatings or methods of making the same. Various aspects of the drug-releasing coating or method of making the same of the present disclosure enable decreased leakage of therapeutic agent during manufacturing and long-term storage of medical device. Various aspects of the drug-releasing coating composition or method of making the same of the present disclosure provide various ways to decrease or minimize leakage of therapeutic agent outside of polymer-encapsulated drug particles that other formulations lack.

Various aspects of the present disclosure provide device design and procedural advantages over other drug-releasing coatings or methods of making the same. For example, various aspects of the drug-releasing coating of the present disclosure demonstrate balloon catheter design with instrument access port for improved navigation, integration with positioning instruments for enhanced procedural control, compatibility with image-guided surgery systems through sensor integration, and flexible balloon sizing options for various anatomical applications. Various aspects of the drug-releasing coating composition of the present disclosure have dual lumen design with integrated inflation lumen and stiffening member for enhanced device performance that differentiate the technology from prior art formulations.

Various aspects of the present disclosure provide formulation flexibility advantages over other drug-releasing coatings or methods of making the same. Various aspects of the drug-releasing coating or method of making the same of the present disclosure enable ability to incorporate various ionic and zwitterionic additives for optimized performance and compatibility with different polymer systems for encapsulation. Various aspects of the drug-releasing coating composition or method of making the same of the present disclosure provide flexibility in particle size optimization, incorporation of antioxidants for enhanced stability, and use of phospholipid components for improved biocompatibility that other approaches struggle to achieve while delivering manufacturing versatility that other formulations lack.

Various aspects of the present disclosure provide specific airway treatment advantages over other drug-releasing coatings or methods of making the same. For example, various aspects of the drug-releasing coating and method of making the same of the present disclosure provide delivery of antiproliferative therapeutic agent to airway body lumen during very brief deployment time compared to conventional approaches. Various aspects of the drug-releasing coating and method of making the same of the present disclosure achieve reduction of re-narrowing and reoccurring of strictures of nonvascular body lumen while facilitating rapid drug elution and superior permeation of drug into tissues at target site.

Various aspects of the present disclosure provide airway stenosis and stricture treatment advantages over other drug-releasing coatings or methods of making the same. Various aspects of the drug-releasing coating or method of making the same of the present disclosure enable treatment of recurring airway stricture or stenosis, laryngostenosis, subglottic stricture or stenosis, and benign airway stenosis with superior clinical outcomes. Various aspects of the drug-releasing coating composition or method of making the same of the present disclosure provide freedom from symptom-driven target lesion reintervention and improvement in forced expiratory volume in one second in patients post-procedure. Various aspects of the drug-releasing coating and method of making the same of the present disclosure demonstrate improvement in modified Medical Research Council dyspnea score and improvement in Peak Expiratory Flow in patients at post-procedure timepoints.

Various aspects of the present disclosure provide chronic rhinosinusitis treatment advantages over other drug-releasing coatings or methods of making the same. For example, various aspects of the drug-releasing coating of the present disclosure provide freedom from target lesion reintervention due to recurrence of chronic rhinosinusitis or chronic rhinosinusitis with nasal polyps post-procedure. Various aspects of the drug-releasing coating composition of the present disclosure have mean change from baseline in sino-nasal outcome test score and mean change from baseline in Lund-Mackay computed tomography score at post-procedure timepoints. Various aspects of the drug-releasing coating and method of making the same of the present disclosure demonstrate therapeutic agent measurable in blood of fewer patients at any time point following treatment with therapeutic agent not being measurable in blood of any patients by post-treatment timepoints.

Various aspects of the present disclosure provide severe asthma treatment advantages over other drug-releasing coatings or methods of making the same. Various aspects of the drug-releasing coating or method of making the same of the present disclosure enable reduction of exacerbation of severe asthma and reducing concentration of bronchial smooth muscle cells of severe asthma patients. Various aspects of the drug-releasing coating composition or method of making the same of the present disclosure provide reducing concentration of eosinophils, interleukin-4, interleukin-5, interleukin-6, interleukin-13, interleukin-23, and innate lymphoid cell type 2 in airway tract while achieving improvement in asthma quality of life questionnaire score from baseline at post-procedure timepoints. Various aspects of the drug-releasing coating and method of making the same of the present disclosure deliver reduction in asthma control questionnaire score from baseline, improvement in asthma control test score from baseline, and reduction in annualized rate of severe exacerbations.

Various aspects of the present disclosure provide respiratory condition treatment advantages over other drug-releasing coatings or methods of making the same. Various aspects of the drug-releasing coating or method of making the same of the present disclosure enable treatment of mucuous plug in airway, reduction of concentration of goblet cells in airway tract, and reducing concentration of one or more mucins in the airway tract. Various aspects of the drug-releasing coating composition or method of making the same of the present disclosure provide treatment of chronic obstructive pulmonary disease, chronic bronchitis, bronchial fibrosis, and treating central airway obstruction with enhanced therapeutic outcomes compared to conventional approaches. Various aspects of the drug-releasing coating and method of making the same of the present disclosure demonstrate capability for treatment of airway tumors in human subjects while maintaining safety profile and effectiveness across diverse respiratory pathology applications.

Various aspects of the present disclosure provide nasal and sinus access advantages over other drug-releasing coatings or methods of making the same. Various aspects of the drug-releasing coating or method of making the same of the present disclosure enable treatment across diverse anatomical locations including frontal sinus, ethmoid sinus, sphenoid sinus, maxillary sinus, nasal passage, supraglottis, glottis, subglottis, trachea, mainstem bronchus, bronchus intermedius, and lobar bronchus. Various aspects of the drug-releasing coating composition or method of making the same of the present disclosure provide capability for treatment of specific bronchial anatomical locations including right primary bronchus, right upper lobe bronchus, right lower lobe bronchus, multiple segmental bronchi, left upper lobe bronchus, left lower lobe bronchus, and multiple left-sided bronchial segments with precise anatomical targeting that other approaches cannot achieve.

### BRIEF DESCRIPTION OF THE FIGURES

The drawings illustrate generally, by way of example, but not by way of limitation, various aspects of the present invention.

FIG. 1 is a perspective view of an aspect of a balloon catheter according to the present invention (the balloon catheter includes a fixed wire, over the wire, and rapid exchanged balloon catheters details not shown in FIG. 1), in accordance with various aspects.

FIGS. 2A-2C are cross-sectional views of different aspects of the distal portion of the balloon catheter of FIG. 1 at line A-A, showing exemplary coating layers, in accordance with various aspects.

FIG. 3A illustrates a balloon catheter having one neck section, in accordance with various aspects.

FIG. 3B illustrates a balloon catheter having two neck sections, in accordance with various aspects.

FIG. 3C illustrates a balloon catheter having three neck sections, in accordance with various aspects.

FIG. 4A-4D illustrate a balloon catheter that includes an elongated rigid member, in accordance with various aspects.

FIG. 5 illustrates an elongated rigid member that is a spring, in accordance with various aspects.

FIG. 6 is a diagram and table showing an example of drug coating particle size analysis using a Beckman Coulter LS 13 320 Particle Sizing Analyzer with Liquid Analyzer Module, in accordance with various aspects.

FIGS. 7A-7C illustrate SEM images of an example of a sirolimus coated balloon, with FIG. 7A showing 37×, FIG. 7B showing 1,600×, and FIG. 7C showing 7,500×, in accordance with various aspects.

FIGS. 8A-8C illustrate diagrams of exemplary powder x-ray diffraction graphs obtained from: FIG. 8A crystalline sirolimus, FIG. 8B dodecyl glycerol, and FIG. 8C sterilized sirolimus drug coating on balloon.

FIGS. 9A-9C illustrate diagrams of DSC scans of: FIG. 9A crystalline sirolimus, FIG. 9B dodecyl glycerol, and FIG. 9C sirolimus drug-coated balloon, in accordance with various aspects.

FIG. 10 illustrates a diagram of the sirolimus particle size reduction obtained with a high-pressure homogenizer, in accordance with various aspects.

FIG. 11 illustrates a freedom from reintervention Kaplan-Meier curve for paclitaxel-coated balloon treatment in esophagus and bowel, in accordance with various aspects.

FIG. 12 illustrates drug residuals for Examples 12, 29, 19, and 28, in accordance with various aspects.

FIG. 13 illustrates 1 day and 7 day pk for Examples 12, 29, 19, and 28, in accordance with various aspects.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “polymer” refers to a molecule having at least one repeating unit and can include copolymers.

### Drug-Releasing Coating.

Various aspects of the present disclosure provide a drug-releasing coating. The drug-releasing coating includes polymer-encapsulated drug particles. The polymer-encapsulated drug particles can include a therapeutic agent. The polymer-encapsulated drug particles can include one or more polymers that encapsulate the therapeutic agent. The polymer-encapsulated drug particles can include a first ionic or zwitterionic additive. The first ionic or zwitterionic additive can be in the polymer-encapsulated drug particles, coated on a surface of the polymer-encapsulated drug particles, or a combination thereof. The drug-releasing coating can also include a release matrix including a second ionic or zwitterionic additive. The release matrix can include the polymer-encapsulated drug particles homogeneously distributed therein.

The polymer-encapsulated drug particle can have any suitable zeta potential, such as a negative zeta potential, or a positive zeta potential. The zeta potential is the electrical potential at the slipping plane (i.e., the at the interface which separates mobile fluid from fluid that remains attached to the surface of the particle). The zeta potential of the polymer-encapsulated drug particle can be measured in any suitable way, such as using electrophoretic light scattering (ELS) or electroacoustic determination. The polymer-encapsulated drug particle can have a positive zeta potential, such as a zeta potential of greater than zero, or 1-50, or 2-40, or less than or equal to 50 and greater than or equal to 1 and less than, equal to, or greater than 2, 3, 4, 5, 6, 8, 10, 12, 14, 16, 18, 20, 25, 30, 35, 40, or 45. The polymer-encapsulated drug particle can have a negative zeta potential, such as a zeta potential or less than zero, or −1 to −50, or −2 to −40, or more positive than or equal to −50 and less positive than or equal to −1 and less than, equal to, or greater −45, −40, −35, −30, −25, −20, −18, −16, −14, −12, −10, −8, −6, −5, −4, −3, or −2.

The therapeutic agent in the polymer-encapsulated drug particle can be any suitable therapeutic agent. The therapeutic agent can include paclitaxel, docetaxel, taxol, an mTOR inhibitor, rapamycin, sirolimus, zotarolimus, everolimus, tacrolimus, umirolimus, an analogue thereof, and combinations thereof. The therapeutic agent can be sirolimus. The therapeutic agent can be crystalline, partially crystalline, amorphous, partially amorphous, or a combination thereof. The therapeutic agent can be crystalline and/or partially crystalline. The therapeutic agent can have any suitable largest dimension (e.g., largest diameter), such as a mean largest dimension of 0.1 to 29.9 microns, or 0.5 to 15 microns, or 1 to 10 microns, 0.5 microns to 5 microns, or less than or equal to 29.9 microns and greater than or equal to 0.1 micron, 0.5, 1, 2, 3, 4, 5, 6, 7, 8, 9, 10, 12, 14, 16, 18, 20, 22, 24, 26, 28, or 29 microns. The therapeutic agent can form any suitable proportion of the polymer-encapsulated drug particle, such as 1-80 wt %, or 10-80 wt %, or 5-45 wt %, or 25-65 wt %, or 25-35 wt %, or less than or equal to 80 wt % and equal to or greater 1 wt % and less than, equal to, or greater than 5, 10, 15, 20, 25, 26, 27, 28, 29, 30, 31, 32, 33, 34, 35, 40, 45, 50, 55, 60, 65, 70, or 75 wt %. Particle diameters can be measured in any suitable way, such as via laser diffraction analysis.

The polymer in the polymer-encapsulated drug particles that encapsulates the therapeutic agent can be any suitable polymer. The polymer can include one or more polymers chosen from polylactic acid (PL), polyglycolic acid (GA), a polylactic acid/polyglycolic acid copolymer (PLGA), polydioxanone, polycaprolactone, polyphosphazene, collagen, gelatin, chitosan, glycosoaminoglycans, and copolymers thereof. The polymer can include PLGA. As used herein, “encapsulate” can refer to 50-100% surface area coverage of the encapsulated material (i.e., therapeutic agent and optionally the first ionic or zwitterionic additive) by the encapsulant (e.g., polymer), or 60-100%, 75-100%, 90-100%, or equal to or greater than 50% and less than or equal to 100% and less than, equal to, or greater than 55%, 60, 65, 70, 75, 80, 85, 90, 92, 94, 96, 98, 99, 99.5, or 99.9%. The polymer can be any suitable proportion of the polymer-encapsulated drug particles, such as 10-95 wt %, or 30-80 wt %, or 50-75 wt %, or less than or equal to 95 wt % and equal to or greater than 10 wt % and less than, equal to, or greater than 15 wt %, 20, 25, 30, 35, 40, 45, 50, 55, 56, 57, 58, 59, 60, 61, 62, 63, 64, 65, 66, 67, 68, 69, 70, 75, 80, 85, or 90 wt %.

The polymer-encapsulated drug particles can have any suitable mean largest dimension or mean diameter (D50), such as 0.1 μm to 10 μm, 0.5 μm to 5 μm, or less than or equal to 10 μm and greater than or equal to 0.1 μm and greater than, equal to, or less than 0.2 μm, 0.3, 0.4, 0.5, 0.6, 0.7, 0.8, 0.9, 1, 1.2, 1.4, 1.6, 1.8, 2, 2.2, 2.4, 2.6, 2.8, 3, 3.5, 4, 4.5, 5, 5.5, 6, 6.5, 7, 7.5, 8, 8.5, 9, or 9.5 μm. Particle diameters can be measured in any suitable way, such as via laser diffraction analysis.

The first ionic or zwitterionic additive can be in the polymer-encapsulated drug particles. The first ionic or zwitterionic additive can be coated on a surface of the polymer-encapsulated drug particles. The first ionic or zwitterionic additive can be in the polymer-encapsulated drug particles and coated on a surface of the polymer-encapsulated drug particles. The first ionic or zwitterionic additive can include an ionic additive, a zwitterionic additive, or a combination thereof. The first ionic or zwitterionic additive can include a charged polymer, a charged lipid, a phospholipid, a phosphocholine, a phosphatidylcholine, a phosphatidylethanolamine, a phosphatidylserine, a phosphatidylinositol, or a combination thereof. The first ionic or zwitterionic additive can include 1,2-dipalmitoyl-sn-glycero-3-ethylphosphocholine (chloride salt), 1,2-distearoyl-sn-glycero-3-ethylphosphocholine (chloride salt), cholic acid, deoxycholic acid, chenodeoxycholic acid, lithocholic acid, 1,2-dilauroyl-sn-glycero-3-phosphoglycerol, sodium salt, 1,2-dihexanoyl-sn-glycero-3-phosphocholine, 1,2-diheptanoyl-sn-glycero-3-phosphocholine, 1,2-dioctanoyl-sn-glycero-3-phosphocholine, 1,2-dinonanoyl-sn-glycero-3-phosphocholine, 1,2-didecanoyl-sn-glycero-3-phosphocholine, 1,2-diundecanoyl-sn-glycero-3-phosphocholine, 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1-palmitoyl-2-stearoyl-sn-glycero-3-phosphocholine, 1-stearoyl-2-palmitoyl-sn-glycero-3-phosphocholine, 1-palmitoyl-2-lauroyl-sn-glycero-3-phosphocholine, 1,2-dioleoyl-sn-glycero-3-phosphocholine (DOPC), 1-palmitoyl-2-oleoyl-glycero-3-phosphocholine (POPC), 1-stearoyl-2-hydroxy-sn-glycero-3-phosphocholine, 1-lauroyl-2-hydroxy-sn-glycero-3-phosphocholine, 1-myristoyl-2-hydroxy-sn-glycero-3-phosphocholine, 1-palmitoyl-2-hydroxy-sn-glycero-3-phosphocholine, dieicosenoyl phosphatidylcholine (1,2-dieicosenoyl-sn-glycero-3-phosphocholine, C20:1 PC), diarachidonoyl phosphatidylcholine (1,2-diarachidoyl-sn-glycero-3-phosphocholine, C20:0 PC), dierucoyl phosphatidylcholine (1,2-dierucoyl-sn-glycero-3-phosphocholine, C22:1 PC), didocosahexaenoyl phosphatidylcholine (1,2-didocosahexaenoyl-sn-glycero-3-phosphocholine, C22:6 PC), heneicosenoyl phosphatidylcholine (1,2-heneicosenoyl-sn-glycero-3-phosphocholine, C21:1 PC), dinervonyl phosphatidylcholine (1,2-dinervonoyl-sn-glycero-3-phosphocholine, C24:1 PC), 1,2-disteroyl-sn-glycero-3-phosphatidylcholine (DSPC), or a combination thereof. The first ionic or zwitterionic additive can include 1,2-disteroyl-sn-glycero-3-phosphatidylcholine (DSPC). The first ionic or zwitterionic additive can be any suitable proportion of the polymer-encapsulated drug particles. The first ionic or zwitterionic additive can be 0.01 wt % to 50 wt % of the polymer-encapsulated drug particles, or 0.01 wt % to 20 wt %, or 0.1 wt % to 5 wt %, or 0.5 wt % to 2 wt %, or less than or equal to 50 wt % and greater than or equal to 0.01 wt % and less than, equal to, or greater than 0.05 wt %, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 5, 6, 7, 8, 9, 10, 11, 12, 13, 14, 15, 16, 17, 18, 19, 20, 22, 24, 26, 28, 30, 32, 34, 36, 38, 40, 42, 44, 46, or 48 wt % of the polymer-encapsulated drug particles.

The polymer-encapsulated drug particles can include a fatty acid component. The fatty acid component can be any suitable fatty acid component. The fatty acid component can include a C6-C20 fatty acid component that is C6-C20 fatty acid esterified to a glycero-3-phosphocholine as a 1,2-di(C6-C20 fatty acid ester)-sn-glycero-3-phosphocholine. The C6-C20 fatty acid esterified to the glycerol-3-phosphocholine can be any suitable C6-C20 fatty acid, such as a C6 fatty acid (e.g., caproic acid (hexanoic acid): C6:0), a C7 fatty acid (e.g., heptanoic acid: C7:0), a C8 fatty acid (e.g., caprylic acid (octanoic acid): C8:0), a C9 fatty acid (e.g., pelargonic acid (nonanoic acid): C9:0), a C10 fatty acid (e.g., capric acid (decanoic acid): C10:0), a C11 fatty acid (e.g. undecylic acid (undecanoic acid): C11:0), a C12 fatty acid (e.g., lauric acid (dodecanoic acid): C12:0), a C13 fatty acid (e.g., tridecylic acid (tridecanoic acid): C13:0), a C14 fatty acid (e.g., myristic acid (tetradecanoic acid): C14:0, or myristoleic acid: C14:1), a C15 fatty acid (e.g., pentadecanoic acid (pentadecylic acid): C15:0), a C16 fatty acid (e.g., palmitic acid (hexadecanoic acid): C16:0, palmitoleic acid: C16:1, or sapienic acid: C16:1), or a C17 fatty acid (e.g., margaric acid (heptadecanoic acid): C17:0, or heptadecenoic acid: C17:1). The at least one fatty acid component can include a C6 fatty acid component such as 1,2-dihexanoyl-sn-glycero-3-phosphocholine. The at least one fatty acid component include a C7 fatty acid component such as 1,2-diheptanoyl-sn-glycero-3-phosphocholine. The at least one fatty acid component can be chosen from stearic acid 50, a C6 fatty acid component, a C7 fatty acid component, and combinations thereof. The at least one fatty acid component can include stearic acid 50 (e.g., a blend of stearic and palmitic acids). The at least one fatty acid component can be any suitable proportion of the polymer-encapsulated drug particles, such as 0.01 wt % to 30 wt %, or 0.1 wt % to 10 wt %, or less than or equal to 30 wt % and greater than or equal to 0.01% and less than, equal to, or greater than 0.05%, 0.1, 0.2, 0.4, 0.6, 0.8, 1, 1.5, 2, 2.5, 3, 4, 5, 6, 8, 10, 12, 14, 16, 18, 20, 22, 24, 26, or 28 wt %.

The polymer encapsulated drug-particles can include an antioxidant. The antioxidant can be any suitable antioxidant. The antioxidant can be BHT. The antioxidant can be any suitable proportion of the polymer-encapsulated drug-particles, such as 0.01 wt % to 30 wt %, or 0.1 wt % to 10 wt %, or less than or equal to 30 wt % and greater than or equal to 0.01% and less than, equal to, or greater than 0.05%, 0.1, 0.2, 0.4, 0.6, 0.8, 1, 1.5, 2, 2.5, 3, 4, 5, 6, 8, 10, 12, 14, 16, 18, 20, 22, 24, 26, or 28 wt %.

The polymer-encapsulated drug particles can include a phospholipid (e.g., DSPC), a fatty acid component (e.g., a C6 or C7 fatty acid component, such as dihexanoyl-sn-glycero-3-phosphocholine or 1,2-diheptanoyl-sn-glycero-3-phosphocholine), an antioxidant (e.g., BHT), or a combination thereof.

The polymer-encapsulated drug particles can be any suitable proportion of the drug-releasing coating, such as 1 wt % to 95 wt % of the drug-releasing coating, or 25 wt % to 65 wt %, or less than or equal to 95 wt % and greater than or equal to 1 wt % and less than, equal to, or greater than 2 wt %, 3, 4, 5, 6, 8, 10, 12, 14, 16, 18, 20, 25, 30, 35, 40, 45, 50, 55, 60, 65, 70, 75, 80, 85, or 90 wt %. The polymer-encapsulated drug particles can be homogenously distributed in the release matrix.

The second ionic or zwitterionic additive can include a charged polymer, a charged lipid, a phospholipid, a phosphocholine, a phosphatidylcholine, a phosphatidylethanolamine, a phosphatidylserine, a phosphatidylinositol, or a combination thereof. The second ionic or zwitterionic additive can include 1,2-dipalmitoyl-sn-glycero-3-ethylphosphocholine (chloride salt), 1,2-distearoyl-sn-glycero-3-ethylphosphocholine (chloride salt), cholic acid, deoxycholic acid, chenodeoxycholic acid, lithocholic acid, 1,2-dilauroyl-sn-glycero-3-phosphoglycerol, sodium salt, 1,2-dihexanoyl-sn-glycero-3-phosphocholine, 1,2-diheptanoyl-sn-glycero-3-phosphocholine, 1,2-dioctanoyl-sn-glycero-3-phosphocholine, 1,2-dinonanoyl-sn-glycero-3-phosphocholine, 1,2-didecanoyl-sn-glycero-3-phosphocholine, 1,2-diundecanoyl-sn-glycero-3-phosphocholine, 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1-palmitoyl-2-stearoyl-sn-glycero-3-phosphocholine, 1-stearoyl-2-palmitoyl-sn-glycero-3-phosphocholine, 1-palmitoyl-2-lauroyl-sn-glycero-3-phosphocholine, 1,2-dioleoyl-sn-glycero-3-phosphocholine (DOPC), 1-palmitoyl-2-oleoyl-glycero-3-phosphocholine (POPC), 1-stearoyl-2-hydroxy-sn-glycero-3-phosphocholine, 1-lauroyl-2-hydroxy-sn-glycero-3-phosphocholine, 1-myristoyl-2-hydroxy-sn-glycero-3-phosphocholine, 1-palmitoyl-2-hydroxy-sn-glycero-3-phosphocholine, dieicosenoyl phosphatidylcholine (1,2-dieicosenoyl-sn-glycero-3-phosphocholine, C20:1 PC), diarachidonoyl phosphatidylcholine (1,2-diarachidoyl-sn-glycero-3-phosphocholine, C20:0 PC), dierucoyl phosphatidylcholine (1,2-dierucoyl-sn-glycero-3-phosphocholine, C22:1 PC), didocosahexaenoyl phosphatidylcholine (1,2-didocosahexaenoyl-sn-glycero-3-phosphocholine, C22:6 PC), heneicosenoyl phosphatidylcholine (1,2-heneicosenoyl-sn-glycero-3-phosphocholine, C21:1 PC), dinervonyl phosphatidylcholine (1,2-dinervonoyl-sn-glycero-3-phosphocholine, C24:1 PC), 1,2-disteroyl-sn-glycero-3-phosphatidylcholine (DSPC), 1-palmitoyl-2-oleoyl-sn-glycero-3-phosphocholine (POPC), 1,2-dioleoyl-sn-glycero-3-phosphocholine (DOPC), 1,2-dipalmitoleoyl-sn-glycero-3-phosphocholine (DPEPC), 1,2-disteroyl-sn-glycero-3-phosphatidylcholine (DSPC), polylysine, polyarginine, hyaluronic acid (HA), or a combination thereof. The second ionic or zwitterionic additive can include 1-palmitoyl-2-oleoyl-sn-glycero-3-phosphocholine (POPC), 1,2-dioleoyl-sn-glycero-3-phosphocholine (DOPC), 1,2-dipalmitoleoyl-sn-glycero-3-phosphocholine (DPEPC), 1,2-disteroyl-sn-glycero-3-phosphatidylcholine (DSPC), polylysine, polyarginine, hyaluronic acid (HA), or a combination thereof. The second ionic or zwitterionic additive can be any suitable proportion of the drug-releasing coating, such as 5 wt % to 99 wt % of the drug-releasing coating, or 5 wt % to 65 wt %, or less than or equal to 99 wt % and greater than or equal to 5 wt % and less than, equal to, or greater than 10 wt %, 15, 20, 25, 30, 35, 40, 45, 50, 55, 60, 65, 70, 75, 80, 85, 90, 95, or 98 wt %. The second ionic or zwitterionic additive can be present in the drug-releasing coating in an amount that is 0.01 wt % to 200 wt % of a total amount of the polymer-encapsulated drug particles in the drug-releasing coating, or 1 wt % to 150 wt %, or less than or equal to 200 wt % and greater than or equal to 0.01 wt % and less than, equal to, or greater than 0.05 wt %, 0.1, 0.5, 1, 1.5, 2, 2.5, 3, 4, 5, 6, 8, 10, 12, 14, 16, 18, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 195 wt % of a total amount of the polymer-encapsulated drug particles in the drug-releasing coating.

The second ionic or zwitterionic additive can include a cationic polymer. The cationic polymer can include polyethylenimine (PEI), polyallylamine, polypropylenimine, polyamidoamine dendrimer, cationic polyoxazoline, poly(beta-aminoester), PEG-PEI copolymer, PLGA-PEI copolymer, positively charged gelatin (e.g., base-treated gelatin), hydroxy-terminated poly(2-methyl-2-oxazoline), poly(2-ethyl-2-oxazoline), stearic acid-modified branched polyethylenimine, branched PEI-g-PEG, poly(1-vinylpyrrolidone-co-2-dimethylaminoethyl methacrylate), poly(1-vinylpyrrolidone)-graft-(1-triacontene), poly-L-lysine, poly-L-ornithine, poly(4-hydroxy-L-proline ester), polylysine, polyarginine, poly(N,N-dimethylaminoethyl methacrylate), cationic copolymer of dimethylaminoethyl methacrylate/butyl methacrylate/methyl methacrylate (e.g., Eudragit E), polycation-containing cyclodextrin, amino cyclodextrin or a derivative thereof, amino dextran, histone, protamine, cationized human serum albumin, aminopolysaccharide, chitosan, a peptide, polylysine, polyarginine, or a combination thereof. The cationic polymer can include polylysine. The cationic polymer can include polyarginine. The cationic polymer can include a combination of polylysine and polyarginine. The release matrix can include the cationic polymer in an amount that is 0.1% to 40% of a total weight of the polymer-encapsulated drug particles in the drug-releasing coating, or 0.5% to 20%, or less than or equal to 40% and greater than or equal to 0.1% and less than, equal to, or greater than 0.5%, 1, 1.5, 2, 2.5, 3, 3.5, 4, 4.5, 5, 6, 7, 8, 9, 10, 11, 12, 13, 14, 15, 16, 17, 18, 19, 20, 22, 24, 26, 28, 30, 32, 34, 36, or 38% of a total weight of the polymer-encapsulated drug particles in the drug-releasing coating.

The second ionic or zwitterionic additive can include an anionic polymer. The release matrix can include the anionic polymer in an amount that is 0.01% to 30%, or 0.1% to 10% of a total weight of the polymer-encapsulated drug particles in the drug-releasing coating, or less than or equal to 30% and greater than or equal to 0.01% and less than, equal to, or greater than 0.1, 1, 1.5, 2, 2.5, 3, 3.5, 4, 4.5, 5, 6, 7, 8, 9, 10, 11, 12, 13, 14, 15, 16, 17, 18, 19, 20, 22, 24, 26, 28, or 30% of a total weight of the polymer-encapsulated drug particles in the drug-releasing coating. The anionic polymer can include any suitable anionic polymer. The anionic polymer can include hyaluronic acid (HA).

The second ionic or zwitterionic additive can include a cationic polymer and an anionic polymer that are present in a weight ratio ranging from 1:1 to 60:1, or 2:1 to 30:1. Or less than or equal to 60:1 and greater than or equal to 1:1 and less than, equal to, or greater than 1.2:1, 1.4:1, 1.6:1, 1.8:1, 2:1, 2.5:1, 3:1, 3.5:1, 4:1, 4.5:1, 5:1, 6:1, 7:1, 8:1, 9:1, 10:1, 12:1, 14:1, 16:1, 18:1, 20:1, 22:1, 24:1, 26:1, or 28:1.

The release matrix can include an antioxidant. The antioxidant can be present in the release matrix in an amount that is 0.1% to 150% of a total weight of the polymer-encapsulated drug particles in the drug-releasing coating, or 1% to 100%, or less than or equal to 150% and greater than or equal to 0.1% and less than, equal to, or greater than 0.5%, 1, 2, 3, 4, 5, 6, 8, 10, 12, 14, 16, 18, 20, 25, 30, 35, 40, 45, 50, 55, 60, 65, 70, 75, 80, 85, 90, 95, 100, 105, 110, 115, 120, 125, 130, 135, 140, or 145% of a total weight of the polymer-encapsulated drug particles in the drug-releasing coating. The antioxidant can be any suitable antioxidant. The antioxidant can include butylated hydroxytoluene (BHT).

The release matrix can include a fatty acid component. The fatty acid component can be any suitable fatty acid component. The fatty acid component can include a C6-C20 fatty acid component that is C6-C20 fatty acid esterified to a glycero-3-phosphocholine as a 1,2-di(C6-C20 fatty acid ester)-sn-glycero-3-phosphocholine. The C6-C20 fatty acid esterified to the glycerol-3-phosphocholine can be any suitable C6-C20 fatty acid, such as a C6 fatty acid (e.g., caproic acid (hexanoic acid): C6:0), a C7 fatty acid (e.g., heptanoic acid: C7:0), a C8 fatty acid (e.g., caprylic acid (octanoic acid): C8:0), a C9 fatty acid (e.g., pelargonic acid (nonanoic acid): C9:0), a C10 fatty acid (e.g., capric acid (decanoic acid): C10:0), a C11 fatty acid (e.g. undecylic acid (undecanoic acid): C11:0), a C12 fatty acid (e.g., lauric acid (dodecanoic acid): C12:0), a C13 fatty acid (e.g., tridecylic acid (tridecanoic acid): C13:0), a C14 fatty acid (e.g., myristic acid (tetradecanoic acid): C14:0, or myristoleic acid: C14:1), a C15 fatty acid (e.g., pentadecanoic acid (pentadecylic acid): C15:0), a C16 fatty acid (e.g., palmitic acid (hexadecanoic acid): C16:0, palmitoleic acid: C16:1, or sapienic acid: C16:1), or a C17 fatty acid (e.g., margaric acid (heptadecanoic acid): C17:0, or heptadecenoic acid: C17:1). The at least one fatty acid component include a C6 fatty acid component such as 1,2-dihexanoyl-sn-glycero-3-phosphocholine. The at least one fatty acid component can include a C7 fatty acid component such as 1,2-diheptanoyl-sn-glycero-3-phosphocholine. The at least one fatty acid component can be chosen from stearic acid 50, a C6 fatty acid component, a C7 fatty acid component, and combinations thereof. The at least one fatty acid component can include stearic acid 50 (e.g., a blend of stearic and palmitic acids). The at least one fatty acid component can be any suitable proportion of the release matrix, such as 0.01 wt % to 30 wt %, or 0.1 wt % to 10 wt %, or less than or equal to 30 wt % and greater than or equal to 0.01% and less than, equal to, or greater than 0.05%, 0.1, 0.2, 0.4, 0.6, 0.8, 1, 1.5, 2, 2.5, 3, 4, 5, 6, 8, 10, 12, 14, 16, 18, 20, 22, 24, 26, or 28 wt %. The at least one fatty acid component in the release matrix can be 1% to 200% of the weight of the polymer-encapsulated drug particles in the drug-releasing coating, or less than or equal to 200% and greater than or equal to 1% and less than, equal to, or greater than 2%, 3, 4, 5, 6, 7, 8, 9, 10, 12, 14, 16, 18, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, or 195% of the weight of the polymer-encapsulated drug particles in the drug-releasing coating.

The release matrix can include pentaerythritol or a pentaerythritol ether, such as pentaerythritol ethoxylate (PEE) (e.g., 15/4 EO/OH, or 3/4 EO/OH), pentaerythritol propoxylate, pentaerythritol propoxylate/ethoxylate, glycerol ethoxylate, glycerol propoxylate, trimethylolpropane ethoxylate, dipentaerythritol, or a combination thereof. The pentaerythritol or a pentaerythritol ether can be any suitable proportion of the release matrix, such as 1% to 30% of the weight of the polymer-encapsulated drug particles in the drug-releasing coating, or 5% to 15%, or less than or equal to 30% and greater than or equal to 1% and less than, equal to, or greater than 2%, 3, 4, 5, 6, 7, 8, 9, 10, 11, 12, 13, 14, 15, 16, 17, 18, 19, 20, 21, 22, 23, 24, 25, 26, 27, 28, or 29% of the weight of the polymer-encapsulated drug particles in the drug-releasing coating.

The release matrix can include a phospholipid (e.g., 1-palmitoyl-2-oleoyl-sn-glycero-3-phosphocholine (POPC), 1,2-dioleoyl-sn-glycero-3-phosphocholine (DOPC), 1,2-dipalmitoleoyl-sn-glycero-3-phosphocholine (DPEPC), 1,2-disteroyl-sn-glycero-3-phosphatidylcholine (DSPC)), a cationic polymer (e.g., polylysine and/or polyarginine), an anionic polymer (e.g., hyaluronic acid), a fatty acid component (e.g., a C6 or C7 fatty acid component, such as dihexanoyl-sn-glycero-3-phosphocholine or 1,2-diheptanoyl-sn-glycero-3-phosphocholine), an antioxidant (e.g., BHT), or a combination thereof.

In various aspects, the release matrix can include POPC, DOPC, pentaerythritol ethoxylate (PEE), a C6 or C7 fatty acid component (e.g., dihexanoyl-sn-glycero-3-phosphocholine or 1,2-diheptanoyl-sn-glycero-3-phosphocholine), and BHT. The release matrix can include POPC, DOPC, PEE, a C6 or C7 fatty acid component (e.g., dihexanoyl-sn-glycero-3-phosphocholine or 1,2-diheptanoyl-sn-glycero-3-phosphocholine), and BHT in a weight ratio of 0.5-2 (e.g., less than or equal to 2 and greater than or equal to 0.5 and less than, equal to, or greater than 0.6, 0.7, 0.8, 0.9, 1, 1.1, 1.2, 1.3, 1.4, 1.5, 1.6, 1.7, 1.8, or 1.9):0.01-0.3 (e.g., less than or equal to 0.3 and greater than or equal to 0.01 and less than, equal to, or greater than 0.02, 0.04, 0.06, 0.08, 0.1, 0.12, 0.14, 0.16, 0.18, 0.2, 0.22, 0.24, 0.26, or 0.28):0.1-0.5 (e.g., less than or equal to 0.5 and greater than or equal to 0.1 and less than, equal to, or greater than 0.15, 0.2, 0.25, 0.3, 0.35, 0.4, or 0.45):0.01-0.3 (e.g., less than or equal to 0.3 and greater than or equal to 0.01 and less than, equal to, or greater than 0.02, 0.04, 0.06, 0.08, 0.1, 0.12, 0.14, 0.16, 0.18, 0.2, 0.22, 0.24, 0.26, or 0.28):0.5-2 (e.g., less than or equal to 2 and greater than or equal to 0.5 and less than, equal to, or greater than 0.6, 0.7, 0.8, 0.9, 1, 1.1, 1.2, 1.3, 1.4, 1.5, 1.6, 1.7, 1.8, or 1.9). For example, the ratio can be about 1:0.1:0.2:0.1:1.

In various aspects, the release matrix can include POPC, a C6 or C7 fatty acid component (e.g., dihexanoyl-sn-glycero-3-phosphocholine or 1,2-diheptanoyl-sn-glycero-3-phosphocholine), and BHT. The release matrix can include POPC, a C6 or C7 fatty acid component (e.g., dihexanoyl-sn-glycero-3-phosphocholine or 1,2-diheptanoyl-sn-glycero-3-phosphocholine), and BHT in a weight ratio of 0.5-2 (e.g., less than or equal to 2 and greater than or equal to 0.5 and less than, equal to, or greater than 0.6, 0.7, 0.8, 0.9, 1, 1.1, 1.2, 1.3, 1.4, 1.5, 1.6, 1.7, 1.8, or 1.9):0.1-1 (e.g., less than or equal to 1 and greater than or equal to 0.1 and less than, equal to, or greater than 0.2, 0.3, 0.4, 0.5, 0.6, 0.7, 0.8, or 0.9):1-5 (e.g., less than or equal to 5 and greater than or equal to 1 and less than, equal to, or greater than 1.5, 2, 2.5, 3, 3.5, 4, or 4.5). For example, the ratio can be about 1:0.2:2.13.

The drug-releasing coating can further include a topcoat layer. The topcoat layer can be on top of the layer including the polymer-encapsulated drug particles and the release matrix. A substrate on which the drug-releasing coating is applied can be adjacent to the layer including the polymer-encapsulated drug particles and the release layer, such that the substrate and the topcoat layer sandwich the layer including the polymer-encapsulated drug particles and the release layer.

The topcoat layer can include a third ionic or zwitterionic additive. The third ionic or zwitterionic additive can include a charged polymer, a charged lipid, a phospholipid, a phosphocholine, a phosphatidylcholine, a phosphatidylethanolamine, a phosphatidylserine, a phosphatidylinositol, or a combination thereof. The third ionic or zwitterionic additive can include 1,2-dipalmitoyl-sn-glycero-3-ethylphosphocholine (chloride salt), 1,2-distearoyl-sn-glycero-3-ethylphosphocholine (chloride salt), cholic acid, deoxycholic acid, chenodeoxycholic acid, lithocholic acid, 1,2-dilauroyl-sn-glycero-3-phosphoglycerol, sodium salt, 1,2-dihexanoyl-sn-glycero-3-phosphocholine, 1,2-diheptanoyl-sn-glycero-3-phosphocholine, 1,2-dioctanoyl-sn-glycero-3-phosphocholine, 1,2-dinonanoyl-sn-glycero-3-phosphocholine, 1,2-didecanoyl-sn-glycero-3-phosphocholine, 1,2-diundecanoyl-sn-glycero-3-phosphocholine, 1,2-dilauroyl-sn-glycero-3-phosphocholine, 1,2-dimyristoyl-sn-glycero-3-phosphocholine, 1,2-dipalmitoyl-sn-glycero-3-phosphocholine, 1,2-distearoyl-sn-glycero-3-phosphocholine, 1-palmitoyl-2-stearoyl-sn-glycero-3-phosphocholine, 1-stearoyl-2-palmitoyl-sn-glycero-3-phosphocholine, 1-palmitoyl-2-lauroyl-sn-glycero-3-phosphocholine, 1,2-dioleoyl-sn-glycero-3-phosphocholine (DOPC), 1-palmitoyl-2-oleoyl-glycero-3-phosphocholine (POPC), 1-stearoyl-2-hydroxy-sn-glycero-3-phosphocholine, 1-lauroyl-2-hydroxy-sn-glycero-3-phosphocholine, 1-myristoyl-2-hydroxy-sn-glycero-3-phosphocholine, 1-palmitoyl-2-hydroxy-sn-glycero-3-phosphocholine, dieicosenoyl phosphatidylcholine (1,2-dieicosenoyl-sn-glycero-3-phosphocholine, C20:1 PC), diarachidonoyl phosphatidylcholine (1,2-diarachidoyl-sn-glycero-3-phosphocholine, C20:0 PC), dierucoyl phosphatidylcholine (1,2-dierucoyl-sn-glycero-3-phosphocholine, C22:1 PC), didocosahexaenoyl phosphatidylcholine (1,2-didocosahexaenoyl-sn-glycero-3-phosphocholine, C22:6 PC), heneicosenoyl phosphatidylcholine (1,2-heneicosenoyl-sn-glycero-3-phosphocholine, C21:1 PC), dinervonyl phosphatidylcholine (1,2-dinervonoyl-sn-glycero-3-phosphocholine, C24:1 PC), 1,2-disteroyl-sn-glycero-3-phosphatidylcholine (DSPC), 1-palmitoyl-2-oleoyl-sn-glycero-3-phosphocholine (POPC), 1,2-dioleoyl-sn-glycero-3-phosphocholine (DOPC), 1,2-dipalmitoleoyl-sn-glycero-3-phosphocholine (DPEPC), 1,2-disteroyl-sn-glycero-3-phosphatidylcholine (DSPC), 1,2-dilauroyl-sn-glycero-3-phosphocholine (DLPC), 1,2-dipalmitoleoyl-sn-glycero-3-phosphocholine (DPEPC), 1,2-disteroyl-sn-glycero-3-phosphatidylcholine (DSPC), steric acid, palmitic acid, hexanoic acid, heptanoic acid, or a combination thereof, polylysine, polyarginine, hyaluronic acid (HA), or a combination thereof. The third ionic or zwitterionic additive can include 1-palmitoyl-2-oleoyl-sn-glycero-3-phosphocholine (POPC), 1,2-dioleoyl-sn-glycero-3-phosphocholine (DOPC), 1,2-dilauroyl-sn-glycero-3-phosphocholine (DLPC), 1,2-dipalmitoleoyl-sn-glycero-3-phosphocholine (DPEPC), 1,2-disteroyl-sn-glycero-3-phosphatidylcholine (DSPC), steric acid, palmitic acid, hexanoic acid, heptanoic acid, or a combination thereof. The third ionic or zwitterionic additive can be 10 wt % to 100 wt % of the topcoat layer, or 65 wt % to 95 wt %, or less than or equal to 100 wt % and greater than or equal to 10 wt % and less than, equal to, or greater than 15 wt %, 20, 25, 30, 35, 40, 45, 50, 55, 60, 65, 70, 75, 80, 85, or 90 wt % of the topcoat layer. The third ionic or zwitterionic additive can be present in the topcoat layer in an amount that is 1% to 200% of a total weight of the polymer-encapsulated drug particles in the drug-releasing coating, or 3% to 150%, or less than or equal to 200% and greater than or equal to 1% and less than, equal to, or greater than 2%, 3, 4, 5, 6, 8, 10, 12, 14, 16, 18, 20, 25, 30, 35, 40, 45, 50, 55, 60, 65, 70, 75, 80, 85, 90, 95, 100, 105, 110, 115, 120, 125, 130, 135, 140, or 145% of a total weight of the polymer-encapsulated drug particles in the drug-releasing coating.

The topcoat layer can include a phospholipid (e.g. POPC, DOPC, DLPC, DPEPC, DSPC, or a combination thereof), a fatty acid component (e.g., a C6 or C7 fatty acid component, such as dihexanoyl-sn-glycero-3-phosphocholine or 1,2-diheptanoyl-sn-glycero-3-phosphocholine), an antioxidant (e.g., BHT), or a combination thereof.

The third ionic or zwitterionic additive can include at least one phospholipid. The at least one phospholipid can be chosen from 1-palmitoyl-2-oleoyl-sn-glycero-3-phosphocholine (POPC), 1,2-dioleoyl-sn-glycero-3-phosphocholine (DOPC), 1,2-dilauroyl-sn-glycero-3-phosphocholine (DLPC), 1,2-dipalmitoleoyl-sn-glycero-3-phosphocholine (DPEPC), 1,2-disteroyl-sn-glycero-3-phosphatidylcholine (DSPC), and combinations thereof. The at least one phospholipid can include 1-palmitoyl-2-oleoyl-sn-glycero-3-phosphocholine (POPC). The at least one phospholipid can include 1,2-dioleoyl-sn-glycero-3-phosphocholine (DOPC). The at least one phospholipid can include 1,2-dilauroyl-sn-glycero-3-phosphocholine (DLPC). The at least one phospholipid can include 1,2-dipalmitoleoyl-sn-glycero-3-phosphocholine (DPEPC). The at least one phospholipid can include a phosphatidylethanolamine. The topcoat layer can include the one or more phospholipids in an amount that is 1% to 150% of a total weight of the polymer-encapsulated drug particles in the drug-releasing coating, or 3% to 140%, or less than or equal to 150% and greater than or equal to 1% and less than, equal to, or greater than 2%, 3, 4, 5, 6, 8, 10, 12, 14, 16, 18, 20, 25, 30, 35, 40, 45, 50, 55, 60, 65, 70, 75, 80, 85, 90, 95, 100, 105, 110, 115, 120, 125, 130, 135, 140, or 145% of a total weight of the polymer-encapsulated drug particles in the drug-releasing coating.

The third ionic or zwitterionic additive can include at least one fatty acid component. The fatty acid component can be any suitable fatty acid component. The fatty acid component can include a C6-C20 fatty acid component that is C6-C20 fatty acid esterified to a glycero-3-phosphocholine as a 1,2-di(C6-C20 fatty acid ester)-sn-glycero-3-phosphocholine. The C6-C20 fatty acid esterified to the glycerol-3-phosphocholine can be any suitable C6-C20 fatty acid, such as a C6 fatty acid (e.g., caproic acid (hexanoic acid): C6:0), a C7 fatty acid (e.g., heptanoic acid: C7:0), a C8 fatty acid (e.g., caprylic acid (octanoic acid): C8:0), a C9 fatty acid (e.g., pelargonic acid (nonanoic acid): C9:0), a C10 fatty acid (e.g., capric acid (decanoic acid): C10:0), a C11 fatty acid (e.g. undecylic acid (undecanoic acid): C11:0), a C12 fatty acid (e.g., lauric acid (dodecanoic acid): C12:0), a C13 fatty acid (e.g., tridecylic acid (tridecanoic acid): C13:0), a C14 fatty acid (e.g., myristic acid (tetradecanoic acid): C14:0, or myristoleic acid: C14:1), a C15 fatty acid (e.g., pentadecanoic acid (pentadecylic acid): C15:0), a C16 fatty acid (e.g., palmitic acid (hexadecanoic acid): C16:0, palmitoleic acid: C16:1, or sapienic acid: C16:1), or a C17 fatty acid (e.g., margaric acid (heptadecanoic acid): C17:0, or heptadecenoic acid: C17:1). The at least one fatty acid component include a C6 fatty acid component such as 1,2-dihexanoyl-sn-glycero-3-phosphocholine. The at least one fatty acid component can include a C7 fatty acid component such as 1,2-diheptanoyl-sn-glycero-3-phosphocholine. The at least one fatty acid component can be chosen from stearic acid 50, a C6 fatty acid component, a C7 fatty acid component, and combinations thereof. The at least one fatty acid component can include stearic acid 50 (e.g., a blend of stearic and palmitic acids). The topcoat layer can include the one or more fatty acid components in an amount that is 1% to 30% of a total weight of the polymer-encapsulated drug particles in the drug-releasing coating, or 2% to 20%, or less than or equal to 30% and greater than or equal to 1% and less than, equal to, or greater than 2%, 3, 4, 5, 6, 7, 8, 9, 10, 11, 12, 13, 14, 15, 16, 17, 18, 19, 20, 22, 24, 26, or 28% of a total weight of the polymer-encapsulated drug particles in the drug-releasing coating.

The topcoat layer can include an antioxidant. The antioxidant can be any suitable antioxidant. The antioxidant can be BHT. The antioxidant can be present in the topcoat in an amount that is 0.1% to 120% of a total weight of the polymer-encapsulated drug particles in the drug-releasing coating, or 1% to 20%, or less than or equal to 120% and greater than or equal to 0.1% and less than, equal to, or greater than 1%, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, or 115%.

The topcoat can include pentaerythritol or a pentaerythritol ether, such as pentaerythritol ethoxylate (PEE) (e.g., 15/4 EO/OH, or 3/4 EO/OH), pentaerythritol propoxylate, pentaerythritol propoxylate/ethoxylate, glycerol ethoxylate, glycerol propoxylate, trimethylolpropane ethoxylate, dipentaerythritol, or a combination thereof. The pentaerythritol or a pentaerythritol ether can be any suitable proportion of the topcoat, such as 1% to 30% of the weight of the polymer-encapsulated drug particles in the drug-releasing coating, or 5% to 15%, or less than or equal to 30% and greater than or equal to 1% and less than, equal to, or greater than 2%, 3, 4, 5, 6, 7, 8, 9, 10, 11, 12, 13, 14, 15, 16, 17, 18, 19, 20, 21, 22, 23, 24, 25, 26, 27, 28, or 29% of the weight of the polymer-encapsulated drug particles in the drug-releasing coating.

The topcoat can include neat drug particles. The neat drug particles can be formed of any drug described herein as suitable for the therapeutic agent in the polymer-encapsulated drug particles, such as paclitaxel, docetaxel, taxol, an mTOR inhibitor, rapamycin, sirolimus, zotarolimus, everolimus, tacrolimus, umirolimus, an analogue thereof, and combinations thereof. The neat drug particles can be formed of the same drug as the therapeutic agent in the polymer-encapsulated drug particles. The neat drug particles can be sirolimus. The neat drug particles can have any suitable average particle size (D50), such as 0.5 μm to 10 μm, or 1 μm to 3 μm, or less than or equal to 10 μm and greater than or equal to 0.5 μm and less than, equal to, or greater than 1 μm, 1.5, 2, 2.5, 3, 4, 5, 6, 7, 8, or 9 μm.

In various aspects, the topcoat layer includes POPC and BHT in a weight ratio of 0.1:1 to 10:1, or 0.5:1 to 2:1, or less than or equal to 10:1 and greater than or equal to 0.1:1 and less than, equal to, or greater than 0.1:1, 0.2:1, 0.3:1, 0.4:1, 0.5:1, 0.6:1, 0.7:1, 0.8:1, 0.9:1, 1:1, 1.2:1, 1.4:1, 1.6:1, 1.8:1, 2:1, 2.5:1, 3:1, 3.5:1, 4:1, 5:1, 6:1, 7:1, 8:1, or 9:1.

In various aspects, the topcoat layer can include stearic acid 50 and POPC in a weight ratio ranging from 1:1 to 20:1, or less than or equal to 20:1 and greater than or equal to 1:1 and less than, equal to, or greater than 1.2:1, 1.4:1, 1.6:1, 1.8:1, 2:1, 2.5:1, 3:1, 4:1, 5:1, 6:1, 7:1, 8:1, 9:1, 10:1, 12:1, 14:1, 16:1, or 18:1.

In various aspects, the topcoat layer can include stearic acid 50, POPC, and BHT in a weight ratio of 1-10 (e.g., less than or equal to 10 and greater than or equal to 1 and less than, equal to, or greater than 2, 3, 4, 5, 6, 7, 8, or 9):0.5-8 (e.g., less than or equal to 8 and greater than or equal to 0.5 and less than, equal to, or greater than 1, 1.5, 2, 2.5, 3, 4, 5, 6, or 7):0.5-4 (e.g., less than or equal to 4 and greater than or equal to 0.5 and less than, equal to, or greater than 1, 1.5, 2, 2.5, 3, or 3.5). For example, the topcoat layer can include stearic acid 50, POPC, and BHT in a weight ratio of 2-7:1-4:1-2, such as in a ratio of 2:1:1, 4:2:1, or 6.7:3.3:1.5.

In various aspects, the topcoat layer can include POPC, DOPC, DLPC, C6 (e.g., dihexanoyl-sn-glycero-3-phosphocholine), and BHT in a weight ratio of 0.5-2 (e.g., less than or equal to 2 and greater than or equal to 0.5 and less than, equal to, or greater than 0.6, 0.7, 0.8, 0.9, 1, 1.1, 1.2, 1.3, 1.4, 1.5, 1.6, 1.7, 1.8, or 1.9):0.5-2 (e.g., less than or equal to 2 and greater than or equal to 0.5 and less than, equal to, or greater than 0.6, 0.7, 0.8, 0.9, 1, 1.1, 1.2, 1.3, 1.4, 1.5, 1.6, 1.7, 1.8, or 1.9):0.1-0.5 (e.g., less than or equal to 0.5 and greater than or equal to 0.1 and less than, equal to, or greater than 0.2, 0.3, or 0.4):1-3 (e.g., less than or equal to 3 and greater than or equal to 1 and less than, equal to, or greater than 1.2, 1.4, 1.6, 1.8, 2, 2.2, 2.4, 2.6, or 2.8):1-3 (e.g., less than or equal to 3 and greater than or equal to 1 and less than, equal to, or greater than 1.2, 1.4, 1.6, 1.8, 2, 2.2, 2.4, 2.6, or 2.8). For example, the ratio can be 1:1:0.26:1.73:1.71.

In various aspects, the topcoat layer can include POPC, DPEPC, BHT, and neat drug in a weight ratio of 1-2 (e.g., less than or equal to 2 and greater than or equal to 1 and less than, equal to, or greater than 1.2, 1.4, 1.6, or 1.8):1-5 (e.g., less than or equal to 5 and greater than or equal to 1 and less than, equal to, or greater than 1.5, 2, 2.5, 3, 3.5, 4, or 4.5):5-15 (e.g., less than or equal to 15 and greater than or equal to 5 and less than, equal to, or greater than 6, 7, 8, 9, 10, 11, 12, 13, or 14):20-40 (e.g., less than or equal to 40 and greater than or equal to 20 and less than, equal to, or greater than 21, 22, 23, 24, 25, 26, 27, 28, 29, 30, 31, 32, 33, 34, 35, 36, 37, 38, or 39). For example, the ratio can be 1.4:3.2:12.3:27.3.

The topcoat layer can be any suitable proportion of the drug-releasing coating. In various aspects, the topcoat layer can be present in an amount that is 1% to 90% of a total weight of the polymer-encapsulated drug particles in the drug-releasing coating, or 5% to 65%, or less than or equal to 90% and greater than or equal to 1% and less than, equal to, or greater than 2, 3, 4, 5, 6, 8, 10, 12, 14, 16, 18, 20, 25, 30, 35, 40, 45, 50, 55, 60, 65, 70, 75, 80, or 85% of a total weight of the polymer-encapsulated drug particles in the drug-releasing coating.

In various aspects, the drug-releasing coating can include polymer-encapsulated drug particles including sirolimus and PLGA polymer. The drug-releasing coating can further include a release matrix including polylysine and/or polyarginine in an amount that is 0.5% to 20% of a total weight of the polymer-encapsulated drug particles in the drug-releasing coating. In various aspects, the drug-releasing coating can further include hyaluronic acid in a polylysine and/or polyarginine to hyaluronic acid ratio of 1:1 to 60:1. In various aspects, the drug-releasing coating can further include a topcoat layer including stearic acid 50 and POPC.

In various aspects, the drug-releasing coating can include polymer-encapsulated drug particles having a mean diameter (D50) of 0.5 μm to 5 μm and sirolimus drug content of 25 wt % to 65 wt %. The drug releasing coating can further include a release matrix including polylysine and/or polyarginine at 0.5% to 20% by weight of the polymer-encapsulated drug particles. The release matrix can further include hyaluronic acid in a polylysine and/or polyarginine to hyaluronic acid ratio of 1:1 to 60:1. The drug-releasing coating can further include a topcoat layer including stearic acid 50, POPC, and BHT in a ratio of 2-7:1-4:1-2.

In various aspects, the drug-releasing coating can include polymer-encapsulated drug particles having a mean diameter (D50) of 0.5 μm to 5 μm and sirolimus drug content of 25 wt % to 65 wt %. The drug-releasing coating can further include a release matrix that includes POPC, DOPC, PEE, C6 or C7 fatty acid component (e.g., dihexanoyl-sn-glycero-3-phosphocholine or 1,2-diheptanoyl-sn-glycero-3-phosphocholine), and BHT in a ratio of 0.5-2:0.01-0.3:0.1-0.5:0.01-0.3:0.5-2. The POPC can be present at 50% to 80% of a total weight of the of the polymer-encapsulated drug particles in the drug-releasing coating. In various aspects, the drug-releasing coating is applied to achieve a target dose density of 0.5 μg/mm² to 5 μg/mm².

In various aspects, the drug-releasing coating can include polymer-encapsulated drug particles having a mean diameter (D50) of 0.5 μm to 5 μm and sirolimus drug content of 25 wt % to 65 wt %. The drug releasing coating can include POPC, C6 fatty acid component (e.g., dihexanoyl-sn-glycero-3-phosphocholine), and BHT in a ratio of 0.5-2:0.1-1:1-5. The POPC can be present at 30% to 70% of a total weight of the polymer-encapsulated drug particles in the drug-releasing coating. In various aspects, the drug-releasing coating can be applied to achieve a target dose density of 0.5 μg/mm² to 5 μg/mm².

The drug-releasing coating can be located on any suitable surface intended for drug delivery to a target location, such as on a medical device, such as on a balloon catheter, a drug-coated catheter, a drug-eluting stent, a drug-eluting stent on a balloon, a drug-eluting stent on a drug-coated balloon, a stent on a drug-coated balloon, or a combination thereof.

In various aspects, the present disclosure provides a drug-releasing coating system. The coating system can include an aqueous base layer including polymer-encapsulated drug particles, polylysine and/or polyarginine, and optionally hyaluronic acid. The coating system can include a non-aqueous topcoat layer including one or more phospholipids, one or more fatty acids, and one or more antioxidants dissolved in cyclohexane or heptane. In various aspects, the base layer and topcoat layer can provide controlled drug release and enhanced tissue adhesion through cationic charge. In various aspects, the coating system is designed to provide improved retention during catheter tracking through tortuous anatomy.

### Method of Making Drug-Releasing Coating.

Various aspects of the present disclosure provide a method of making the drug-releasing coating of the present disclosure. The method can include dispersing polymer-encapsulated drug particles in water using ultrasonic mixing. The method can include adding a second ionic or zwitterionic additive to the dispersion. The method can also include applying the dispersion to a balloon catheter.

The method can further include applying a topcoat layer including at least one phospholipid. The topcoat layer can be applied using cyclohexane as a solvent. The topcoat layer can be applied using heptane as a solvent.

The method can include sterilizing the coated balloon catheter using electron beam radiation. The electron beam radiation can be applied at a dose of 10 kGy to 100 kGy, or 15 kGy to 40 kGy, or less than or equal to 100 kGy and greater than or equal to 10 kGy and less than, equal to, or greater than 15 kGy, 20, 25, 30, 35, 40, 45, 50, 55, 60, 65, 70, 75, 80, 85, 90, or 95 kGy.

### Method of Applying a Drug-Releasing Coating.

Various aspects of the present disclosure provide a method of applying the drug-releasing coating of the present disclosure. The method can include dispersing polymer-encapsulated drug particles in water using ultrasonic mixing. The method can include adding polylysine and/or polyarginine in an amount that is 0.5% to 20% of a total weight of the polymer-encapsulated drug particles. The method can optionally include adding hyaluronic acid solution. The method can include applying the coating suspension to a medical device targeting a dose density of the drug of 0.5 μg/mm² to 5 μg/mm². The method can include applying a topcoat layer using cyclohexane or heptane as solvent. In various aspects, the ultrasonic mixing is performed using an ultrasonic mixing horn. The medical device can be any suitable medical device. The medical device can be a balloon catheter, a drug-coated catheter, a drug-eluting stent, a drug-eluting stent on a balloon, a drug-eluting stent on a drug-coated balloon, a stent on a drug-coated balloon, or a combination thereof. The medical device can be a balloon catheter.

In various aspects, the method further includes pleating and folding the coated balloon catheter.

In various aspects, the method further includes sterilizing the balloon catheter using electron beam radiation at 10 kGy to 100 kGy, or 15 kGy to 40 kGy, or less than or equal to 100 kGy and greater than or equal to 10 kGy and less than, equal to, or greater than 15 kGy, 20, 25, 30, 35, 40, 45, 50, 55, 60, 65, 70, 75, 80, 85, 90, or 95 kGy.

### Balloon Catheter.

Various aspects of the present disclosure provide a balloon catheter. The balloon catheter can include an elongated balloon. The balloon catheter can include a coating layer overlying an exterior surface of the balloon, wherein the coating layer includes the drug-releasing coating of the present disclosure.

The coating layer can have any suitable dose density of the therapeutic agent. The dose density can be measured with the balloon inflated at the nominal inflation pressure. For example, the coating layer can have a dose density of the therapeutic agent of 0.1 μg/mm² to 10 μg/mm², or 0.5 μg/mm² to 5 μg/mm², or less than or equal to 10 μg/mm² and greater than or equal to 0.1 μg/mm² and less than, equal to, or greater than 0.2 μg/mm², 0.3, 0.4, 0.5, 0.6, 0.7, 0.8, 0.9, 1, 1.2, 1.4, 1.6, 1.8, 2, 2.5, 3, 3.5, 4, 4.5, 5, 6, 7, 8, or 9 μg/mm².

The coating layer can have a zeta potential of −50 mV to 100 mV, or 15 mV to 100 mV, or 25 mV to 80 mV, or less than or equal to 100 mV and greater than or equal to −50 mV and less than, equal to, or greater than −45 mV, −40, −35, −30, −25, −20, −15, −10, −5, 0, 5, 10, 15, 20, 25, 30, 35, 40, 45, 50, 55, 60, 65, 70, 75, 80, 85, 90, or 95 mV.

The balloon catheter can have any suitable diameter. The balloon catheter can have a diameter (e.g., a diameter measured at nominal inflation pressure) of 0.5 mm to 20 mm, or 1 mm to 5 mm, or less than or equal to 20 mm and greater than or equal to 0.5 mm and less than, equal to, or greater than 1 mm, 1.5, 2, 2.5, 3, 3.5, 4, 4.5, 5, 6, 7, 8, 9, 10, 11, 12, 13, 14, 15, 16, 17, 18, or 19 mm. The balloon catheter can have any suitable length, such as a length of 5 mm to 200 mm, or 10 mm to 50 mm, or less than, equal to, or greater than 10 mm, 15, 20, 25, 30, 35, 40, 45, 50, 55, 60, 65, 70, 75, 80, 85, 90, 95, 100, 110, 120, 130, 140, 150, 160, 170, 180, or 190 mm.

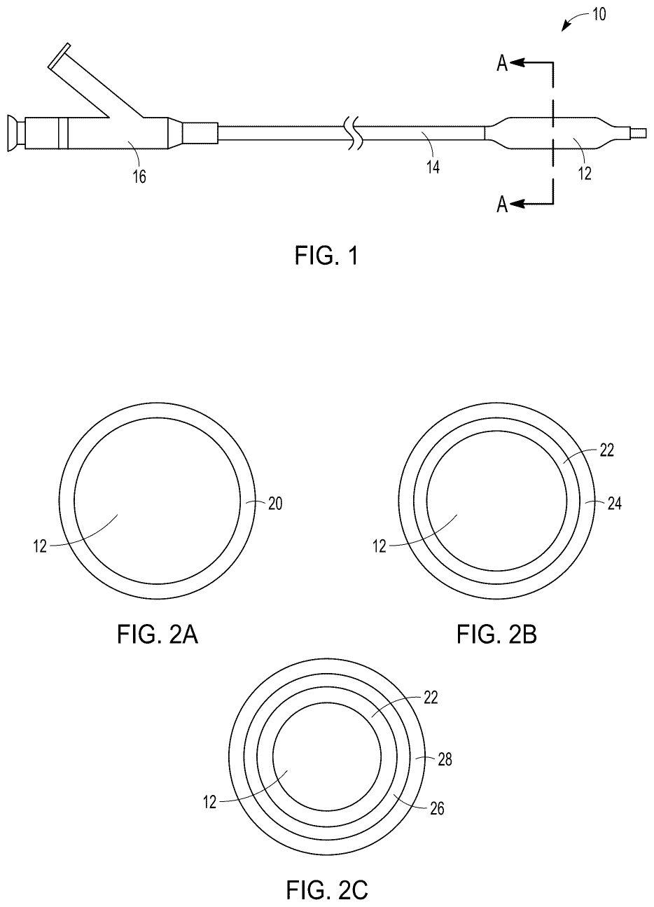

FIG. 1 illustrates a balloon catheter. The balloon catheter can be any suitable catheter for the desired use, including conventional cylindrical balloon catheters known to one of ordinary skill in the art. For example, balloon catheter 10 can include an expandable, inflatable balloon 12 at a distal end of the catheter 10, a handle assembly 16 at a proximal end of the catheter 10, and an elongate flexible member 14 extending between the proximal and distal ends. Handle assembly 16 can connect to and/or receive one or more suitable medical devices, such as a source of inflation media (e.g., air, saline, or contrast media). Flexible member 14 can be a tube made of suitable biocompatible material and having one or more lumens therein. At least one of the lumens is configured to receive inflation media and pass such media to balloon 12 for its expansion. The balloon catheter can be a rapid exchange or over-the-wire catheter and made of any suitable biocompatible material. The material of balloon 12 can include one or more of polyesters, polyamides, nylon 12, nylon 11, polyamide 12, block copolymers of polyether and polyamide, PEBAX®, polyurethanes, and block copolymers of polyether and polyester. The balloon catheters shafts can be constructed of polyether-amide block copolymers, polyamides, nylons, polyesters, polyethylene terephthalate, or any other semi-compliant to non-compliant polymer including their blends. The balloon catheter shaft can also be constructed using a rigid material such as stainless steel, polycarbonate, titanium, PEEK (polyether ether ketone), or any other rigid biocompatible material.

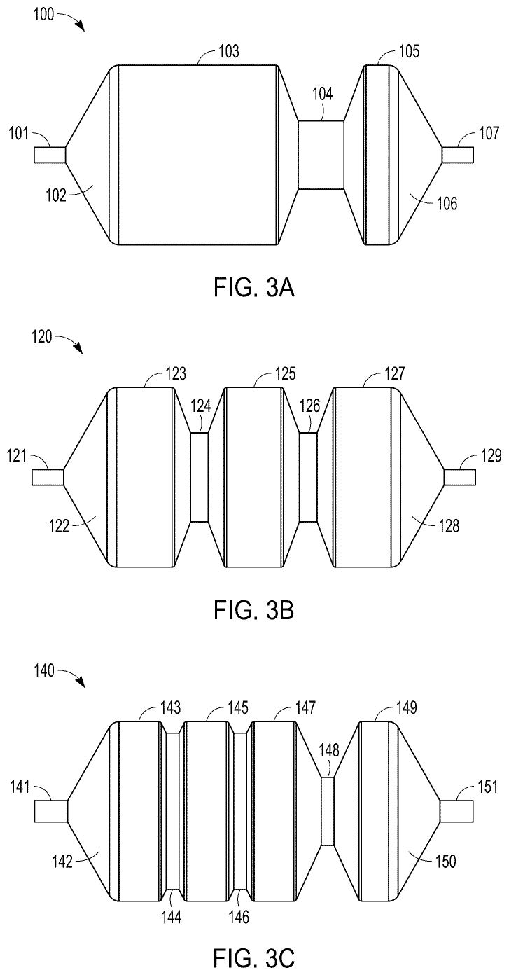

As illustrated in FIG. 2A, the balloon 12 is coated with a layer 20 that includes the drug-releasing coating of the present disclosure. In some embodiments, the device can optionally include an adherent layer. For example, as shown in the embodiment depicted in FIG. 2B, the balloon 12 is coated with an adherent layer 22. A layer 24 can be overlying the adherent layer, wherein the layer 24 includes drug-releasing coating of the present disclosure. The adherent layer, which is a separate layer underlying the drug coating layer, can improve the adherence of the drug coating layer to the exterior surface of the medical device and can protect coating integrity. For example, if drug and additive differ in their adherence to the medical device, the adherent layer can prevent differential loss of components and maintain drug-to-additive ratio in the coating during transit to a target site for therapeutic intervention. Furthermore, the adherent layer can function to facilitate rapid release of coating layer components off the device surface upon contact with tissues at the target site. In other embodiments, the device can include a topcoat layer. For example, as shown in the embodiment depicted in FIG. 2C, the balloon 12 is coated with an adherent layer 22, a coating layer 26 including therapeutic agent and overlying the adherent layer, and a topcoat layer 28. The topcoat layer can reduce loss of the drug layer before it is brought into contact with target tissues, for example during transit of the balloon 12 to the site of therapeutic intervention or during the first moments of inflation of balloon 12 before coating layer 20 is pressed into direct contact with target tissue.

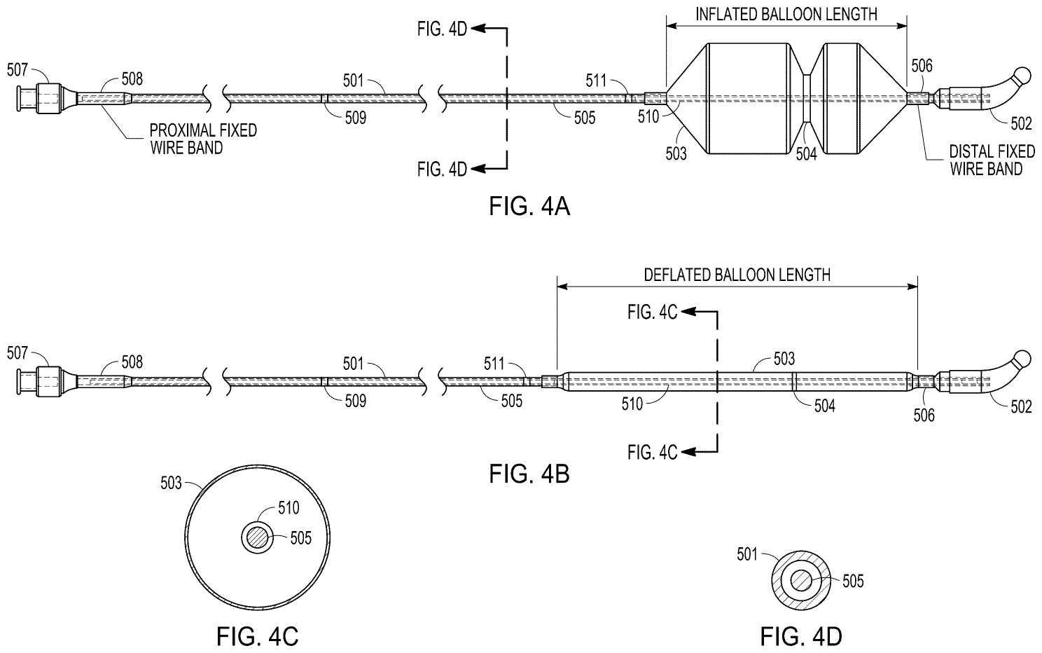

In some embodiments, the balloon can include one neck section and is free of other neck sections, such that the balloon includes two main sections separated by one neck section. The one neck section can have any suitable position on the balloon, such as approximately centered with respect to the balloon length, or off-center with respect to the balloon length. The one neck section can be off-center with respect to the length of the balloon and can be at a distal end of the balloon. An embodiment of the balloon including one neck section that is off-center with respect to the length of the balloon is illustrated in FIG. 3A.

In some embodiments, the balloon can include two neck sections and is free of other neck sections, such that the balloon includes three lobes separated by two neck sections. The two neck sections can have about the same diameter, or one of the neck sections can have a smaller diameter than the other neck sections. The two neck sections can be symmetrically or asymmetrically located with respect to the center of the balloon length. The three main sections can have approximately equal length or can have different lengths. FIG. 3B illustrates an embodiment of a balloon catheter having two neck sections with three main sections, wherein the neck sections are symmetrically located about the center of the length of the balloon, and wherein the three main sections of the balloon have about the same length. During use, the distal neck section (e.g., the neck section on the distal end of the balloon catheter which is inserted into the body first) can anchor and locate the balloon at the bladder neck, while the proximal neck section can be positioned in the prostatic urethra. In some embodiments, the distal main section of the balloon catheter can be free of the therapeutic agent.

In some embodiments, the balloon can include three necks and is free of other neck sections, such that the balloon includes four sections separated by the three necks. The three neck sections can be positioned in any suitable way along the length of the balloon. The four main sections formed by the three neck sections can have equal or different lengths. The three neck sections can have equal diameters, or different diameters. In some embodiments two of the neck sections have an equal diameter that is smaller than the diameter of the other neck section. FIG. 3C illustrates an embodiment of a balloon catheter having three neck sections with four main sections each having an approximately equal length, wherein two of the neck sections have an equal diameter that is smaller than the diameter of the other neck section.

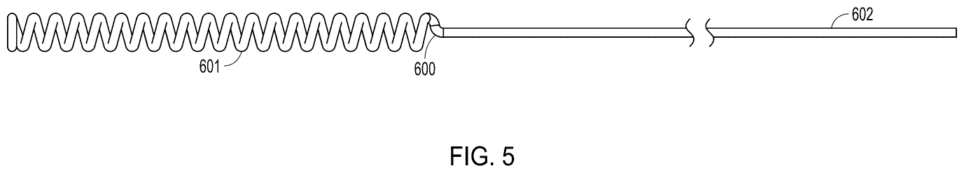

FIG. 4A to 4D illustrate a balloon catheter that includes an elongated rigid component or, core wire 505. FIG. 4A illustrates the embodiment with the balloon inflated and FIG. 4B illustrates the balloon in the non-expanded state. At the proximal end of the shaft, core wire 505 is attached to catheter shaft 501 under strain relief 508. Core wire 505 extends distally in catheter shaft 501. In some embodiments, catheter shaft is made from 72D PEBA polymer. The shaft 501 is made from a material that exhibits an amount of elasticity when under tension. Under balloon 503, core wire 505 is covered by a hypotube 510. Hypotube 510 provides lateral strength to core wire 505 so that it does not buckle when the balloon 503 is inflated. Near the distal end of the catheter, hypotube 510 and core wire 505 are bonded to tip 502. Tip extrusion 506 connects tip 502 to hypotube 510 and core wire 505. The space between shaft 501 and core wire 505 is the inflation lumen for balloon 503, with the interior of balloon 503 being in fluid communication with Luer hub 507. While this embodiment can be used with any suitable balloons of the invention, FIGS. 4A and 4B show balloon 510 503 with one neck, with polyethylene fiber 504 used to reinforce the neck.

The elongated rigid component can have a cross-sectional profile that is cylindrical, tapered, rectangular, hexagonal, or another shape and can be made from metal or a non-metallic material that is relatively non-compressible. The elongated component can run from the proximal side of the balloon to the distal side of the balloon, or from a location proximal to the proximal side of the balloon to the distal side of the balloon. The elongated component can float freely within a central lumen of the catheter shaft, can be positioned in a dedicated lumen in a multilumen catheter shaft, or can run longitudinally on the outside of the main catheter shaft. The elongated component can be anchored at a single point, at two points, or at more than two points along the catheter shaft. The elongated component can be anchored by thermally fusing it directly to the catheter shaft, adhesively or chemically bonding it to the catheter shaft, swaging or crimping to one or more portions of the catheter, overmolding, or via any other suitable method. The elongated component can be reinforced along its entire length or along certain sections such as under the balloon to prevent buckling; for example, the elongated metallic component can be a reinforced wire. The reinforcement can be constructed using any rigid material such as stainless steel, Nitinol (i.e., nickel titanium alloy), steel, tungsten, iridium, superalloys contain elements, including nickel (Ni) chromium (Cr), aluminum (Al), titanium (Ti), tungsten (W), niobium (Nb), tantalum (Ta) and cobalt (Co), or Polyether ether ketone (PEEK) and can have any suitable cross-sectional shape. In some embodiments, the reinforcement is a tube that is cylindrical, rectangular, hexagonal, or having any suitable outer profile. The elongated component can be disposed inside of the reinforcement tube as shown in FIG. 4C, or along the outside of the reinforcement tube.

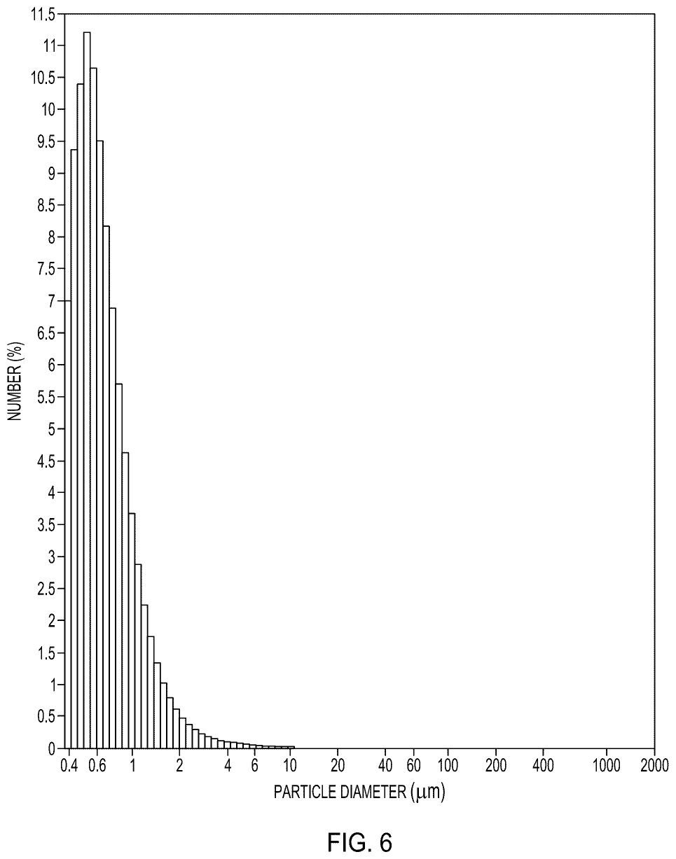

As shown in FIGS. 4A and 4B, the catheter can include a balloon length-control mechanism which stretches and elongates the balloon when it is in a deflated state, giving the balloon a smaller cross-sectional deflated profile for tracking through the body lumen and for removal after treatment. When the balloon is inflated, the length-control mechanism can allow the balloon to shorten in overall length and inflate to the predetermined inflated diameter and length for the balloon (e.g., as created during the molding or forming process). In one embodiment the force generated from balloon inflation could be transferred from the distal end of the balloon, such as via a balloon bond to the elongated metallic component or via a connection between the catheter tip and the elongated metallic component, back through the catheter shaft by the elongated rigid metallic component to the catheter shaft proximal to the balloon, such as via a connection between the elongated metallic component and the catheter shaft at the proximal end of the balloon or proximal to the proximal end of the balloon. This transfer of force to the catheter shaft would allow the catheter shaft material to act as a spring while working in the elastic region of the catheter shaft material's stress-strain curve. Energy can be stored in the catheter shaft material during balloon inflation, when the catheter shaft is elongated under tension due to balloon inflation and can be released by the catheter shaft to press on elongated metallic component during deflation to elongate the balloon. In some embodiments, a spring oriented longitudinally along the catheter shaft can be used to store and release force for the balloon length-control mechanism. FIG. 5 illustrates an embodiment of the spring, 600, which can be used as an alternative to core wire 505 shown in FIGS. 4A and 4D. Referring to FIG. 5, spring 600 has a spring section 601 and wire section 602. In some embodiments, spring section 601 can be located at the proximal end of the catheter shaft. The spring can be located within a lumen in the catheter shaft, outside the lumen but within the catheter shaft, or outside the catheter shaft. The spring can be within the balloon, or can be located separately from the balloon, such as proximally to the proximal end of the balloon, or a combination thereof. As compared to the length of the inflated balloon, the elongated length of the deflated balloon can be about 0.1 mm longer to about 100 mm longer, or less than, equal to, or greater than about 0.1 mm longer, 0.2, 0.4, 0.6, 0.8, 1, 1.5, 1, 2.5, 3, 4, 5, 6, 8, 10, 12, 15, 20, 25, 30, 40, 50, 60, 70, 80, 90, or about 100 mm longer or more. The catheter shaft can include various materials to achieve the desired amount of force to elongate the balloon, such as polyamides, nylon (e.g., nylon 6,6, or nylon 12), a polyether block amide (PEBA) (e.g., 35D PEBA, 55D PEBA, or 72D PEBA), polyurethanes, silicones, rubbers, another thermoplastic polymer, or a combination thereof. The catheter shaft can be uniform in composition or can include a combination of materials that are distributed along one or more portions of the catheter shaft to create the desired elongation force. Different materials can yield different elastic strain and different force applied to the elongated rigid metallic component for balloon elongation. The catheter shaft can be an extruded catheter shaft.

### Method for Treating a Target Location.

Various aspects of the present disclosure provide a method for treating a target location. The method can include inserting a medical device including the drug-releasing coating of the present disclosure thereon into the target location. The method can include allowing the drug-releasing coating to release the therapeutic agent onto the tissue at the target location. The method can also include withdrawing the medical device from the target location. In various aspects, the medical device is a balloon catheter, a drug-coated catheter, a drug-eluting stent, a drug-eluting stent on a balloon, a drug-eluting stent on a drug-coated balloon, a stent on a drug-coated balloon, or a combination thereof. In various aspects, the medical device is a balloon catheter.

The target location can be any suitable target location. The target location can include a portion of the urinary tract, such as the urethra (e.g., for treatment of urethral strictures or prostatic urethral strictures), the prostate (such as for treatment of benign prostatic hyperplasia strictures or prostate cancer), the ureter (such as for treatment of ureteral strictures), the bladder neck (such as for treatment of bladder neck strictures or stenoses), or the bladder.

The target location can include a portion of the gastrointestinal (GI) tract or digestive system, such as the esophagus (e.g., for treatment of esophageal strictures, achalasia strictures, esophageal strictures of eosinophilic esophagitis, or Barrett's esophagus), the stomach (such as for treatment of stomach strictures or gastrectomy-induced stomach strictures), the small intestine (such as for treatment of small intestine strictures, duodenum strictures, jejunum strictures, or ileum strictures), or the colon (such as for treatment of colon strictures, colorectal strictures, large intestine strictures, rectum strictures, ileoanal J-pouch strictures, ileocolonic strictures, or general gastrointestinal strictures). The target location can also include a portion of the biliary tract (such as for treatment of biliary strictures or bile duct strictures) or anastomosis sites, such as surgical anastomosis-induced strictures following procedures like bowel resection, gastric bypass, or J-pouch formation.

The target location can include a portion of the vascular system, such as arteries (e.g., for treatment of coronary artery stenosis, carotid artery stenosis, brachial artery stenosis, radial artery stenosis, renal artery stenosis, iliac artery stenosis, superficial femoral artery stenosis, popliteal artery stenosis, anterior tibial artery stenosis, posterior tibial artery stenosis, peroneal artery stenosis, or other arteries of the foot), veins, or arteriovenous fistulas (AVF) or arteriovenous grafts (AVG), such as stenosed AVFs and AVGs. The target location can also include in-stent restenosis at any vascular location and stenosed heart valves, such as aortic or mitral valves.

The target location can include a nonvascular lumen, such as the sinus (including sinus strictures of the paranasal sinuses) or the vagina (including vaginal strictures or stenosis).

The target location can include any body lumen that includes a stricture or stenosis, such as nonvascular lumens (that are normally without blood) or vascular lumens (that are normally with blood). This can includes site of prior surgical intervention or cancer treatment, such as radiation-induced strictures or strictures resulting from endoscopic mucosal resection (EMR) or endoscopic submucosal dissection (ESD), or sites of chronic inflammatory disease, such as Crohn's disease, ulcerative colitis, or other inflammatory bowel disease (IBD)-induced strictures. The target location can include anastomotic strictures between any two body structures, such as those occurring after surgery.

### Method for Treating a Vascular Condition.

Various aspects of the present disclosure provide a method for treating a vascular condition. The method includes providing the balloon catheter of the present disclosure that includes the drug-releasing coating of the present disclosure thereon. The method can include inserting the balloon catheter into a target site in a vascular lumen. The method can include inflating the balloon catheter to contact the coating layer with tissue at the target site. The method can also include deflating and removing the balloon catheter.

In various aspects, the tissue at the target site can retain 1% to 100% of the therapeutic agent in the coating layer after the balloon catheter has been removed from the target site, or 20% to 90% of the therapeutic agent in the coating layer, or 50% to 90% of the therapeutic agent in the coating layer, or less than or equal to 100% and greater than or equal to 1% and less than, equal to, or greater than 2%, 3, 4, 5, 6, 8, 10, 12, 14, 16, 18, 20, 25, 30, 35, 40, 45, 50, 55, 60, 65, 70, 75, 80, 85, 90, 92, 94, 95, 96, 97, 98, or 99%.

### Method of Making Polymer-Encapsulated Drug Particles.

Various aspects of the present disclosure provide a method of making the polymer-encapsulated drug particles that are included in the drug-releasing coating of the present disclosure. The method can include dissolving a therapeutic agent (e.g., sirolimus), a polymer (e.g., PLGA), and a first ionic or zwitterionic additive in a solvent (e.g., dichloromethane) to form an oil phase. The method can include dispersing the oil phase in a water phase containing polyvinyl alcohol using high-pressure microfluidic apparatus. The method can include mixing the oil-water mixture with excess water for stabilization and solvent evaporation. The method can include centrifuging to separate microspheres. The method can include lyophilizing to obtain dried microsphere powder.

The oil phase can include any suitable solvent. The oil phase can include dichloromethane.

The high-pressure microfluidic apparatus can operate at any suitable pressure, such as at a pressure of 20,000 psi to 40,000 psi, or less than or equal to 40,000 psi and greater than or equal to 20,000 psi and less than, equal to, or greater than 22,000 psi, 24,000, 26,000, 28,000, 30,000, 32,000, 34,000, 36,000, or 38,000 psi.

The polymer-encapsulated drug particles (e.g., microspheres) can have any suitable mean largest dimension or mean diameter (D50), such as 0.1 μm to 10 μm, 0.5 μm to 5 μm, or 1 μm to 5 μm, or less than or equal to 10 μm and greater than or equal to 0.1 μm and greater than, equal to, or less than 0.2 μm, 0.3, 0.4, 0.5, 0.6, 0.7, 0.8, 0.9, 1, 1.2, 1.4, 1.6, 1.8, 2, 2.2, 2.4, 2.6, 2.8, 3, 3.5, 4, 4.5, 5, 5.5, 6, 6.5, 7, 7.5, 8, 8.5, 9, or 9.5 μm. The method can further include using a narrow size distribution of the microspheres.

### EXAMPLES

Various aspects of the present invention can be better understood by reference to the following Examples which are offered by way of illustration. The present invention is not limited to the Examples given herein.

Throughout the Examples, unless otherwise indicated, the stretch ratio was calculated as the ratio of the diameter of the nominal balloon to the diameter of the body lumen stricture at the location of treatment. The diameter of the body lumen at the location of treatment is the normal diameter for the body lumen at the location of treatment and can be calculated as the average of the diameters of healthy tissue adjacent to the stricture, stenosis, or lesion, that is proximal and distal of the stricture or stenosis or lesion of the lumen. The inflated balloon diameter was about equal to the nominal balloon diameter for the pressure used during the inflation period and was within 10% of the nominal balloon diameter.

### Part I. Preclinical and Bench Testing.

### Example I-1. Preparation of Coating Solutions

Formulation 23: 50-150 mg (0.06-0.18 mmole) paclitaxel, 5-75 mg pentaerythritol ethoxylate (15/4), 10-200 mg pentaerythritol ethoxylate (3/4), and 1-6 ml ethanol were mixed.

Formulation 24: 50-150 mg (0.06-0.2 mmole) paclitaxel, 25-300 mg trimethylpropane ethoxylate (Mw-170)), and 1-6 ml ethanol were mixed.

Formulation S16: 45-200 mg (0.05-0.22 mmole) sirolimus, 5-75 mg pentaerythritol ethoxylate (15/4), 23-100 mg Brij 52 Cetyl Ether, and 1-6 ml (10/90 v/v) methanol/water were mixed.

Formulation S21: 45-200 mg (0.05-0.22 mmole) sirolimus, 5-75 mg monolaurin, and 1-6 ml (10/90 v/v) methanol/water were mixed.

Formulation S22: 45-300 mg (0.05-0.22 mmole) sirolimus, 5-80 mg pentaerythritol ethoxylate (15/4), 23-100 mg Brij 52 Cetyl Ether, 23-150 mg D-α-Tocopherol polyethylene glycol 1000 succinate (TPGS), and 1-6 ml (25/75 v/v) methanol/water were mixed.

Formulation S44: 45-300 mg (0.05-0.22 mmole) sirolimus, 5-80 mg pentaerythritol ethoxylate (15/4), 23-100 mg dodecyl glycerol, and 1-6 ml (25/75 v/v) methanol/water were mixed.

Formulation S47a: 45-200 mg (0.05-0.22 mmole) sirolimus, 4.5-20 mg polyoxyethanyl α-tocopheryl sebacate, 23-100 mg dodecyl glycerol, and 1-6 ml (25/75 v/v) methanol/water were mixed.

Formulation S47b: 45-200 mg (0.05-0.22 mmole) sirolimus, 4.5-20 mg polyoxyethanyl α-tocopheryl sebacate, 23-100 mg dodecyl glycerol, and 1-6 ml (25/75 v/v) ethanol/water were mixed.

Formulation S57: 45-200 mg (0.05-0.22 mmole) sirolimus, 4.5-20 mg polyoxyethanyl α-tocopheryl sebacate, 23-100 mg cholesteryl acetate, 23-100 mg dodecyl glycerol, and 1-6 ml (25/75 v/v) ethanol/water were mixed.

Formulation S58: 45-200 mg (0.05-0.22 mmole) sirolimus, 4.5-20 mg polyoxyethanyl α-tocopheryl sebacate, 23-100 mg mPEG-cholesterol, MW 5 k, 23-100 mg dodecyl glycerol, and 1-6 ml (25/75 v/v) ethanol/water were mixed.

Formulation S59: 45-200 mg (0.05-0.22 mmole) sirolimus, 23-100 mg mPEG-cholesteryl MW 5 k, 23-100 mg dodecyl glycerol, and 1-6 ml (25/75 v/v) ethanol/water were mixed.

Formulation CC6F2: 45-200 mg (0.05-0.22 mmole) sirolimus, 4.5-20 mg polyoxyethanyl α-tocopheryl sebacate, 23-100 mg cholesteryl decylate, 23-100 mg dodecyl glycerol, and 1-6 ml (20/80 v/v) cyclohexane/water were mixed.

Formulation CC6F4: 45-200 mg (0.05-0.22 mmole) sirolimus, 4.5-20 mg polyoxyethanyl α-tocopheryl sebacate, 23-100 mg cholesteryl acetate, 23-100 mg dodecyl glycerol, and 1-6 ml (20/80 v/v) cyclohexane/water were mixed.

Formulation S16 was prepared by using a rotor-stator process. 123 mg of sirolimus was added to a vial along with 3 mL of water. Next the rotor-stator was used to conduct a particle size reduction on the sirolimus. Next a premix was made in a separate vial consisting of; 55 mg of Brij 52 cetyl ether, 27.5 mg of pentaerythritol ethoxylate 15/4, 0.36 mL of methanol and 0.64 mL of water. This premix was added to the vial with the drug and mixed with the rotor-stator for approximately 5 minutes. This solution was used to coat balloons.

Formulation S22 was prepared by using a low energy bead milling process. A jar mill with 5 mm diameter by 5 mm length yttrium stabilized zirconium grinding beads was used to conduct a sirolimus particle size reduction. 200 mg of sirolimus was added to a jar along with 8 mL of grinding beads. Next a premix was made in a separate vial consisting of; 100 mg of TPGS, 0.5 mL of methanol and 3 mL of water. This premix was added to the jar with the drug and milled for approximately 3-hours. Lastly a second premix of 25 mg of Brij 52 cetyl ether, 75 mg of pentaerythritol ethoxylate 15/4, 0.75 mL of methanol and 0.75 mL of water was prepared. All materials were fully solubilized in the second premix. The second premix was added to the jar containing the drug and milled overnight for approximately 16 hours. This solution was used to coat balloons.

Formulation S59 was prepared by using a high energy ultrasonic probe process. An ultrasound system, Sonics Vibra-cell VCX 130 with 6 mm probe, was used to conduct a sirolimus particle size reduction. 200 mg of sirolimus was added to a vial. Then 3 mL of ethanol/water (25/75 v/v) was added to separate vial. The vial was placed in an ice water bath and the mixture was sonicated with the ultrasound system for 5 minutes. Next a premix was made by dissolving 200 mg of dodecyl glycerol and 200 mg of mPEG-cholesteryl MW 5 k into 0.72 mL of ethanol. Then 0.24 mL of water was added to the second premix. All materials were fully solubilized in the second premix. The second premix was added to the drug suspension vial and sonicated further for 5 minutes and used to coat balloons.

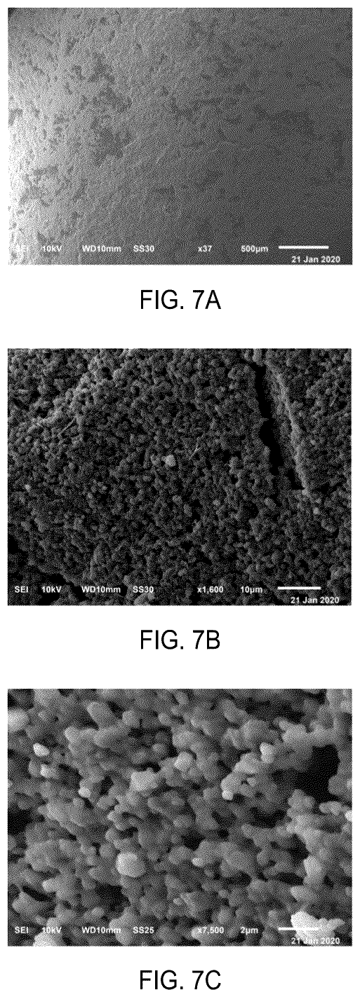

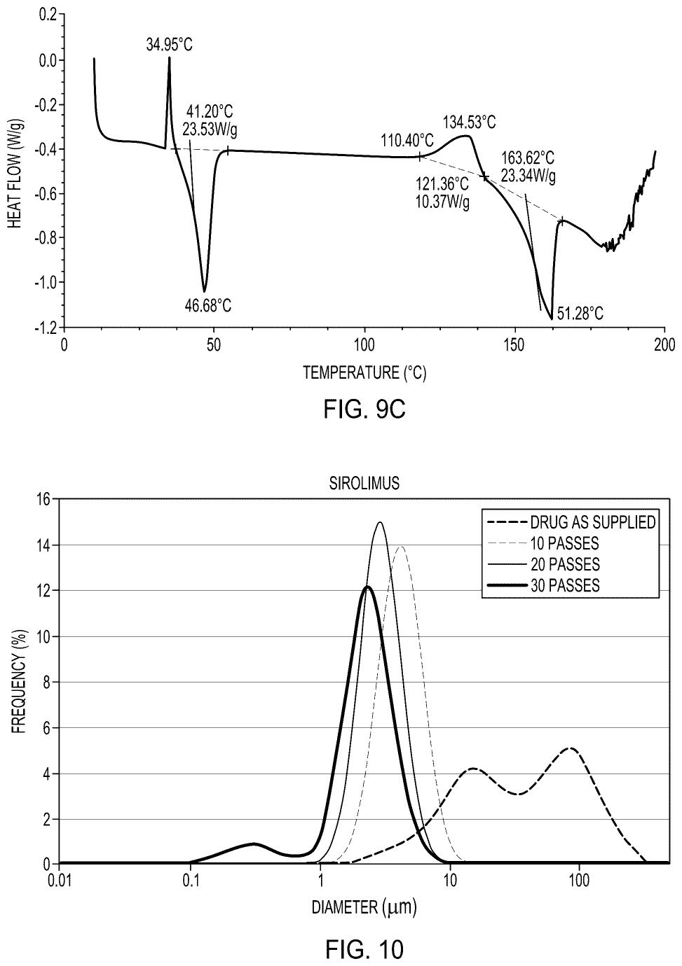

Formulation S47a was prepared by using a high-pressure homogenization process. A Microfluidizer was used to conduct a sirolimus particle size reduction. The Microfluidizer has a product recirculation loop and a heat exchanger to allow product cooling. 1.4 g of polyoxyethanyl α-tocopheryl sebacate was premixed with 34.5 mL of water. The premix was added to the microfluidizer. Once the premix was charged into the homogenizer vessel the microfluidizer was put into recirculation mode at a pressure of 30,000 psi. 4 g of sirolimus was slowly added to the vessel. Then the aqueous drug suspension was recirculated for 2 hours while periodically checking the particle size via laser light diffraction. The particle size was reduced from a D₉₀ of 124 μm to a D₉₀ of 3.78 μm. See FIG. 10, which illustrates a diagram of the sirolimus particle size reduction obtained with a high-pressure homogenizer. Next a second premix was made by dissolving 2 g of dodecyl glycerol and 1.29 g of polyoxyethanyl α-tocopheryl sebacate in 9 mL of ethanol. Then 3 mL of water was added to the second premix. All materials were fully solubilized in the second premix. The second premix was added to the drug suspension already being recirculated in the microfluidizer. The final mixture was further recirculated for an additional 1 hour. Coating solution S47a were characterized for particle size using laser light diffraction. Results are shown in FIG. 6. The D₉₀ was 1.149 μm, D₇₅ was 0.817 μm, D₅₀ was 0.607 μm, D₂₅ was 0.489 μm and the Dio was 0.425 μm. Calculations were from 0.375 μm to 2000 μm. Mean was 0.746 μm; median was 0.607 μm; mean/median ratio was 1.230; mode was 0.520 μm; S.D. was 0.532 μm; variance was 0.283 μm²; C.V. was 71.4%; skewness was 6.796 right skewed; kurtosis was 80.59 leptokurtic.

Formulation S47a was coated on 12 mm diameter by 50 mm length balloons using the following process. A special coating machine was used to dispense Formulation S47a onto the surface of the balloon. The solution was pumped to a dispense nozzle near the rotating balloon catheter. The balloon catheter was mounted into a fixture such that the balloon longitudinal axis was horizontal and the balloon could be spun on its longitudinal axis at a precise speed. The dispense nozzle was mounted onto a linear stage to allow for controlled motion of the nozzle along the length of the balloon while simultaneously dispensing liquid Formulation S47a onto the balloon. The nozzle made seven back and forth passes along the length of the balloon during coating solution dispensing and the balloon was maintained at a rotational speed of 75 RPM. After dispensing the nozzle was moved away and the balloon maintained rotation while simultaneously using a hot air gun to further dry the coating. The hot air nozzle was directed at the balloon and the temperature of the hot air was maintained at 120° F. until the balloon was completely dried for 3 minutes. This process was repeated for all balloons to be coated. Once the balloon were dried they were put in a humidification oven for 4 hours at 45° C. and 80% RH. After that the balloons were pleat and folded and sheathed. Then they were placed in Tyvek packaging and sent out to get ethylene oxide sterilized.

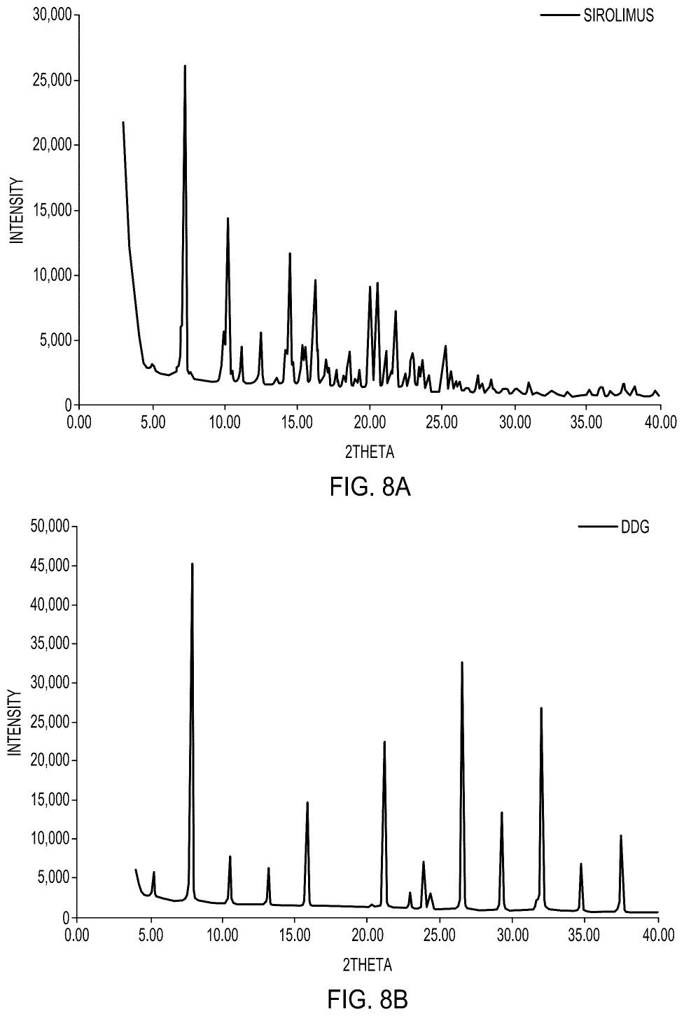

Sterilized balloons with S47a coating on them where characterized using scanning electron microscopy the results show that there were no observable particles greater than 10 μm and many of the particles were sub 1 micron in size. See FIG. 7, which illustrates SEM images of the sirolimus-coated balloon, with image (A) showing 37×, image (B) showing 1,600×, image (C) showing 7,500×, in accordance with various aspects.

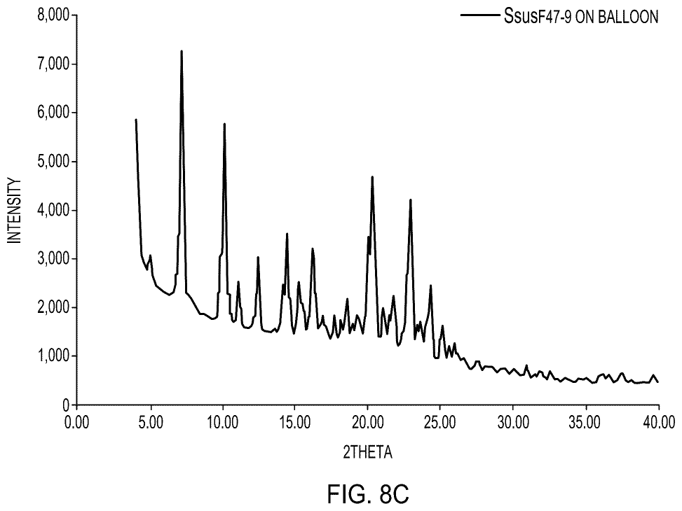

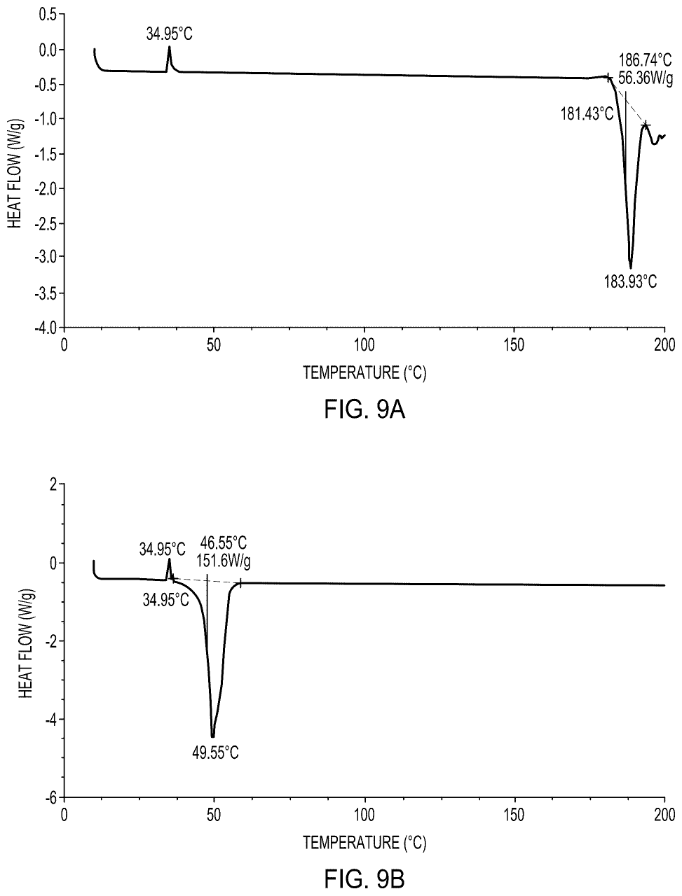

Sterilized balloons with S47a coating on them along with pure sirolimus samples and pure additive (DDG-dodecyl glycerol) samples where characterized using powder x-ray diffraction and modulated Differential Scanning Calorimetry. The results showed that the sirolimus in the sterilized coating S47a is crystalline. See FIG. 8. The DSC measurements were used to calculate the percent crystallinity of the coating. The results show the Sirolimus was 77.3 to 93.9% crystalline by weight. The DSC scans also showed that the melting temperature of the additive (DDG-dodecyl glycerol) was reduced 2-4° C. and the melting temperature for the drug was reduced 25-27° C. See FIGS. 9A-C and Table 4. FIGS. 9A-C illustrate diagrams of DSC scans of (A) crystalline sirolimus, (B) dodecyl glycerol, (C) drug (S47A)-coated balloon.

TABLE 4
DSC data of coating on balloon from Formulation S47a.

| | 1^{st} | 1^{st} | 1^{st} | 2^{nd} | 2^{nd} | 2^{nd} | 3^{rd} | 3^{rd} | |
|---|---|---|---|---|---|---|---|---|---|
| | onset | peak | Peak | onset | peak | Peak | peak | Peak | % |
| | temp. | temp. | enthalpy | temp. | temp. | enthalpy | temp. | enthalpy | crystallinity |
| Specimen | (° C.) | (° C.) | (J/g) | (° C.) | (° C.) | (J/g) | (° C.) | (J/g) | sirolimus |
| Sample 1 | 36.48 | 46.56 | 17.04 | 124.96 | 140.76 | 10.47 | 163.4 | 25.92 | 83.0 |
| Sample 2 | 36.9 | 46.85 | 16.18 | 124.96 | 139.79 | 8.07 | 163.14 | 24.15 | 77.3 |
| Sample 3 | 36.69 | 46.75 | 20.58 | 124.75 | 139.16 | 10.14 | 164.21 | 29.08 | 93.1 |
| Sample 4 | 37.12 | 46.97 | 18.91 | 124.54 | 138.97 | 8.55 | 162.7 | 26.58 | 85.1 |
| Sample 5 | 36.9 | 46.73 | 20.58 | 124.96 | 138.73 | 8.83 | 162.69 | 29.32 | 93.9 |
| Average | 36.82 | 46.77 | 18.66 | 124.83 | 139.48 | 9.21 | 163.23 | 27.01 | 86.5 |
| S.D. | 0.24 | 0.15 | 2.01 | 0.19 | 0.82 | 1.04 | 0.63 | 2.19 | 7.0 |

### Example I-2. Studies on Various Coating Formulations

Porcine Animal Study with Formulation S79.

Formulation S79, an aqueous microcrystalline sirolimus suspension with dissolved 1,2-dilauroyl-sn-glycero-3-phosphatidylcholine, was prepared by using a high-pressure homogenization process. A Microfluidizer was used to conduct a sirolimus crystal particle size reduction. The Microfluidizer has a product recirculation loop and a tube in shell heat exchanger to allow for product cooling. The cooling media that was recirculated through the heat exchanger was maintained at 5° C. using a Recirculating Chiller. A premix of 4.0 g of 1,2-dilauroyl-sn-phosphatidylcholine dissolved in 9.4 mL of ethanol was added to 28.1 mL of water. This premix was added to the microfluidizer. Once the premix was charged into the homogenizer vessel the microfluidizer was put into recirculation mode at a pressure of 30,000 psi. 4.0 g of sirolimus was slowly added to the vessel. Then the aqueous drug suspension was recirculated for 2 hours. Lastly the homogenized mixture was packed out into a trace clean bottle and 12.5 mL of 75/25 water/ethanol was chased through the microfluidizer and added to the bottle to recover as much crystalline drug particles as possible. Formulation S79 was uniformly coated onto balloons of various sizes for acute drug transfer testing in a pig. The balloons were coated using an automated coating machine that precisely dispensed a prespecified volume of coating solution onto the surface of the balloon. The prespecified volume of solution was calculated based on the coating solution concentration and the desired nominal drug dose per balloon. The balloon sizes, diameter×length, were 6×30 and 18×65. The amount of drug per square millimeter for each balloon was 2. This gave nominal drug dosing of 1165 μg and 7572 μg for each respective balloon size. The 6 mm balloons were used in the pig's urethra with a stretch ratio of 1.0 to 2.0. Lastly the 18 mm balloons were used in the pig's esophagus, small intestine, and colon with stretch ratios of 1.0-2.0. For the urological and gastrointestinal treatments the balloon was tracked into position and the coating was allowed to hydrate for 1 minute prior to inflation. For the gastrointestinal treatments a gastroscope, enteroscope, or colonoscope was used to visualize the treatment site and flush the wall of the treatment site prior to use of the DCB. After all treatments, the pig was alive for 24 hours prior to sacrifice, then the treated tissue was excised in the necropsy lab and assayed for drug content. The measured sirolimus drug concentration in the various tissues can be seen in Table 5. The residual amount of drug on the balloon after treatment was measured and can be seen in Table 6.

TABLE 5
Formula S79 measured drug concentrations.

| Treatment site | Sirolimus Concentration (ug/g) |
|---|---|
| Esophagus - Treatment (Prox) | 0.255 |
| Esophagus - Treatment (Dist) | 0.224 |
| Duodenum - Treatment (Dist) | 0.680 |
| Colon - Treatment (Mid) | 5.27 |

TABLE 6
Formula S79 residual drug on balloon after treatment.

| Treatment Location | Residual Balloon Content (% of Dose) |
|---|---|
| Colon - Treatment (Mid) | 52.40% |
| Duodenum - Treatment (Dist) | 2.50% |
| Esophagus - Treatment (Prox) | 15.90% |
| Esophagus - Treatment (Dist) | 23.00% |

Porcine Animal Study with Formulation S96, S97, and S98.

Formula S96 Sirolimus Coating Solution Preparation. Formulation S96, an aqueous microcrystalline sirolimus suspension encapsulated in lipids with dissolved 1,2 dihexanoyl-sn-glycero-3-phosphatidylcholine, 1,2-dilauroyl-sn-glycero-3-phosphatidylcholine and 1,2-dipalmitoyl-sn-glycero-3-ethylphosphocholine (chloride salt), was prepared by using a sonic probe process. A first premix of 75 mg cetyl palmitate, 150 mg of cholesterol stearate and 75 mg of cholesterol acetate was dissolved in 0.7 mL of cyclohexane. Next 200 mg of crystalline sirolimus was added to this first premix. An ultrasound system, Sonics Vibra-cell VCX 130 with 6 mm probe, was used to conduct a sirolimus particle size reduction on the first premix. A second aqueous premix was made by adding 0.35 mL of ethanol to 5 mL of water. While maintaining ultrasonic agitation the first premix was added to the second premix to make a solid particle in oil with water emulsion. The emulsion was mixed with sonic agitation for 5 minutes to allow the lipids to solidify thus coating the sirolimus drug particles. Lastly a third premix was made by dissolving 27.78 mg of 1,2 dihexanoyl-sn-glycero-3-phosphatidylcholine, 138.89 mg of 1,2-dipalmitoyl-sn-glycero-3-ethylphosphocholine (chloride salt), and 166.67 mg of 1,2-dilauroyl-sn-glycero-3-phosphatidylcholine in 1.635 mL of ethanol. Then 1.015 mL of water was added to the third premix. The third premix was added to the drug particle emulsion mixture to complete the coating solution.

Formula S97 Sirolimus Coating Solution Preparation. Formulation S97, an aqueous microcrystalline sirolimus suspension encapsulated in lipids with dissolved 1,2 dihexanoyl-sn-glycero-3-phosphatidylcholine, 1,2-dilauroyl-sn-glycero-3-phosphatidylcholine, Poly(1-vinylpyrrolidone-co-2-dimethylaminoethyl methacrylate), and DC-Cholesterol, was prepared by using a sonic probe process. A first premix of 75 mg cetyl palmitate, 150 mg of cholesterol stearate and 75 mg of cholesterol acetate was dissolved in 0.7 mL of cyclohexane. Next 200 mg of crystalline sirolimus was added to this first premix. An ultrasound system, Sonics Vibra-cell VCX 130 with 6 mm probe, was used to conduct a sirolimus particle size reduction on the first premix. A second aqueous premix was made by adding 0.35 mL of ethanol to 5 mL of water. While maintaining ultrasonic agitation the first premix was added to the second premix to make a solid particle in oil with water emulsion. The emulsion was mixed with sonic agitation for 5 minutes to allow the lipids to solidify thus coating the sirolimus drug particles. Lastly a third premix was made by dissolving 50.0 mg of 1,2 dihexanoyl-sn-glycero-3-phosphatidylcholine, 50.0 mg of Poly(1-vinylpyrrolidone-co-2-dimethylaminoethyl methacrylate), 66.7 mg of DC-cholesterol and 166.67 mg of 1,2-dilauroyl-sn-glycero-3-phosphatidylcholine in 1.635 mL of ethanol. Then 1.015 mL of water was added to the third premix. The third premix was added to the drug particle emulsion mixture to complete the coating solution.

Formula S98 Sirolimus Coating Solution Preparation. Formulation S98, an aqueous microcrystalline sirolimus suspension encapsulated in lipids with dissolved 1,2 dihexanoyl-sn-glycero-3-phosphatidylcholine, 1,2-dilauroyl-sn-glycero-3-phosphatidylcholine and DC-Cholesterol, was prepared by using a sonic probe process. A first premix of 75 mg cetyl palmitate, 150 mg of cholesterol stearate and 75 mg of cholesterol acetate was dissolved in 0.7 mL of cyclohexane. Next 200 mg of crystalline sirolimus was added to this first premix. An ultrasound system, Sonics Vibra-cell VCX 130 with 6 mm probe, was used to conduct a sirolimus particle size reduction on the first premix. A second aqueous premix was made by adding 0.35 mL of ethanol to 5 mL of water. While maintaining ultrasonic agitation the first premix was added to the second premix to make a solid particle in oil with water emulsion. The emulsion was mixed with sonic agitation for 5 minutes to allow the lipids to solidify thus coating the sirolimus drug particles. Lastly a third premix was made by dissolving 25.0 mg of 1,2 dihexanoyl-sn-glycero-3-phosphatidylcholine, 108.3 mg of DC-cholesterol and 200.0 mg of 1,2, dilauroyl-sn-glycero-3-phosphatidylcholine in 1.635 mL of ethanol. Then 1.015 mL of water was added to the third premix. The third premix was added to the drug particle emulsion mixture to complete the coating solution.

Formulations S96, S97, and S98 were uniformly coated onto balloons of various sizes for acute drug transfer testing in a pig. The balloons were coated using an automated coating machine that precisely dispensed a prespecified volume of coating solution onto the surface of the balloon. The prespecified volume of solution was calculated based on the coating solution concentration and the desired nominal drug dose per balloon. The balloon sizes, diameter×length, were 6×30 and 18×65. The amount of drug per square millimeter for each balloon was 2 micrograms/mm² at nominal inflation pressure. This gave nominal drug dosing of 1165 μg and 7572 μg for each respective balloon size The 6 mm balloons were used in the pig's urethra with a stretch ratio of 1.0 to 2.0. Lastly the 18 mm balloons were used in the pig's esophagus, small intestine, and colon with stretch ratios of 1.0-2.0. For the urological and gastrointestinal treatments the balloon was tracked into position and the coating was allowed to hydrate for 1 minute prior to inflation. For the gastrointestinal treatments a gastroscope, enteroscope, or colonoscope was used to visualize the treatment site and flush the wall of the treatment site prior to use of the DCB. After all treatments, the pig was alive for 24 hours prior to sacrifice, then the treated tissue was excised in the necropsy lab and assayed for drug content. The measured sirolimus drug concentration in the various tissues can be seen in Table 7.

TABLE 7
Formulations S96, S97, and S98 measured
drug concentrations in various tissues.

| Sample | Formula | Sirolimus [ug/g] |
|---|---|---|
| Esophagus - prox | S98 | 0.00 |
| Esophagus - mid | S96 | 0.00 |
| Esophagus - dist | S97 | 0.00 |
| Duodenum - prox | S97 | 0.12 |
| Duodenum - dist | S96 | 0.00 |
| Colon - prox | S96 | 2.65 |
| Colon - dist | S98 | 1.56 |
| Urethra - prox | S96 | 0.17 |
| Urethra - mid | S97 | 0.11 |
| Urethra - dist | S98 | 0.15 |

### Polymer Encapsulated Drug Particles (PEDPs) and Charged Polymer Encapsulated Drug Particles (CPEDPs).

MSF5, a PEDPs, were made by first creating a dispersed phase (DP) premix by dissolving 304.7 mg of PLGA 5050 and 203 mg of sirolimus in 2.74 g of dichloromethane (DCM). Next a continuous phase (CP) premix was made by dissolving 1.08 g of Mowiol 4-88 (polyvinyl alcohol, 88% hydrolyzed) with heating to 60° C. in 58.7 g of purified water. Once the Mowiol 4-88 was fully dissolved the solution was allowed to cool and 180 mg of DCM was added to it. Next the DP was added into the CP while stirring the CP using a rotor-stator mixer to create an emulsion. Then the emulsion was added to 140 mL of purified water to precipitate the PEDPs from the emulsion. After precipitation the mixture was centrifuged to collect the PEDPs in the bottom of the centrifuge tube. Next the supernatant was poured off and the PEDPs were dispersed in a 250 mL of purified water and centrifuged again to wash the PEDPs. Next the wash supernatant was poured off and the PEDPs were dispersed in a small amount of water and vacuum dried overnight.

MSF4, a CPEDPs, were made by first creating a dispersed phase (DP) premix by dissolving 507.8 mg of PLGA 7525, 101.6 mg of Eudragit E, and 406.3 mg of sirolimus in 5.48 g of dichloromethane (DCM). Next a continuous phase (CP) premix was made by dissolving 0.585 g of Mowiol 4-88 (polyvinyl alcohol, 88% hydrolyzed) with heating to 60° C. in 31.8 g of purified water. Once the Mowiol 4-88 was fully dissolved the solution was allowed to cool and 98 mg of DCM was added to it. Next the DP was added into the CP while stirring the CP using a rotor-stator mixer to create an emulsion. Then the emulsion was added to 280 mL of purified water to precipitate the CPEDPs from the emulsion. After precipitation the mixture was centrifuged to collect the CPEDPs in the bottom of the centrifuge tube. Next the supernatant was poured off and the CPEDPs were dispersed in a 250 mL of purified water and centrifuged again to wash the CPEDPs. Next the wash supernatant was poured off and the CPEDPs were dispersed in a small amount of water and vacuum dried overnight.

MSF12, a CPEDPs, were made by first creating a dispersed phase (DP) premix by dissolving 464 mg of PLGA 7525, 166 mg of PLGA 5050, 50.0 mg of 1,2 disteroyl-sn-glycero-3-phosphatidylcholine, 20 mg of butylated hydroxytoluene, and 300.0 mg of sirolimus in 5.40 g of dichloromethane (DCM). Next a continuous phase (CP) premix was made by dissolving 0.576 g of Mowiol 4-88 (polyvinyl alcohol, 88% hydrolyzed) with heating to 60° C. in 31.3 g of purified water. Once the Mowiol 4-88 was fully dissolved the solution was allowed to cool and 96 mg of DCM was added to it. Next the DP was added into the CP while stirring the CP using a rotor-stator mixer to create an emulsion. Then the emulsion was added to 320 mL of purified water to precipitate the CPEDPs from the emulsion. After precipitation the mixture was centrifuged to collect the CPEDPs in the bottom of the centrifuge tube. Next the supernatant was poured off and the CPEDPs were dispersed in a 250 mL of purified water and centrifuged again to wash the CPEDPs. Next the wash supernatant was poured off and the CPEDPs were dispersed in a small amount of water and vacuum dried overnight.

MSF12, a CPEDPs, were made by first creating a dispersed phase (DP) premix by dissolving 464 mg of PLGA 7525, 166 mg of PLGA 5050, 50.0 mg of 1,2 disteroyl-sn-glycero-3-phosphatidylcholine, 20 mg of butylated hydroxytoluene, and 300.0 mg of sirolimus in 5.40 g of dichloromethane (DCM). Next a continuous phase (CP) premix was made by dissolving 0.576 g of Mowiol 4-88 (polyvinyl alcohol, 88% hydrolyzed) with heating to 60° C. in 31.3 g of purified water. Once the Mowiol 4-88 was fully dissolved the solution was allowed to cool and 96 mg of DCM was added to it. Next the DP was added into the CP while stirring the CP using a rotor-stator mixer to create an emulsion. Then the emulsion was added to 320 mL of purified water to precipitate the CPEDPs from the emulsion. After precipitation the mixture was centrifuged to collect the CPEDPs in the bottom of the centrifuge tube. Next the supernatant was poured off and the CPEDPs were dispersed in a 250 mL of purified water and centrifuged again to wash the CPEDPs. Next the wash supernatant was poured off and the CPEDPs were dispersed in a small amount of water and vacuum dried overnight.

MSF13, a CPEDP, were made by first creating a dispersed phase (DP) premix by dissolving 480 mg of PLGA 7525, 180 mg of PLGA 5050, 20.0 mg of 1,2 disteroyl-sn-glycero-3-phosphatidylcholine, 20 mg of butylated hydroxytoluene, and 300.0 mg of sirolimus in 5.40 g of dichloromethane (DCM). Next a continuous phase (CP) premix was made by dissolving 0.576 g of Mowiol 4-88 (polyvinyl alcohol, 88% hydrolyzed) with heating to 60° C. in 31.3 g of purified water. Once the Mowiol 4-88 was fully dissolved the solution was allowed to cool and 96 mg of DCM was added to it. Next the DP was added into the CP while stirring the CP using a rotor-stator mixer to create an emulsion. Then the emulsion was added to 320 mL of purified water to precipitate the CPEDPs from the emulsion. After precipitation the mixture was centrifuged to collect the CPEDPs in the bottom of the centrifuge tube. Next the supernatant was poured off and the CPEDPs were dispersed in a 250 mL of purified water and centrifuged again to wash the CPEDPs. Next the wash supernatant was poured off and the CPEDPs were dispersed in a small amount of water and vacuum dried overnight.

Wet coating process of MSF13 with 1,2 disteroyl-sn-glycero-3-phosphatidylcholine, DMAEMA, Eudragit E. After drying MSF13 were added to a vial that contained 6 mm yttrium stabilized zirconium cylindrical beads. The vial was placed on a 30° tilted roller mill for 15 minutes to break up aggregates of MSF13. Next a 2 mg/mL solution was made by adding the coating material (1,2 disteroyl-sn-glycero-3-phosphatidylcholine, DMAEMA, Eudragit E) into a separate vial and dissolving it in a 4/96 ethanol/cyclopentane solvent. Then the solution was added to the vial containing MSF13 and was placed back on the 30° tilted roller mill without a cap and rotated till all the solvent evaporated. The coated MSF13 CPEDPs were collected for further testing.

MSF14, a CPEDP, were made by first creating a dispersed phase (DP) premix by dissolving 464 mg of PLGA 7525, 166 mg of PLGA 5050, 50.0 mg of 1,2 disteroyl-sn-glycero-3-phosphatidylcholine, 20 mg of butylated hydroxytoluene, and 300.0 mg of sirolimus in 5.40 g of dichloromethane (DCM). Next a continuous phase (CP) premix was made by dissolving 0.576 g of Mowiol 4-88 (polyvinyl alcohol, 88% hydrolyzed) with heating to 60° C. in 31.3 g of purified water. Once the Mowiol 4-88 was fully dissolved the solution was allowed to cool and 96 mg of DCM was added to it. Next the DP was added into the CP while stirring the CP using a rotor-stator mixer to create an emulsion. Then the emulsion was added to 320 mL of purified water to precipitate the CPEDPs from the emulsion. After precipitation the mixture was centrifuged to collect the CPEDPs in the bottom of the centrifuge tube. Next the supernatant was poured off and the CPEDPs were dispersed in a 250 mL of purified water and centrifuged again to wash the CPEDPs. Next the wash supernatant was poured off and the CPEDPs were dispersed in a small amount of water and vacuum dried overnight.

Dry coating process of MSF14 with 1% 1,2 disteroyl-sn-glycero-3-phosphatidylcholine. After drying 400 mg of MSF14 were added to a vial that contained 11 grams of 6 mm yttrium stabilized zirconium cylindrical beads and 4 mg of 1,2 distearoyl-sn-glycero-3-phosphatidylcholine (1,2 disteroyl-sn-glycero-3-phosphatidylcholine). The vial was placed on a roller mill for 2 hours to mechanically coat the outside of MSF14.

Wet coating process of MSF14 with 1% 1,2 disteroyl-sn-glycero-3-phosphatidylcholine. After drying 400 mg MSF14 were added to a vial that contained 6 mm yttrium stabilized zirconium cylindrical beads. The vial was placed on a 300 tilted roller mill for 15 minutes to break up any aggregates of MSF14. Next a 2 mg/mL solution was made by adding 4 mg of 1,2 disteroyl-sn-glycero-3-phosphatidylcholine into a separate vial and dissolving it in 2 mL of a 4/96 ethanol/cyclopentane solvent. Then the solution was added to the vial containing MSF14 and was placed back on the 30° tilted roller mill without a cap and rotated till all the solvent evaporated. The coated MSF14 CPEDPs were collected for further testing.

MSF25, a CPEDP, was made by first creating a dispersed phase (DP) premix by dissolving 360 mg of PLGA 7525, 2,200 mg of PLGA 8515, 160.0 mg of 1,2 disteroyl-sn-glycero-3-phosphatidylcholine, 80 mg of butylated hydroxytoluene, and 1,320.0 mg of sirolimus in 50.55 g of dichloromethane (DCM). Next a continuous phase (CP) premix was made by dissolving 1.312 g of Mowiol 4-88 (polyvinyl alcohol, 88% hydrolyzed) with heating to 60° C. in 162.19 g of purified water. Once the Mowiol 4-88 was fully dissolved the solution was allowed to cool and 492 mg of DCM was added to it. Next the DP was added into the CP while stirring the CP using a rotor-stator mixer to create an emulsion. Then the emulsion was added to 1148 mL of purified water to harden the CPEDPs. After hardening the mixture was centrifuged to collect the CPEDPs in the bottom of the centrifuge tube. Next the supernatant was poured off and the CPEDPs were dispersed in a 1148 mL of purified water and centrifuged again to wash the CPEDPs. Next the wash supernatant was poured off and the CPEDPs were dispersed in a small amount of water and vacuum dried overnight.

### Zeta Potential of Polymer Encapsulated Drug Particles (PEDPs) and Charged Polymer Encapsulated Drug Particles (CPEDPs).

The zeta potential of the PEDPs and CPEDPs was measured using a Beckman Coulter Delsa Nano Particle Analyzer to characterize the zeta potential via electrophoretic light scattering (ELS) of MSF4, MSF5, and MSF12 as it is desired to have positive surface charge for particle adhesion to live tissue. Samples were created by dispersing 8-12 mg of each type of PEDP or CPADP in approximately 1 mL of purified water using an ultrasound bath for the dispersion process. The zeta potential measurements were then conducted by depositing a few drops of dispersion in a clean flow though cell with 25° C. purified water prior to measurement. The zeta potential measurements can be seen in Table 8.

TABLE 8
Zeta potential measurements.

| | | Zeta |
|---|---|---|
| Sample | PEDP and CPEDP Configuration | Potential |
| MSF5 | Nonionic Ingredients: PLGA 5050 with | −19.9 |
| | Sirolimus Drug | |
| MSF4 | Eudragit E Cationic polymer mixed with | +14.69 |
| | PLGA 7525 and Sirolimus Drug | |
| MSF12 | 5% Zwitter-ionic phosphatidylcholine | +7.35 |
| | mixed with PLGA 7525 and PLGA 5050 | |
| MSF13 | 2% 1,2 disteroyl-sn-glycero-3- | +5.47 |
| | phosphatidylcholine, 48% PLGA 7525, | |
| | 18% PLGA 5050, 2% BHT, 30% | |
| | Sirolimus Drug | |
| MSF13 + 1,2 disteroyl-sn-glycero-3- | MSF13 with outside coating of zwitter- | +7.35 |
| phosphatidylcholine coating, 1% of | ionic phosphatidylcholine using wet | |
| microsphere mass | coating process | |
| MSF13 + DMAEMA coating, 1% of | MSF13 with outside coating of cationic | +35.26 |
| microsphere mass | polymer using wet coating process | |
| MSF13 + Eudragit E coating, 1% of | MSF13 with outside coating of cationic | +34.02 |
| microsphere mass | polymer using wet coating process | |
| MSF14 | 2% 1-palmitoyl-2-stearoyl-sn-glycero-3- | −13.34 |
| | phosphocholine, 56% PLGA 7525, 10% | |
| | PLGA 5050, 2% BHT, 30% Sirolimus | |
| | Drug | |
| MSF14 + 1,2 disteroyl-sn-glycero-3- | MSF14 with outside coating of zwitter- | −2.09 |
| phosphatidylcholine coating, 1% of | ionic phosphatidylcholine using dry | |
| microsphere mass | coating process | |
| MSF14 + 1,2 disteroyl-sn-glycero-3- | MSF14 with outside coating of zwitter- | +1.57 |
| phosphatidylcholine coating, 1% of | ionic phosphatidylcholine using wet | |
| microsphere mass | coating process | |
| MSF25 no Drug or DSPC | 82.0% PLGA 8515, 16.0% PLGA 7525, | NA |
| | 2% BHT | |
| MSF25 no DSPC | 56.9% PLGA 8515, | NA |
| | 11.1% PLGA 7525, 2% BHT, 30% | |
| | Sirolimus Drug | |
| MSF25-5 | 4% 1,2 disteroyl-sn-glycero-3- | NA |
| | phosphatidylcholine, 55% PLGA 8515, 9% | |
| | PLGA 7525, 2% BHT, 30% Sirolimus | |
| | Drug | |
| MSF25-5 + 1,2 disteroyl-sn- | MSF25-5 with outside coating of | NA |
| glycero-3-phosphatidylcholine | zwitterionic phosphatidylcholine using wet | |
| coating, 3% of microsphere mass | coating process | |

Porcine Animal Study with Formulation MS122, MS123, and MS124.

Formula MS122 Sirolimus Coating Solution Preparation. Coating Solution Formula MS122 a charged polymer encapsulated drug particle (CPEDP) aqueous formulation was made. First MSF13, a CPEDPs, was made using the process described above. Then MSF13 was coated with 1% Eudragit E using the wet coating process described above. Next 313 mg of the coated MSF13 was added to a vial and dispersed in 2.06 mL of purified water. This solution was sonicated for 5-10 minutes to fully disperse the CPEDPs. Then in a separate vial a premix of 301 mg of 1-palmitoyl-2-lauroyl-sn-glycero-3-phosphocholine and 32 mg of 1,2-dilauroyl-sn-glycero-3-phosphatidylcholine was dissolved in 1.37 mL ethanol. Once dissolved 2.06 mL of water was added to dilute the premix. Next the premix was added to the dispersed CPEDPs and sonicated for 5-10 minutes. This solution, Formula MS122, was used to coat balloons for drug transfer testing in pigs.

Formula MS123 Sirolimus Coating Solution Preparation. Coating Solution Formula MS123 a charged polymer encapsulated drug particle (CPEDP) aqueous formulation was made. First MSF13, a CPEDPs, was made using the process described above. Then MSF13 was coated with 1% 1,2 disteroyl-sn-glycero-3-phosphatidylcholine using the wet coating process described above. Next 392 mg of the coated MSF13 was added to a vial and dispersed in 2.44 mL of purified water. This solution was sonicated for 5-10 minutes to fully disperse the CPEDPs. Then in a separate vial a premix of 333 mg of 1-palmitoyl-2-lauroyl-sn-glycero-3-phosphocholine and 38 mg of 1,2-dilauroyl-sn-glycero-3-phosphatidylcholine was dissolved in 1.62 mL ethanol. Once dissolved 2.44 mL of water was added to dilute the premix. Next the premix was added to the dispersed CPEDPs and sonicated for 5-10 minutes. This solution, Formula MS123, was used to coat balloons for drug transfer testing in pigs.

Formula MS124 Sirolimus Coating Solution Preparation. Coating Solution Formula MS124 a charged polymer encapsulated drug particle (CPEDP) and crystalline drug particle aqueous formulation was made. First MSF13, a CPEDPs, was made using the process described above. Then MSF13 was coated with 1% 1,2 disteroyl-sn-glycero-3-phosphatidylcholine using the wet coating process described above. Next 124 mg of crystalline sirolimus was added to a vial with 2.44 mL of purified water. Then a sonic probe was used to conduct a particle size reduction on the crystalline drug. Next 196 mg of the coated MSF13 was added to the vial containing the crystalline sirolimus particles. Then in a separate vial a premix of 194 mg of 1-palmitoyl-2-lauroyl-sn-glycero-3-phosphocholine and 136 mg of 1,2-dilauroyl-sn-glycero-3-phosphatidylcholine was dissolved in 1.61 mL ethanol. Once dissolved 2.44 mL of water was added to dilute the premix. Next the premix was added to the dispersed CPEDPs and sonicated for 5-10 minutes to fully disperse the CPEDPs and the crystalline sirolimus particles. This solution, Formula MS124, was used to coat balloons for drug transfer testing in pigs.

Formulations MS122, MS123, and MS124 were uniformly coated onto balloons of various sizes for acute drug transfer testing in a pig. The balloons were coated using an automated coating machine that precisely dispensed a prespecified volume of coating solution onto the surface of the balloon. The prespecified volume of solution was calculated based on the coating solution concentration and the desired nominal drug dose per balloon. The balloon sizes, diameter×length, were 6×30, and 18×65. The amount of drug per square millimeter for each balloon was 3.0 for the 18 mm diameter balloons. The amount of drug per square millimeter for each balloon was 3.5 for the 6 mm diameter balloons. This gave nominal drug dosing of 906 μg, and 11358 μg for the respective 6.0 and 18 mm diameter balloons. The 6 mm balloons were used in the pig's urethra with a stretch ratio of 1.0 to 2.0. Lastly the 18 mm balloons were used in the pig's esophagus, small intestine, and colon with stretch ratios of 1.0 to 2.0. For the urological and gastrointestinal treatments the balloon was tracked into position and the coating was allowed to hydrate for 1 minute prior to inflation. For the gastrointestinal treatments a gastroscope, enteroscope, or colonoscope was used to visualize the treatment site and flush the wall of the treatment site prior to use of the DCB. After all treatments the pig was survived for 24 hours then the treated tissue was excised in the necropsy lab and assayed for drug content. The measured sirolimus drug concentration in the various tissues can be seen in Table 9. The residual amount of drug left on the balloons post treatment can be seen in Table 10.

TABLE 9
Formulations MS122, MS123, and MS124 measured
drug concentrations in various tissues

| Sample | Formula | Sirolimus [ug/g] |
|---|---|---|
| Prox Urethra | MS122 | 2.200 |
| Mid Urethra | MS123 | 0.375 |
| Dist Urethra | MS124 | 1.690 |
| Prox Esophagus | MS122 | 0.127 |
| Mid Esophagus | MS124 | 0.104 |
| Dist Esophagus | MS123 | Below limit of quantification |
| Prox Duodenum | MS122 | Below limit of quantification |
| Mid Duodenum | MS123 | 10.400 |
| Dist Duodenum | MS124 | Below limit of quantification |
| Prox Colon | MS124 | 9.470 |
| Dist Colon | MS123 | 8.230 |

TABLE 10
Residual amount of drug left on the balloons
post treatment for MSF122, MSF123, and MS124.

| Sample | Formula_Balloon | Sirolimus [ug] | % of Dose |
|---|---|---|---|
| Dist Urethra | MS124-6x50-2 | 1372.79 | 40.4 |
| Prox Colon | MS124-18x65-1 | 6201.47 | 54.6 |
| Dist Duodenum | MS124-18x65-4 | 113.58 | 1.0 |
| Mid Esophagus | MS124-18x65-3 | 1453.82 | 12.8 |
| Mid Urethra | MS123-6x50-3 | 683.00 | 20.1 |
| Dist Esophagus | MS123-18x65-2 | 2419.25 | 21.3 |
| Mid Duodenum | MS123-18x65-3 | 2226.17 | 19.6 |
| Distal Colon | MS123-18x65-1 | 6485.42 | 57.1 |
| Prox Urethra | MS122-6x50-2 | 1077.17 | 31.7 |
| Prox Duodenum | MS122-18x65-3 | 34.07 | 0.3 |
| Prox Esophagus | MS122-18x65-2 | 147.65 | 1.3 |

Formula C6SsusF9 Sirolimus Coating Solution Preparation. Coating Solution Formula C6SsusF9 a charged polymer encapsulated drug particle (CPEDP) dispersed in predominantly nonpolar organic solvent with dissolved phospholipids was made. First MSF18, a CPEDP, was made using the same process described above to make MSF13 and MSF14. Then 328 mg of dried MSF18 was coated with 9.84 mg (3% of CPEDPs) 1,2-distearoyl-sn-glycero-3-phosphocholine using the wet coating process described above. Then in a separate vial 328 mg of 1-palmitoyl-2-stearoyl-sn-glycero-3-phosphocholine was dissolved in 5 mL of 4/96 ethanol/cyclohexane v/v solvent. Once dissolved this solution was added to the vial containing the coated CPEDPs. Next the full mixture was a sonicated for 2-5 minutes to fully disperse the CPEDPs. This solution, C6SsusF9, was collected and assayed for drug concentration. The resulting measurement was 17.5 mg/mL sirolimus.

Formula C6SsusF11 Sirolimus Coating Solution Preparation. Coating Solution Formula C6SsusF11, a polymer encapsulated drug particle (CPEDP) dispersed in nonpolar organic solvent with dissolved mismatched, unsaturated acyl group phospholipids, was made. First, MSF21, a CPEDP, was made using the same process described above to make MSF13 and MSF14. Then 303 mg of MSF21 was weighed into a vial and 2 mL of cyclohexane was added to it. The contents of the vial were sonicated to disperse the CPEDPs to create premix 1. Then in a separate vial 303 mg of 1-palmitoyl-2-oleoyl-sn-glycero-3-phosphocholine was dissolved with mild heat in 2 mL cyclohexane solvent to create premix 2. Next premix 2 was added to premix 1 and the full mixture was a sonicated for 2-5 minutes to fully disperse the CPEDPs. This solution, C6SsusF11, was collected and assayed for drug concentration. The resulting measurement was 20.0 mg/mL sirolimus.

Formula C6SsusF12 Sirolimus Coating Solution Preparation. Coating Solution Formula C6SsusF12, a polymer encapsulated drug particle (CPEDP) dispersed in nonpolar organic solvent with dissolved matched, unsaturated acyl group phospholipids, was made. First, MSF21, a CPEDPs, was made using the same process described above to make MSF13 and MSF14. Then 302 mg of MSF21 was weighed into a vial and 2 mL of cyclohexane was added to it. The contents of the vial were sonicated to disperse the CPEDPs to create premix 1. Then in a separate vial 304 mg of 1,2-dioleoyl-sn-glycero-3-phosphocholine was dissolved with mild heat in 2 mL cyclohexane solvent to create premix 2. Next premix 2 was added to premix 1 and the full mixture was a sonicated for 2-5 minutes to fully disperse the CPEDPs. This solution, C6SsusF12, was collected and assayed for drug concentration. The resulting measurement was 20.6 mg/mL sirolimus.

Formula C6SsusF13 Sirolimus Coating Solution Preparation. Coating Solution Formula C6SsusF13, a polymer encapsulated drug particle (CPEDP) dispersed in nonpolar organic solvent with dissolved matched, unsaturated acyl group phospholipids, was made. First, MSF21, a CPEDP, was made using the same process described above to make MSF13 and MSF14. Then 304 mg of MSF21 was weighed into a vial and 2 mL of cyclohexane was added to it. The contents of the vial were sonicated to disperse the CPEDPs to create premix 1. Then in a separate vial 30.3 mg of 1,2-dioleoyl-sn-glycero-3-phosphocholine and 272.7 mg of 1-palmitoyl-2-oleoyl-sn-glycero-3-phosphocholine was dissolved with mild heat in 2 mL cyclohexane solvent to create premix 2. Next premix 2 was added to premix 1 and the full mixture was a sonicated for 2-5 minutes to fully disperse the CPEDPs. This solution, C6SsusF12, was collected and assayed for drug concentration. The resulting measurement was 20.1 mg/mL sirolimus.

Formula C6SsusF13-3, MSF25 Sirolimus Coating Solution Preparation. Coating Solution Formula C6SsusF13-3, a polymer encapsulated drug particle (CPEDP) dispersed in nonpolar organic solvent with dissolved matched, unsaturated acyl group phospholipids, was made. First, MSF25, a CPEDP, was made using the methods described above. Then 2.622 g of MSF25 was weighed into a bottle and 22.5 mL of cyclohexane was added to it. The contents of the vial were sonicated to disperse the CPEDPs to create premix 1. Then in a separate vial 262.2 mg of 1,2-dioleoyl-sn-glycero-3-phosphocholine and 2.360.1 g of 1-palmitoyl-2-oleoyl-sn-glycero-3-phosphocholine was dissolved with mild heat in 22.5 mL cyclohexane solvent to create premix 2. Next premix 2 was added to premix 1 and the full mixture was a sonicated for 2-5 minutes to fully disperse the CPEDPs. This solution, C6SsusF13-3, was collected and assayed for drug concentration. The resulting measurement was 17.98 mg/mL sirolimus. 18 mm diameter by 65 mm length balloon catheters of were coated using this formulation. The target dose for the catheters was 9.2 mg. The balloons were pleat and folded, packaged, vacuum dried, and sterilized. These balloons were used to treat 6 pigs in their esophagus, duodenum, and colon detailed in Example IV-4 below.

### Part II. Human Clinical Testing Urethral Strictures.

Uroflowmetry (Qₘₐₓ measurement). Uroflowmetry is performed by urinating into a special urinal, toilet, or disposable device that has a measuring device built into it. The parameter, Qₘₐₓ, is the maximum flow rate measured during a uroflowmetery test. This method was used prior to treatment (baseline) and at follow-up visits of 14 days, 1, 3, 6, 12 months, and 2 years to demonstrate the longevity of the treatment.

Post-void residual (PVR) is a measurement of the volume of urine left in the bladder after voiding. It is measured using ultrasound prior to treatment (baseline) and at follow-up visits of 14 days, 1, 3, 6, 12 months, and 2 years to demonstrate the longevity of the treatment.

International Prostate Symptom Score (IPSS). The IPSS is based on the answers to eight questions-seven regarding disease symptoms and one question related to the patient's quality of life: 1) Incomplete Emptying; How often have you had the sensation of not emptying your bladder? 2) Frequency; How often have you had to urinate less than every two hours? 3) Intermittency; How often have you found you stopped and started again several times when you urinated? 4) Urgency; How often have you found it difficult to postpone urination? 5) Weak Stream; How often have you had a weak urinary stream? 6) Straining; How often have you had to strain to start urination? 7) Nocturia; How many times did you typically get up at night to urinate? 8) Quality of Life Due to Urinary Symptoms; If you were to spend the rest of your life with your urinary condition just the way it is now, how would you feel about that? Although the IPSS was developed for BPH it can be applied to other bladder outlet obstructive diseases such as stricture to determine if obstructive symptoms are improved after a medical treatment. For the symptom questions, the patient is asked to choose the rating that best represents their condition. The scale ranges from 0 to 5, with 5 representing the most symptomatic disease. The seven symptom scores are summed to give an overall maximum possible score of 35. The answer to the quality of life question is scored on a scale of 0 to 6. According to these scoring systems, the scores can be categorized as follows: symptoms are mild if the score is 7 or less; symptoms are moderate if the score is 8 to 19; and symptoms are severe if the score is 20 to 35. This questionnaire was given prior to treatment (baseline) and at follow-up visits of 14 days, 1, 3, 6, 12 months, and 2 years to demonstrate the longevity of the treatment.

In this part, the drug coating on the drug-coated balloon catheter included paclitaxel and an excipient as a homogeneous blend. The excipient was pentaerythritol ethoxylate (PEE) 15/4. The weight ratio of the excipient to paclitaxel in the coating was 1:3.

### Example II-1. Testing of Drug-Coated Balloons in Urethral Strictures of Human Subjects

Drug-coated balloon catheters with a dose density of 3.5 μg of paclitaxel per millimeter squared of balloon surface area were used to treat human subjects that had stricture disease in a clinical study. The drug-coated balloon catheters had nominal diameters of 6, 8, 10, 12, and 14 mm and lengths of 30 and 50 mm at nominal pressure of 6 atm. The paclitaxel (PTX) dosing per balloon size can be seen in Table 11.

TABLE 11
Paclitaxel (PTX) dosing per balloon size.

| Diameter (mm) | 30 mm Length | 50 mm Length |
|---|---|---|
| 6 | 1979 μg PTX | 3299 μg PTX |
| 8 | 2639 μg PTX | 4398 μg PTX |
| 10 | 3299 μg PTX | 5498 μg PTX |
| 12 | 3958 μg PTX | 6597 μg PTX |
| 14 | 4618 μg PTX | 7697 μg PTX |

The drug-coated balloon catheters had a dual lumen shaft design with a single inflatable balloon. One lumen was sized to accommodate a 0.038″ guide wire lumen. The other lumen was the inflation port lumen and allows the balloon to be inflated with mixture of saline and contrast fluid. The drug-coated balloon catheter had a manifold with two Luer-style connections, one connection was compatible with an inflation syringe, the other allowed the guidewire to protrude out of the manifold so the balloon catheter could freely slide onto the guidewire. The 6 and 8 mm drug-coated balloon catheters had a rated burst pressure of 12 atmospheres. The 10, 12, and 14 mm drug-coated balloon catheters had a rated burst pressure of 10 atmospheres. The balloon was made of polyamide.

Treatments were performed for 53 patients for bulbar urethral strictures. Of these patients, 5 were retreated due to stricture recurrence, bring the total number of treatments to 58. All patients were male as the study excluded female patients. Subjects enrolled in the study had a minimum of 1 and a maximum of 3 prior interventions for urethral stricture. The age range was 50.7±15.47 years. The etiologies of urethral stricture across the patient population were 50.9% traumatic, 45.3% iatrogenic, and 3.8% idiopathic. Of 53 patients, 7 had a suprapubic catheter at baseline. The stricture length on average was 0.9 cm and the average stricture diameter was 2.47 mm.

The clinical treatment strategy involved predilation of the stricture with direct vision internal urethrotomy (DVIU), an uncoated non-compliant balloon, or a combination of both. An uncoated balloon was used to predilate 32 patients, 16 patients were predilated with both an uncoated balloon and DVIU, and 10 patients were predilated with DVIU only. Following predilation all subjects were treated with the paclitaxel drug-coated balloons. 26 patients were treated with the 8×30 mm balloons. 27 patients were treated with the 10×30 mm balloons.

Clinical subjects were evaluated at 14, 30, 90, 180, 365 days, and 2 years after the index procedure. Evaluations included analysis of stricture free rate, uroflowmetry including Qₘax, and PVR. Additionally, pharmacokinetic analysis was completed for the paclitaxel content in the blood, urine, semen, and residual drug on the drug-coated balloons.

On average patient IPSS improved from a baseline average of 25.2, to 5.2 at 14 days, 4.3 at 30 days, 6.1 at 90 days, 4.6 at 180 days, 4.5 at 365 days, and 6.9 at 2 years. Average patient Qₘₐₓ at baseline was 5.0 mL/sec and improved to 22.2 mL/sec at 14 days, 22.8 mL/sec at 30 days, 21.4 mL/sec at 90 days, 19.8 mL/sec at 180 days, 20.1 mL/sec at 365 days, and 17.5 mL/sec at 2 years. Average patient PVR was 141.4 mL at baseline and improved to 35.7 mL at 14 days, 36.1 mL at 30 days, 38.8 mL at 90 days, 30.1 mL at 180 days, 24.6 mL at 365 days, and 45.5 mL at 2 years. Stricture free rate was 75.5% (37/49) at day 180 and 74.5% (37/47) at 365 days.

Human plasma paclitaxel concentration on average was 0.1 ng/mL immediately post treatment and was below the level of quantification for all other later time points, 1 hour, 3 hours, 5 hours, 10 hours, 24 hours, and 5 days. Human urine paclitaxel concentration on average was 184 ng/mL immediately post procedure, 2.6 ng/mL at 5 days, 0.3 ng/mL at 14 days, and 0.1 ng/mL at 30 days. Human semen paclitaxel concentration on average was 2.5 ng/mL at 14 days and 1.0 ng/mL at 30 days.

Residual drug content on the balloons used to treat human subject was on average 2.7% of the original dose with a range of 0.1% to 28.0%.

### Example II-2. Human Clinical Subject B from Example II-1 Treated with an Uncoated Predilation Balloon Followed by a 10 mm Nominal Diameter Drug-Coated Balloon Catheter

Subject B had a 1.8 cm length by 2.0 mm diameter stricture in his anterior urethra. Specifically, the bulbar portion of the anterior urethra. This was determined by conducting a retrograde urethragram. The human clinical subject had a baseline Qₘₐₓ of 8 mL/second, PVR of 45 mL, and a baseline IPSS score of 30. First a cystoscope was inserted into the urethra. Then a guidewire was inserted into the working channel of the cystoscope. Next a predilation balloon that had a nominal diameter of 10 mm and a length of 20 mm was inserted into the urethra over the guidewire and positioned so the balloon crossed the stricture. The predilation balloon was inflated to 20 atmospheres with a syringe that had a pressure gauge on it. The syringe contained a mixture of saline and contrast media. A fluoroscopic image was acquired to ensure the balloon had a uniform expansion. Once this was confirmed the balloon was deflated and withdrawn from the urethra. Next a drug-coated balloon that had a nominal diameter of 10 mm and a length of 30 mm was inserted into the urethra over the guidewire. The drug-coated balloon was positioned such that the balloon body completely covered the predilated stricture area. The drug-coated balloon was held in position for at least 1 minute prior to inflating to hydrate the coating. Then the drug-coated balloon was inflated with a mixture of saline and contrast media using the syringe that had a pressure gauge on it. The balloon was inflated to 10 atmospheres for 5 minutes. Then the balloon was deflated and withdrawn from the human subject. The stretch ratio for the 10 mm nominal diameter drug-coated balloon was 1.3 to 1.5. The diameter of the dilated stricture was 10 mm after dilation. The residual drug remaining on the balloon after use was analyzed. The residual amount of paclitaxel left on the balloon was 49.1 μg (1.5 percent of the initial drug load). The human clinical subject had follow-up visits at 14, 30, 90, 180, 365 days, and 2 years to measure maximum urine flow rate, PVR, and IPSS score. Additionally, the urethral caliber of the human clinical subject was assessed at 6 and 12 months to determine if the urethra was greater than 16 French (5.3 mm) by visualizing and passing a flexible cystoscope past the previously treated area. The human clinical subject had a maximum urine flow rate improvement from 8 mL/second to 25, 29, 36, 34, 34, and 23 mL/second at follow-up visits of 14, 30, 90, 180, 365 days, and 2 years respectively. The human clinical subject had PVR improvement from 45 mL to 30, 28, 35, 26, 3, and 10 mL at follow-up visits of 14, 30, 90, 180,365 days, and 2 years respectively. The human clinical subject had an IPSS improvement from 30 to 4, 3, 5, 2, 3, and 3 at follow-up visits of 14, 30, 90, 180 365 days, and 2 years respectively. The human clinical subject had a urethra caliber greater than 16 French (5.3 mm) at 6 and 12 months.

### Part III. Human Clinical Testing Benign Prostatic Hyperplasia.

In this Part, the drug coating on the drug-coated balloon included paclitaxel and an excipient as a homogeneous blend. The excipient was pentraerythritol ethoxlate (PEE) 15/4. The weight ratio of the excipient to paclitaxel in the coating was 1:3.

### Example III-1. BPH Human Clinical Testing: Single Lumen & Reinforced Fixed Wire Drug-Coated Balloon Catheter

Drug-coated balloon catheters with a dose density of 2.4 μg of paclitaxel per millimeter squared were used to treat human subjects that had benign prostatic hyperplasia disease in a clinical study. The balloons had a single neck positioned approximately 10 mm from the distal cone of the balloon. The neck length was 2 mm for all the balloons. The proximal lobe or treatment lobe had a range of different diameters and length, while the distal lobe or bladder lobe was always the same 10 mm length and matched the diameter of the treatment lobe. The drug-coated balloon catheters had nominal diameters of 30, 35, 40, and 45 mm and dilation lengths of 30, 35, 40, 45, and 50 mm at a nominal inflation pressure of 2 atm. For balloon diameters of 30 mm the neck diameter was 12 mm, for balloon diameters of 35 mm the neck diameter was 15 mm, for balloon diameters of 40 mm the neck diameter was 20 mm, and for balloon diameters of 45 mm the neck diameter was 23 mm. All balloons sizes had a rated burst pressure of 4 atm. The balloons were coated with paclitaxel from halfway up the proximal cone, over the entire treatment lobe, over the entire neck section, and the body portion of the bladder lobe. The paclitaxel (PTX) dosing per balloon size can be seen in Table 12.

TABLE 12
Paclitaxel (PTX) dosing per balloon size.

| Diameter | 30 mm | 35 mm | 40 mm | 45 mm | 50 mm |
|---|---|---|---|---|---|
| (mm) | Length | Length | Length | Length | Length |
| 30 | 9586 μg | 11433 μg | 12567 μg | 13661 μg | 15256 μg |
| | PTX | PTX | PTX | PTX | PTX |
| 35 | 12403 μg | 13915 μg | 15238 μg | 16561 μg | 18073 μg |
| | PTX | PTX | PTX | PTX | PTX |
| 40 | 15220 μg | 16543 μg | 18055 μg | 19567 μg | 20890 μg |
| | PTX | PTX | PTX | PTX | PTX |
| 45 | 18037 μg | 19455 μg | 20872 μg | 22290 μg | 23707 μg |
| | PTX | PTX | PTX | PTX | PTX |

Two catheter designs were used in this study. The balloon shape, material, and dimensions were identical across the two catheter platforms. The difference between the two balloon catheters was the catheter shaft design. The first design was a single lumen design without a Luer hub and was designed to be back loaded into a cystoscope. The second design was a reinforce fixed wire catheter shaft with a Luer hub attached and was designed to be positioned side-by-side with a cystoscope.

Single lumen design: The drug-coated balloon catheters had a single lumen nylon 12 shaft design with holes punched under the balloon to allow inflation of the drug-coated balloon. The catheter shaft did not have a Luer and was designed to connect to a Tuohy Borst valve and Luer compatible stopcock after passing through the working channel of a cystoscope. The balloon neck was reinforced with ultra-high molecular weight polyethylene (UHMWPE) fibers that were fixed in place to minimize diameter growth during inflation. The balloon neck anchors into the bladder neck during balloon inflation and prevents migration of the proximal balloon lobe into the bladder. The proximal lobe of the balloon (treatment lobe) is located in the prostatic urethra and sized to fit the prostate. The distal balloon lobe is used for positioning and provides dilation of the bladder neck and any intra prostatic protrusion present during balloon inflation. The balloon is made of a high durometer polyether block amide (74D PEBA). At the distal end of the catheter a silicone Coude tip is attached that allows the catheter to be tracked in the prostatic urethra. The Coude tip is specially curved to conform to the male urethra anatomy and is atraumatic to prevent damage during insertion. The balloon portion of the catheter was pleat and folded down to a caliber of 19 French and a sheath was place over the balloon. The delivery sheath had three functions. One was to cover and protect the balloon. Two was to form a smooth cylindrical catheter body that allows the balloon catheter to be tracked through the urethra and positioned into the prostatic urethra. The last was to recapture the balloon after the treatment to facilitate removal of the device. Balloon catheters with a diameter of 30 or 35 mm had a 21Fr sheath while balloon catheters with a diameter of 40 or 45 mm had a 24 Fr sheath. The sheath was constructed with an inner layer of etched PTFE liner, a middle layer of a flat coiled wire, and an outer layer of polyether block amine (35D PEBA). The sheath and Coude tip were sized identically and have mating features to make a single smooth insertion surface.

Reinforce fixed wire design: The drug-coated balloon catheters had a catheter shaft that consisted of a single lumen extrusion of high durometer polyether block amide (72D PEBA). Within the extrusion lumen a cylindrical 304 stainless steel mandrel ran the length of the catheter and under the balloon. The mandrel is thermally bonded into the distal tip and the proximal end of the catheter near the Luer hub. The mandrel is reinforced under the balloon section with a 304 stainless steel tube to prevent buckling. The catheter shaft extrusion is terminated under the proximal balloon bond allowing inflation of the drug-coated balloon. At the proximal end of the catheter shaft a female Luer hub is adhesively bonded to the catheter shaft extrusion to allow connection to an inflation device. The balloon neck is reinforced with ultra-high molecular weight polyethylene (UHMWPE) fibers that were fixed in place to minimize diameter growth during inflation. The balloon neck anchors into the bladder neck during balloon inflation and prevents migration of the proximal balloon lobe into the bladder. The proximal lobe of the balloon (treatment lobe) is located in the prostatic urethra and sized to fit the prostate. The distal balloon lobe is used for positioning and provides dilation of the bladder neck and any intra prostatic protrusion present during balloon inflation. The balloon was made of a high durometer polyether block amide (74D PEBA). At the distal end of the catheter a silicone Coude tip is attached that allows the catheter to be tracked in the prostatic urethra. The Coude tip is specially curved to conform to the male urethra anatomy and is atraumatic to prevent damage during insertion. The balloon portion of the catheter was pleat and folded down to a caliber of 19 French and a sheath was place over the balloon. The delivery sheath had three functions. One was to cover and protect the balloon. Two was to form a smooth cylindrical catheter body that allows the balloon catheter to be tracked through the urethra and positioned into the prostatic urethra. The last was to recapture the balloon after the treatment to facilitate removal of the device. Balloon catheters with a diameter of 30 and 35 mm had a 21 Fr sheath while balloon catheters with a diameter of 40 and 45 mm had a 24Fr sheath. The sheath was constructed with an inner layer of etched PTFE liner, a middle layer of a flat coiled wire, and an outer layer of polyether block amine (72D PEBA). The sheath and Coude tip were sized identically and have mating features to make a single smooth insertion surface. This design also included a preloaded obturator place over the top of the catheter shaft. The obturator was made of LDPE, had a radiused distal tip, and had a flared proximal end to interface with the sheath. The combined sheath and obturator were used to track through the urethra and recapture the balloon after the treatment.

In total 80 patients were treated for benign prostatic hyperplasia. All patients were male as the study excluded female patients. Subjects enrolled in the study had a minimum IPSS score of 13, a Qₘₐₓ ranging from 5 to 15 mL, prostate volumes between 20 and 80 grams, and prostatic urethral lengths between 35 and 55 mm. The average age was 65.8±7.82 years.

The clinical treatment strategy involved predilation of the prostate to create a commissurotomy between the lateral lobes. The predilation balloon was an uncoated balloon catheter identical in size or smaller than the selected drug-coated balloon. The predilation balloons were designed identically to the drug-coated balloons. 49 patients were treated with the single lumen catheter shaft design and 31 patients were treated with the reinforced fixed wire catheter shaft design. 18 patients were treated with a 30×35 mm drug-coated balloon, 32 patients were treated with a 35×35 mm drug-coated balloon, 8 patients were treated with a 35×45 mm drug-coated balloon, and 22 patients were treated with a 40×45 mm drug-coated balloon.

Clinical subjects were evaluated at 14, 30, 90, 180, and 365 days after the index procedure. Evaluations included analysis IPSS, uroflowmetry including Qₘₐₓ, and PVR. Additionally, pharmacokinetic analysis was completed for the paclitaxel content in the blood, urine, semen, and residual drug on the drug-coated balloons.

On average patient IPSS improved from a baseline average of 22.3, to 10.7 at 14 days, 9.0 at 30 days, 8.1 at 90 days, 8.0 at 180 days, and 8.3 at 365 days. Average patient Qₘₐₓ at baseline was 10.9 mL/sec and improved to 18.5 mL/sec at 14 days, 20.1 mL/sec at 30 days, 20.4 mL/sec at 90 days, 20.1 mL/sec at 180 days, and 18.3 mL/sec at 365 days. Average patient PVR was 64.0 mL at baseline and improved to 41.4 mL at 14 days, 28.4 mL at 30 days, 33.9 mL at 90 days, 29.7 mL at 180 days, and 31.7 mL at 365 days.

Human plasma paclitaxel concentration on average was 0.2 ng/mL immediately post treatment, 0.2 ng/mL at 1 hour, 0.1 ng/mL at 3 hours, 0.1 ng/mL at 5 hours, 0.07 ng/mL at 10 hours, 0.03 ng/mL at 24 hours, and 0.02 ng/mL at 4 days. Human urine paclitaxel concentration on average was 598 ng/mL immediately post procedure, 202 ng/mL at 4 days, 5.2 ng/mL at 14 days, and 5.1 ng/mL at 30 days. Human semen paclitaxel concentration on average was 5.3 ng/mL at 14 days, 3.2 ng/mL at 30 days, and 0.12 ng/mL at 6 months.

Residual drug content on the balloons used to treat human subject was on average 23.0% of the original dose with a range of 7.7% to 47.1%.

### Part IV. Preclinical Testing Drug-Coated Balloons in the Gastrointestinal (GI) Tract.

In this Part, unless otherwise indicated, the drug coating on the paclitaxel drug-coated balloons included paclitaxel and an excipient as a homogeneous blend, with a coating dose of paclitaxel of 3.5 microgram/mm². The excipient was pentraerythritol ethoxlate (PEE) 15/4. The weight ratio of the excipient to paclitaxel in the coating was 1:3.

### Example IV-1. GI Preclinical Study 1

Twenty-eight balloon catheters (10, 15 and 18 mm in diameter and 55 mm in length) were inflated to 1 atmosphere and wiped with an ethanol wipe to clean the balloon surface. The balloons were coated using Formulation 23 from Example I-1 with sufficient coating solution to achieve 3.5 microgram paclitaxel per square mm of balloon surface. The balloons were then dried, folded, sheathed, packaged in a Tyvek pouch and ethylene oxide sterilized in preparation for animal testing.

For this study male pigs were used. Pretreatment endoscopy was conducted to measure the inner diameter of the esophagus, duodenum, and colon treatment sites before drug-coated balloon treatment. For biliary tract treatments endoscopic retrograde cholangiopancreatography (ERCP) with sphincterotomy of the ampula of vater was conducted to take a cholangiogram of the biliary tract and identify the treatment sites. The esophagus, duodenum and colon treatment site diameters were approximately 15-18 mm. The biliary tract treatment site diameters were 4-8 mm. The balloon catheters were chosen such that the stretch ratio for the treatments was approximately 1.1-2.2. Drug-coated balloon catheters were used with nonoverlapping treatments in the esophagus, duodenum, biliary tract and colon. An endoscope was used to visualize the treatment site. The treatment site was flushed with sterile saline prior to tracking the balloons in. The drug-coated balloon catheters were tracked down the working channel of the endoscope until they reached the treatment site. Prior to inflation the drug coating was allowed to hydrate for 1 minute. The drug-coated balloon catheters were then inflated to rated burst pressure at the treatment sites for 2 min to release drug and additive, then deflated and withdrawn from the pigs. The pigs were sacrificed so the tissue drug content could be measured after 1 hour and the residual drug remaining on the balloon after use was analyzed.

The pig tissue drug concentration from the esophagus, duodenum, biliary tract and colon samples was 19.5, 28.7, 309.0 and 5.5 μg/g respectively at 1 hour. The residual balloon content as a percent of the original drug loading from the samples ranged from 1.35-65.5%.

### Example IV-2. GI Preclinical Study 2

Eighty balloon catheters (10, 15 and 18 mm in diameter and 55 mm in length) were inflated to 1 atmosphere and wiped with an ethanol wipe to clean the balloon surface. The balloons were coated using Formulation 23 from Example I-1 with sufficient coating solution to achieve 3.5 microgram paclitaxel per square mm of balloon surface. The balloons were then dried, folded, sheathed, packaged in a Tyvek pouch and ethylene oxide sterilized in preparation for animal testing.

For this study male pigs were used. Pretreatment endoscopy was conducted to measure the inner diameter of the esophagus, duodenum, and colon treatment sites before drug-coated balloon treatment. The esophagus, duodenum and colon treatment site diameters were approximately 15-18 mm. The balloon catheters were chosen such that the stretch ratio for the treatments was approximately 1.1-2.2. Drug-coated balloon catheters were used with nonoverlapping treatments in the esophagus, duodenum and colon. An endoscope was used to visualize the treatment site. The treatment site was flushed with sterile saline prior to tracking the balloons in. The drug-coated balloon catheters were tracked down the working channel of the endoscope until they reached the treatment site. Prior to inflation the drug coating was allowed to hydrate for 1 minute. The drug-coated balloon catheters were then inflated to rated burst pressure at the treatment sites for 2 min to release drug and additive, then deflated and withdrawn from the pigs. The tissue drug content was measured after 1 hour. The pig tissue drug concentration from the esophagus, duodenum, and colon samples was 66.1, 40.3, and 127.0 μg/g respectively at 1 hour.

### Example IV-3. GI Preclinical Study 3

Twenty-eight balloon catheters (6 and 8 mm in diameter by 30 mm in length) were inflated to 1 atmosphere and wiped with an ethanol wipe to clean the balloon surface. The balloons were coated using Formulation 23 from Example I-1 with sufficient coating solution to achieve 3.5 microgram paclitaxel per square mm of balloon surface. The balloons were then dried, folded, sheathed, packaged in a Tyvek pouch and ethylene oxide sterilized in preparation for animal testing.

For this study male pigs were used. Endoscopic retrograde cholangiopancreatography (ERCP) with sphincterotomy of the ampula of vater was conducted to take a cholangiogram of the biliary tract and identify the treatment sites. The biliary tract treatment site diameters were 4-8 mm. The balloon catheters were chosen such that the stretch ratio for the treatments was approximately 1.5-2.2. Drug-coated balloon catheters were used with nonoverlapping treatments in the biliary tract. The drug-coated balloon catheters were tracked down the working channel of the duodenoscope until they reached the treatment site. Prior to inflation the drug coating was allowed to hydrate for 1 minute. The drug-coated balloon catheters were then inflated to rated burst pressure at the treatment sites for 2 min to release drug and additive, then deflated and withdrawn from the pigs. The tissue drug content was measured after 1 hour. The pig tissue drug concentration from the biliary tract samples was 170.0 μg/g at 1 hour.

### Example IV-4. Chronic GI Preclinical Study with Sirolimus Coated Balloon Catheters

42 balloon catheters (18 mm in diameter and 65 mm in length) were inflated to 1 atmosphere and wiped with an ethanol wipe to clean the balloon surface. The balloons were evenly coated using Formulation C6SsusF13-3, MSF25 from Example I-1 with sufficient coating solution to achieve 9.2 mg drug dosing. The DCBs were used with nonoverlapping treatments in the esophagus, duodenum and colon. An endoscope was used to visualize the treatment site. The treatment site was flushed with sterile saline prior to tracking the balloons in. The drug-coated balloon catheters were tracked down the working channel of the endoscope until they reached the treatment site. Prior to inflation the drug coating was allowed to hydrate for 1 minute. The drug-coated balloon catheters were then inflated to rated burst pressure at the treatment sites for 2 min to release drug and additive, then deflated and withdrawn from the pigs and collected for residual drug content measurements. After treatments the pigs were survived for 1 hours, 7 day, and 28 days and then the treated tissue was excised and bisected in the necropsy lab with one half of the sample being assayed for drug content and the other half sample going to histology. The measured sirolimus drug concentration in the various tissues can be seen in Table 13. The residual amount of drug left on the balloons post treatment can be seen in Table 14.

TABLE 13
Formulation C6SsusF13-3, MSF25 Sirolimus Coating
measured drug concentrations in various tissues.

| | | | Rapa- | Total | |
|---|---|---|---|---|---|
| | | Sample | mycin | Rapa- | Day |
| | | weight | Conc. | mycin | Col- |
| Subject | Segment | (g) | (ng/g) | (ng) | lected |
| 20P1311 | Esophagus Prox | 4.8272 | 6430 | 31000 | 0.04 |
| 20P1311 | Esophagus Distal | 4.1582 | 7600 | 31600 | 0.04 |
| 20P1311 | Duodenum Prox | 4.1988 | 1300 | 5460 | 0.04 |
| 20P1311 | Duodenum Distal | 4.1128 | 1040 | 4280 | 0.04 |
| 20P1311 | Colon Prox | 7.4891 | 1820 | 13600 | 0.04 |
| 20P1311 | Colon Distal | 6.7983 | 1920 | 13100 | 0.04 |
| 20P1311 | Urethra Prox | 2.3321 | 950 | 2220 | 0.04 |
| 20P1311 | Urethra Distal | 2.2199 | 23700 | 52600 | 0.04 |
| 20P1389 | Esophagus Prox | 1.7552 | 76.1 | 134 | 7 |
| 20P1389 | Esophagus Distal | 2.0545 | 37.7 | 77.5 | 7 |
| 20P1389 | Duodenum Prox | 3.1134 | 52.0 | 162 | 7 |
| 20P1389 | Duodenum Distal | 3.3873 | 63.4 | 215 | 7 |
| 20P1389 | Colon Prox | 2.8320 | 73.2 | 207 | 7 |
| 20P1389 | Colon Distal | 4.7838 | 78.8 | 377 | 7 |
| 20P1389 | Urethra Prox | 0.7627 | 46.7 | 35.6 | 7 |
| 20P1389 | Urethra Distal | 0.4232 | 25.7 | 10.9 | 7 |
| 20P1390 | Esophagus Prox | 2.2717 | 75.4 | 171 | 7 |
| 20P1390 | Esophagus Distal | 1.9296 | 56.5 | 109 | 7 |
| 20P1390 | Duodenum Prox | 3.6512 | 77.5 | 283 | 7 |
| 20P1390 | Duodenum Distal | 4.0159 | 57.4 | 231 | 7 |
| 20P1390 | Colon Prox | 2.2745 | 92.5 | 210 | 7 |
| 20P1390 | Colon Distal | 5.1932 | 107 | 556 | 7 |
| 20P1309 | Esophagus Prox | 3.5945 | 5.34 | 19.2 | 28 |
| 20P1309 | Esophagus Distal | 4.5356 | 1.96 | 8.89 | 28 |
| 20P1309 | Duodenum Prox | 2.6779 | 5.70 | 15.3 | 28 |
| 20P1309 | Duodenum Distal | 2.8140 | 4.63 | 13.0 | 28 |
| 20P1309 | Colon Prox | 3.0973 | 20.7 | 64.1 | 28 |
| 20P1309 | Colon Distal | 4.3016 | 6.79 | 29.2 | 28 |
| 20P1309 | Urethra Prox | 2.3129 | 5.88 | 13.6 | 28 |
| 20P1309 | Urethra Distal | 0.9159 | 3.52 | 3.22 | 28 |
| 20P1310 | Esophagus Prox | 3.3146 | 5.21 | 17.3 | 28 |
| 20P1310 | Esophagus Distal | 2.8290 | 1.55 | 4.38 | 28 |
| 20P1310 | Duodenum Prox | 3.6420 | 4.51 | 16.4 | 28 |
| 20P1310 | Duodenum Distal | 3.3454 | 2.31 | 7.73 | 28 |
| 20P1310 | Colon Prox | 3.0805 | 4.35 | 13.4 | 28 |
| 20P1310 | Colon Distal | 8.0090 | 6.54 | 52.4 | 28 |

TABLE 14
Residual amount of drug left on the balloons
post treatment for C6SsusF13-3, MSF25.

| Subject | Treatment Location | Residual Sirolimus (mg) | % of Dose |
|---|---|---|---|
| 20P1309 | Prox Duo | 0.5 | 5.7% |
| 20P1309 | Dist Eso | 2.3 | 25.4% |
| 20P1309 | Prox Colon | 1.2 | 13.5% |
| 20P1309 | Dist Duo | 1.1 | 11.9% |
| 20P1309 | Dist Colon | 4.5 | 49.2% |
| 20P1309 | Prox Eso | 3.4 | 36.8% |
| 20P1310 | Prox Eso | 2.6 | 27.9% |
| 20P1310 | Dist Duo | 1.5 | 16.8% |
| 20P1310 | Colon Dist | 4.2 | 45.8% |
| 20P1310 | Prox Colon | 4.1 | 44.7% |
| 20P1310 | Dist Eso | 2.5 | 26.8% |
| 20P1310 | Prox Duo | 0.8 | 9.2% |
| 20P1311 | Dist Duo | 1.0 | 10.4% |
| 20P1311 | Dist Colon | 2.2 | 23.6% |
| 20P1311 | Prox Duo | 1.6 | 17.5% |
| 20P1311 | Dist Eso | 4.5 | 48.6% |
| 20P1311 | Prox Colon | 1.2 | 13.4% |
| 20P1311 | Prox Eso | 3.6 | 39.6% |
| 20P1389 | Prox Colon | 1.9 | 20.1% |
| 20P1389 | Prox Duo | 0.5 | 5.0% |
| 20P1389 | Dist Eso | 2.0 | 21.3% |
| 20P1389 | Prox Eso | 2.6 | 28.4% |
| 20P1389 | Dist Duo | 4.5 | 49.0% |
| 20P1389 | Dist Colon | 0.2 | 2.0% |
| 20P1390 | Prox Colon | 5.7 | 62.3% |
| 20P1390 | Dist Colon | 2.5 | 27.4% |
| 20P1390 | Dist Eso | 1.7 | 18.3% |
| 20P1390 | Prox Duo | 0.4 | 4.0% |
| 20P1390 | Dist Duo | 1.3 | 14.7% |
| 20P1390 | Prox Eso | 2.0 | 21.6% |
| 21P0012 | Prox Duo | 0.4 | 4.4% |
| 21P0012 | Prox Eso | 1.6 | 17.1% |
| 21P0012 | Dist Eso | 3.7 | 40.7% |
| 21P0012 | Dist Duo | 1.3 | 14.2% |
| 21P0012 | Prox Colon | 2.2 | 24.1% |
| 21P0012 | Dist Colon | 2.9 | 32.0% |

### Part V. Human Clinical Testing Drug-Coated Balloons in the Gastrointestinal Tract.

In this Part, the drug coating on the drug-coated balloon included paclitaxel and an excipient as a homogeneous blend, with a coating dose of paclitaxel of 3.5 microgram/mm². The excipient was pentraerythritol ethoxlate (PEE) 15/4. The weight ratio of the excipient to paclitaxel in the coating was 1:3.

### Example V-1. Human Clinical Subject a with an Esophageal Stricture Treated with an Uncoated Predilation Balloon Followed by an 18 mm Nominal Diameter Drug-Coated Balloon Catheter

75-year-old male subject A had a 2.0 cm length by 9 mm diameter stricture in his esophagus. Specifically located ⅔rds the length of the esophagus from the mouth. This was determined by conducting an esophogram. The human clinical subject had a baseline dysphagia handicap index score of 42. First a gastroscope was inserted into the mouth and down the esophagus to the stricture. Next a predilation balloon that had a nominal diameter of 12 mm and a length of 55 mm was tracked into the working channel of the gastroscope and positioned so the balloon crossed the stricture. The predilation balloon was inflated to 9 atmospheres with a syringe that had a pressure gauge on it and held at pressure for 5 minutes. The syringe contained a mixture of saline and contrast media. A fluoroscopic image was acquired to ensure the balloon had a uniform expansion. Next the predilation balloon was removed and another predilation balloon that had a nominal diameter of 15 mm and a length of 55 mm was tracked into the working channel of the gastroscope and positioned so the balloon crossed the stricture. The second predilation balloon was inflated to 7 atmospheres which corresponded to a diameter of 16.5 mm and was held at pressure for 5 minutes. Next a drug-coated balloon that had a nominal diameter of 18 mm and a length of 55 mm was inserted into the gastroscope. The drug-coated balloon was positioned such that the balloon body completely covered the predilated stricture area. The drug-coated balloon was held in position for at least 1 minute prior to inflating to hydrate the coating. Then the drug-coated balloon was inflated with a mixture of saline and contrast media using the syringe that had a pressure gauge on it. The balloon was inflated to 4.5 atmospheres, achieved an inflated diameter of 19 mm, and was held at the inflation pressure for 5 minutes. Then the balloon was deflated and withdrawn from the human subject. The stretch ratio for the 18 mm nominal diameter drug-coated balloon was 2.1. The diameter of the dilated stricture was 18 mm after dilation. The human clinical subject had follow-up visits at 30, 90, and 180 days to measure esophagus diameter, dysphagia handicap index score, and body mass. The human clinical subject had an esophagus diameter increase from 9.0 mm to 20.0 (122% increase), 20.0 (122% increase), and 20.0 mm (122% increase) at follow-up visits of 30, 90, and 180 days respectively. The human clinical subject had dysphagia handicap index score improvement from 42 to 6 (86% reduction), 6 (86% reduction), and 2 (95% reduction) at follow-up visits of 30, 90, and 180 days respectively. The human clinical subject had body mass change from 65 kg to 66.5, 65, and 64.5 kg at follow-up visits of 30, 90, and 180 days respectively. Patient A did not receive any stricture reinterventions after being treated with a drug-coated balloon.

### Example V-2. Human Clinical Subject B with a Large Bowel Stricture Treated with an Uncoated Predilation Balloon Followed by an 18 mm Nominal Diameter Drug-Coated Balloon Catheter

35-year-old subject B had a 0.5 cm length by 12 mm diameter stricture in his colon. Specifically located at the colon-rectum junction. This was determined by conducting an colonoscopy. The human clinical subject had a baseline obstructive symptom score of 69 with severe constipation, inability of have a bowel movement, swelling and distention of the abdomen, and vomiting. First a colonoscope was inserted into the anus and up adjacent to the stricture. Next a predilation balloon that had a nominal diameter of 18 mm and a length of 55 mm was tracked into the working channel of the colonoscope and positioned so the balloon crossed the stricture. The predilation balloon was inflated to 6 atmospheres with a syringe that had a pressure gauge on it and held at pressure for 5 minutes. The syringe contained a mixture of saline and contrast media. A fluoroscopic image was acquired to ensure the balloon had a uniform expansion. Next the predilation balloon was removed and a drug-coated balloon that had a nominal diameter of 18 mm and a length of 55 mm was inserted into the colonoscope. The drug-coated balloon was positioned such that the balloon body completely covered the predilated stricture area. The drug-coated balloon was held in position for at least 1 minute prior to inflating to hydrate the coating. Then the drug-coated balloon was inflated with a mixture of saline and contrast media using the syringe that had a pressure gauge on it. The balloon was inflated to 6 atmospheres, achieved an inflated diameter of 20 mm, and was held at the inflation pressure for 5 minutes. Then the balloon was deflated and withdrawn from the human subject. The stretch ratio for the 18 mm nominal diameter drug-coated balloon was 1.7. The diameter of the dilated stricture was 19 mm after dilation. The human clinical subject had follow-up visits at 30, 90, and 180 days to measure obstructive symptoms. The human clinical subject had obstructive symptom score improvement from 69 to 0 (100% reduction), 0 (100% reduction), and 0 (100% reduction) at follow-up visits of 30, 90, and 180 days respectively. Patient A did not receive any stricture reinterventions after being treated with a drug-coated balloon.

### Example V-3. Kaplan-Meier Freedom from Reintervention of Esophageal and Bowel Strictures

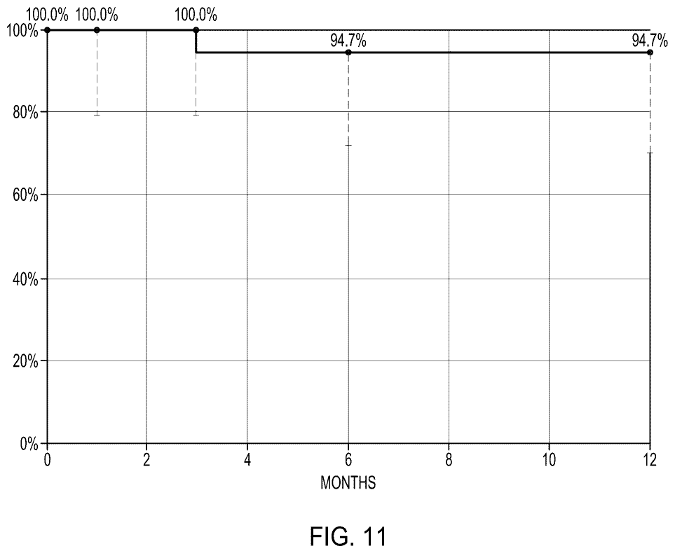

A total of 19 subjects were treated with paclitaxel coated balloons in their esophagus and bowel and only one patient required retreatment through 12 months follow-up. FIG. 11 shows a survival curve analysis (i.e., freedom from reintervention Kaplan-Meier curve), and shows an estimate for freedom from reintervention at 12 months to be 94.7%.

### Example V-4. Human Clinical Subject C with a Biliary Tract Stricture Treated with an Uncoated Predilation Balloon Followed by an 8 mm Nominal Diameter Drug-Coated Balloon Catheter

A 68-year-old male subject C had a 1.0 cm length by 4 mm diameter stricture in his biliary tract. Specifically located between the common bile duct and common hepatic duct. This was determined by conducting ERCP. The human clinical subject had an indwelling biliary drainage tube prior to being treated. First a duodenoscope was inserted into the mouth and positioned near the ampulla of vater. Next a snare was used to remove the indwelling drainage tube. Next a sphincterotome was used to cannulate the biliary duct and advance a guidewire. Next a predilation balloon that had a nominal diameter of 6 mm and a length of 40 mm was tracked into the working channel of the duodenoscope and positioned so the balloon crossed the stricture. The predilation balloon was inflated to 11 atmospheres with a syringe that had a pressure gauge on it and held at pressure for 3 minutes. The syringe contained a mixture of saline and contrast media. A fluoroscopic image was acquired to ensure the balloon had a uniform expansion. The residual stenosis was originally 70% and was reduced to 40% post predilation. Next the predilation balloon was removed and a drug-coated balloon that had a nominal diameter of 8 mm and a length of 50 mm was inserted into the duodenoscope. The drug-coated balloon was positioned such that the balloon body completely covered the predilated stricture area. The drug-coated balloon was held in position for at least 1 minute prior to inflating to hydrate the coating. Then the drug-coated balloon was inflated with a mixture of saline and contrast media using the syringe that had a pressure gauge on it. The balloon was inflated to 6 atmospheres, achieved an inflated diameter of 8.8 mm, and was held at the inflation pressure for 5 minutes. The residual stenosis was originally 40% and was reduced to 12% post DCB treatment. Then the balloon was deflated and withdrawn from the human subject. The stretch ratio for the 8 mm nominal diameter drug-coated balloon was 2.2. The diameter of the dilated stricture was 8 mm after dilation. The human clinical subject had follow-up visits at 180 days to measure stricture diameter. The human clinical subject had biliary duct diameter improvement from 4 to 9 mm at 180 days. Patient C did not receive any stricture reinterventions after being treated with a drug-coated balloon.

### Part VI.

### Test Methods

In-vitro tissue adhesion testing. To evaluate if drug-coated balloon (DCB) coatings transfer drug to arterial tissue within 30 seconds of inflation and deflation and to test if the transferred drug is retained in the tissue, an in-vitro (bench) tissue testing experiment was undertaken. In this test a porcine vascular tissue section with a larger diameter than the DCB being tested was used. The arterial tissue was cut in length such that it is bigger than the DCB balloon length being tested. Phosphate buffered saline (PBS) solution at 37° C. (+/−1° C.) was used as the media to simulate blood. In a typical test procedure, vacuum was applied to DCB, the balloon protector sheath was removed, balloon was dipped in 37° C. PBS for 30 secs, folded balloon was inserted into the arterial tissue sample, a small weight was applied on the tissue to maintain contact with the folded balloon, DCB was inflated to nominal pressure and was held there for 30 seconds, DCB was deflated and held for 30 seconds, weight on the balloon was removed, and the DCB is removed from the artery. The artery sample was then tested either with dynamic or static adhesion methods. In the dynamic adhesion testing, the artery sample was exposed to 37° C. PBS recirculating flow at 70 mL/min using a peristaltic pump for 1 hr and the drug content post 1 hr flow was tested with HPLC assay test method. In static adhesion testing arterial tissue sections post drug transfer were immersed in excess PBS in an APP2 chamber with 100 rpm rotation at 37° C. for overnight and the drug content was tested. It was learned that any drug leaching out of the microspheres would degrade quickly in 37° C. PBS. Therefore, if any drug content was measured in the tissue after either static or dynamic adhesion testing, it would mean that not only did the drug laden microspheres were transferred to the tissue but also that the drug did not leach out of the microspheres or that the microspheres themselves did not dislodge from the tissue surface. Static adhesion testing overnight gave similar results as 1 hr dynamic adhesion testing and to expedite the testing process, most of the data was collected in dynamic adhesion mode. Residual drug in the tissue as a % of label claim was used to assess drug transfer to arterial tissue. The label claim was the total amount of drug on the balloon catheter; for example, a 3×20 balloon with 1.8 μg/mm² drug density in the coating has a label claim of π*3*20*1.8=339 μg drug. Balloon dimensions are given in mm unless otherwise indicated.

In-vitro tracking testing. To evaluate if DCB coatings can retain sufficient coating while being advanced into the tortuous arterial anatomy, in-vitro testing was undertaken with a mockup vessel flow model including a guide catheter and silicone tubing. A Y valve was used at the entry port of the 6Fr guide catheter with one arm going into a Tuohy valve and the arm into tubing that goes into the peristaltic pump. A Tuohy valve at the guide catheter entrance permits the introduction of DCB into the flow model. At the tip of silicone tubing another Y valve was used with one arm ending closed Tuohy valve and the other arm going into tubing that goes to the peristaltic pump. This way 37° C. water was constantly recirculated through the guide catheter+silicone tubing model during the track testing with the peristatic pump. The silicone tube that has a curvature to mimic the tortuosity of human coronary arterial anatomy. 37° C. water was continually pumped through the model at 35 mL/min. In a typical test procedure, vacuum was applied to a DCB, its balloon protector sheath was removed, a Tuohy valve was opened to insert the DCB into the guide catheter. The DCB was advanced all the way through the curved silicone tubing. At the end of the silicone tubing another Tuohy valve was opened, to advance the DCB out of the flow model, and the balloon section was cut to test residual drug retained on the DCB per HPLC test method. Residual drug as a % of label claim was used to assess drug tracking losses.

Zeta potential testing. It is well known in the literature that a positive or cationic charged coating surface can adhere better to inherently anionic arterial tissue surfaces. Zeta potential can capture the ionic charge present in the coatings used on DCB. To test the zeta potential of drug coated balloon formulations, a Beckman Coulter Desla Nano C analyzer was used. In this testing, a DCB was immersed in 20 mL clean water and was inflated and held on a sonicator for 5 min to detach the coating from the balloon and to disperse it in water. Then a 1-2 mL sample of the water with the DCB suspension was injected into the analyzer, to determine Zeta potential in mV.

In-vivo porcine coronary anatomy model. To test various aspects of how a DCB performs in vivo, porcine coronary animal testing was undertaken. In this study, a 6Fr guide catheter along with a guide wire was inserted into the aorta of a heparinized pig through carotid or femoral access. Coronary artery anatomy was imaged under fluoroscopy to identify three arterial segments including left anterior descent LAD, right coronary artery RCA and the circumferential artery CX to identify regions with suitable side branch landmarks and 2.5-3.5 mm diameter with lengths 15-30 mm. A denudation POBA catheter was used to confirm the angiographic diameter assessments and to denude the treated arterial sites. DCB samples from various examples were advanced through the guide catheter under fluoroscopy to enter the three coronary artery branches. Once reaching the desired pre-chosen landmarks, the DCB was inflated to achieve 10% over expansion and held for 30-60 seconds under inflation. All DCB were inflated to below rated burst pressure. Contrast was injected to confirm the inflated balloon achieved full apposition and the blood flow in the artery was stopped and that there is no leakage of contrast past the balloon. After the desired inflation time, the balloons were deflated and removed from the animal. Care was taken to not scrape off any coating either during introduction or exiting the Touhy valve on the guide catheter. Post treatment, the animals were sutured up and returned to the animal lab facility for monitoring for the duration of study that ranged from 1 day to 1 month. At explant time, femoral or carotid access was used to advance a guide wire and a guide catheter to the treated site. Contrast was injected to measure the diameter of the treated site. In some studies baseline and post treatment EKG measurements were also taken. For tissue pk measurements the animal was necropsied and flowing the print outs of angiographic measurements, arterial segments from treated sites were harvested and sent to drug quantification testing.

Microsphere production. Drug laden PLGA polymeric microspheres were produced using oil in water suspension methods well known in the literature. Briefly, drug, PLGA polymer and microparticle surface modifying phospholipid compound were dissolved in an oil phase, typically dichloromethane solvent. This oil phase was dispersed in a water phase containing polyvinyl alcohol suspension stabilizer using high-pressure microfluidic apparatus. The oil water mixture was then mixed with an excess amount of water for an hour under a fume hood, to further stabilize the oil suspension particles, evaporate dichloromethane and lower the concentration of polyvinyl alcohol. Particle size measurements were made after the 1-hour water mix step and then the whole batch was centrifuged to separate the microspheres from the liquid phase. The centrifuged cake was redispersed in excess of fresh water using an ultrasonic mixing horn and then centrifuged again for a second time to further reduce the concentration of polyvinyl alcohol. The centrifuged cake was then dispersed again in minimal amount of water with an ultrasonic mixing horn and transferred into a tray which was then placed in a lyophilization oven for drying. Lyophilized microsphere powder was tested for drug content using HPLC test methods. Particle size and size distribution were measured with laser scattering analyzers and the microsphere formulation and process was optimized to produce particle sizes anywhere between 1 to 5 μm mean diameter (D50) with narrow particle size distributions.

All drug concentrations listed in the Examples are weight percentages. The microspheres had the concentration of drug described in each Example. The microparticle surface modifying phospholipid compound used was of 1,2-disteroyl-sn-glycero-3-phosphatidylcholine (DSPC) for each Example, used at a concentration of 1 wt %. The concentration of PLGA used in each Example formed the remainder of the microspheres, and was 100 wt %-1 wt %-drug wt %; for example, for 29 wt % drug, the concentration of drug was 29 wt %, the concentration of DSPC was 1 wt %, and the concentration of PLGA was 100 wt %-1 wt %-29 wt %=70 wt %. The pentaerythritol ethoxylate (PEE) used in the Examples was 15/4 EO/OH with an average molecular weight of 797.

### Example VI-1. Lot 2LYL-21 B1

A microsphere batch (MSF) with mean diameter (D50) 2.77 μm with Sirolimus drug content of 26.3% was dispersed in water using an ultrasonic mixer. This coating suspension was measured for drug content and then applied as coating on balloon catheter with diameter 3 mm and 30 mm length to achieve a target dose density of 1.8 μg/mm²+/−10%. The DCB coating was dried and then pleated and folded (P/F) into a balloon protector sheath (BPS) and packaged in a foil pack. This lot of DCB was then tested for zeta potential using the methods described above. A Zeta potential value of −32.09 mV was measured.

### Example VI-2. Lot 2LYL-21 B2T1

Similar coating suspension, balloon coating, P/F and final packaging were conducted as the one described above but a polylysine polymer was added to the water at 2.6% of the weight of MSF during the ultrasonic mixing. Sirolimus drug content in the coating was maintained at 1.8 μg/mm²+/−10%. Additionally, a cyclohexane-based topcoat coating of POPC and polylysine at an 8:2 weight ratio was also applied at 10% of the weight of MSF. This lot of DCB was tested for Zeta potential using the methods described above. The Zeta potential value was 49.67.

### Example VI-3. Lot 2LYL-21 B3T5

Similar coating suspension, balloon coating, P/F and final packaging were conducted as the one described above but a polylysine polymer was added to the water at 10% of the weight of MSF during the ultrasonic mixing. Sirolimus drug content in the coating was maintained at 1.8 μg/mm²+/−10%. Additionally, a cyclohexane-based topcoat coating of POPC and BHT at a 1:1 weight ratio was also applied at 16% of the weight of MSF. This lot of DCB was tested for Zeta potential using the methods described above. A Zeta potential value of 68.53 mV was measured.

Taken together, Examples 1, 2, and 3 show that the use of a cationic polymer significantly increases the zeta potential of MSF-containing DCB coatings from −32.09 mV to >49 mV.

### Example VI-4. Lot 2LYL22 B1T1

To a similar coating as in Example 2 but with 1% polylysine in the base coating, a cyclohexane-based topcoat was applied including POPC & BHT at 1:1 and applied at 12.2% of the weight of MSF on a 5×40 mm balloon catheter. Sirolimus drug content in the coating was maintained at 1.8 μg/mm²+/−10%. This coated catheter was P/F and packaged and tested for ex-vivo tissue adhesion in the static test mode as explained above in the test methods. Residual drug measured in the tissue segment in two replicates was measured at 33% and 30.8%, respectively.

### Example VI-5. Lot 2LYL22 B1T2

To a similar coating as in Example 2 but with 1% polylysine in the base coating, a cyclohexane-based topcoat was applied including POPC & BHT at 1:1 and applied at 18.4% of the weight of MSF on a 5×40 balloon catheter. Sirolimus drug content in the coating was maintained at 1.8 μg/mm²+/−10%. This coated catheter was P/F and packaged and tested for ex-vivo tissue adhesion in the static test as explained above in the test methods. Residual drug measured in the tissue segment in two replicates was measured at 27.5% and 26.3%, respectively.

### Example VI-6. Lot 2LYL22 B1T3

To a similar coating as in Example 2 but with 1% polylysine in the base coating, a cyclohexane-based topcoat was applied including POPC & BHT at 1:1 and applied at 24.5% of the weight of MSF on a 5×40 balloon catheter. Sirolimus drug content in the coating was maintained at 1.8 μg/mm²+/−10%. This coated catheter was P/F and packaged and tested for ex-vivo tissue adhesion in the static test mode as explained above in the test methods. Residual drug measured in the tissue segment in two replicates was measured at 29.3% and 23.3%, respectively.

Taken together, Examples 4, 5 & 6 show that sufficient drug is transferred and retained at the tissue surface after an overnight agitation in 37° C. PBS. From Examples 1 through 6, it is evident that using polylysine polymer additions can boost tissue adhesion and retention in the arterial tissue and could be a result of increased zeta potential of these coatings.

### Example VI-7. Lot 2LYL23 B1

A coating similar to Example 1 was deposited on a 5×40 mm catheter at 1.8 μg/mm² dose density +/−10% and was P/F and packaged and was used for ex-vivo static adhesion testing as explained above in the test methods. Residual drug measured in the tissue segment in two replicates was measured at 10.9% and 10.4% of label claim, respectively.

### Example VI-8. Lot 2LYL23 B1T1

To the coating in Example 7 on a 5×40 balloon catheter a cyclohexane-based topcoat was added containing POPC & BHT at 1:1 and was dosed at 10% by weight of MSF. These catheters were P/F and packaged and were used for ex-vivo static adhesion testing as explained above in the test methods. Residual drug measured in the tissue segment in two replicates was measured at 18.9% and 17.0% of label claim, respectively.

### Example VI-9. Lot 2LYL23 B2

A coating similar to Example 2 was applied on a 5×40 catheter and Sirolimus drug content was maintained at 1.8 μg/mm²+/−10%. These catheters were P/F and packaged and were used for ex-vivo static adhesion testing as explained above in the test methods. Residual drug measured in the tissue segment in two replicates was measured at 39.1% and 27.4% of label claim, respectively.

Taken together, Examples 7, 8 & 9 show that while POPC/BHT topcoat can boost tissue adhesion from 10% to 17%, use of polylysine produces coatings that yield higher drug levels in the tissue at >27%.

### Example VI-10. Lot PLH-01

A microsphere batch with D50 2.58 μm and Sirolimus drug content at 27.9% was mixed with water in an ultrasonic mixer and then polylysine polymer was added to the coating suspension at 10% by weight of MSF and mixed again in the ultrasonic mixer. The coating suspension was applied on 5×40 mm balloon catheters targeting Sirolimus drug content of 1.8 μg/mm²+/−10%. To this coating a water solution of hyaluronic acid (HA) was applied as a second layer such that polylysine to HA was maintained at 2:1 ratio by weight. The coated catheters were P/F and packaged in a foil pack and were used for tissue adhesion testing per the static and dynamic adhesion methodologies described above. Residual drug measured in the tissue segment per the static adhesion test method was 33.1% and per the dynamic adhesion test method was 38.8% of label claim.

### Example VI-11. Lot 2LYL-26

A microsphere batch with D50 2.72 μm and Sirolimus drug content at 27.9% was mixed with water in an ultrasonic mixer and then polylysine polymer was added to the coating suspension at 1% by weight of MSF and mixed again in the ultrasonic mixer. The coating suspension was applied on 3×20 mm and 5×40 mm balloon catheters targeting Sirolimus drug content of 1.8 μg/mm²+/−10%. To this coating a cyclohexane-based coating was applied including POPC & BHT at 1:1 wt ratio and at 10% each by weight of MSF. The coated catheters were P/F and packaged in a foil pack and were used for tissue adhesion testing per the static and dynamic adhesion methodologies described above. Residual drug measured in the tissue segment per the static adhesion test method was 13.0% and per the dynamic adhesion test method was 17.6% of label claim.

### Example VI-12. Lot 2LYL-27

A microsphere batch with D50 2.72 μm and Sirolimus drug content at 27.9% was mixed with water in an ultrasonic mixer and then polylysine polymer was added to the coating suspension at 2.5% by weight of MSF and mixed again in the ultrasonic mixer. The coating suspension was applied on 3×20 mm and 5×40 mm balloon catheters targeting Sirolimus drug content of 1.8 μg/mm²+/−10%. To this coating a cyclohexane based coating was applied including POPC & BHT at 1:1 wt ratio and at 5% each by weight of MSF. The coated catheters were P/F and packaged in a foil pack and were used for tissue adhesion testing per the static and dynamic adhesion methodologies described above. Residual drug measured in the tissue segment per the static adhesion test method was 26.6% and per the dynamic adhesion test method was 26.03% of label claim.

Taken together, Examples 10, 11, and 12 show that static and dynamic mode of adhesion test methods produce similar results (within 5%) and that use of polylysine can boost tissue adhesion and retention of sirolimus drug encapsulated within the microspheres.

### Example VI-13. Lot PLH-10

A microsphere batch with D50 2.55 μm and Sirolimus drug content at 27.6% was mixed with water in an ultrasonic mixer and then polylysine polymer was added to the coating suspension at 5% by weight of MSF and mixed again in the ultrasonic mixer. Then HA water solution was added to the coating suspension such that polylysine to HA was maintained at 3.33:1 weight ratio and was again mixed on the ultrasonic mixer. The coating suspension was applied on 3×20 mm and 5×40 mm balloon catheters targeting Sirolimus drug content of 1.8 μg/mm²+/−10%. To this coating a cyclohexane-based coating solution including POPC and BHT at 1:1 wight ratio was applied such that POPC measured at 10% and 20% by weight of MSF. The coated catheters were P/F and packaged in a foil pack and were used for coating durability per in-vitro tracking test methodologies described above. Residual drug measured in the post tracking was measured at 13.4% for 10% POPC/BHT and 33.2% for 20% POPC/BHT.

### Example VI-14. Lot PLH-12

A microsphere batch with D50 3.21 μm and Sirolimus drug content at 26.8% was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 3.33:1 weight ratio and was again mixed on the ultrasonic mixer. The coating suspension was applied on 3×20 mm and 5×40 mm balloon catheters targeting Sirolimus drug content of 1.8 μg/mm²+/−10%. The same coating was repeated with one change, polyarginine replacing polylysine. To these coatings a cyclohexane-based coating solution including POPC and BHT at 1:1 wight ratio was applied such that POPC measured at 10% and 20% by weight of MSF. The coated catheters were P/F and packaged in a foil pack and were used for in-vitro tracking test methodologies described above. Residual drug measured in the post tracking was measured at 10.8% for polylysine and 26.6% for polyarginine.

### Example VI-15. Lot PLH-15

A microsphere batch with D50 3.26 μm and Sirolimus drug content at 27.5% was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 5:1 weight ratio and was again mixed on the ultrasonic mixer. The coating suspension was applied on 3×20 mm and 5×40 mm balloon catheters targeting Sirolimus drug content of 1.8 μg/mm²+/−10%. To these coatings a cyclohexane-based coating solution including POPC and BHT at 1:1 wight ratio was applied such that POPC measured at 10% by weight of MSF. The coated catheters were P/F and packaged in a foil pack and were used for dynamic tissue adhesion testing and in-vitro tracking test methodologies described above. Residual drug measured in the tissue post dynamic adhesion testing was at 20.2% of label claim and the residual drug left on the balloon post tracking was measured at 19.15%.

### Example VI-16. Lot PLH-16

A microsphere batch with D50 3.26 μm and Sirolimus drug content at 27.5% was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 5:1 weight ratio and was again mixed on the ultrasonic mixer. The coating suspension was applied on 3×20 mm and 5×40 mm balloon catheters targeting Sirolimus drug content of 1.8 μg/mm²+/−10%. To these coatings three different cyclohexane-based coating solutions were applied. The first coating solution contained stearic acid 50 (a blend of stearic and palmitic acid, referred to as “SA”) measured at 10% by weight of MSF, the second coating solution contained SA and POPC at 1:1 weight ratio and the third coating solution contained POPC & DLPC at 1:1 weight ratio. The coated catheters were P/F and packaged in a foil pack and were used for dynamic tissue adhesion testing and in-vitro tracking test methodologies described above. Residual drug measured as % label claim in the tissue post dynamic adhesion testing was at 59.6% for SA, 33.3% for SA+POPC and 30.6% for POPC+DLPC. Residual drug left on the balloon as % label claim post tracking was measured at 51.29% for SA, 23.23% for SA+POPC and 5.6% for POPC+DLPC.

### Example VI-17. Lot PLH-18

A similar batch of microspheres as in Example 16 was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 5:1 weight ratio and was again mixed on the ultrasonic mixer. The coating suspension was applied on 3×20 mm and 5×40 mm balloon catheters targeting Sirolimus drug content of 1.8 μg/mm²+/−10%. To these coatings a cyclohexane-based coating solution containing SA at 10% by weight of MSF, was applied. The coated catheters were P/F and packaged in a foil pack and were used for dynamic tissue adhesion testing as described above. Residual drug measured as % label claim in the tissue post dynamic adhesion testing was at 46%.

### Example VI-18. Lot PLH-19

A similar batch of microspheres as in Example 16 was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 6.67:1 weight ratio and was again mixed on the ultrasonic mixer. The coating suspension was coated on 3×20 mm and 5×40 mm balloon targeting 1.8 μg/mm² Sirolimus drug content+/−10%. To these coatings four cyclohexane-based coatings were applied. These coating solutions contain SA at 10% by weight of MSF; SA+POPC at 2:1 weight ratio with SA at 10% by weight of MSF; SA+POPC at 3:1 weight ratio with SA at 15% by weight of MSF; and SA+POPC+BHT at 2:1:2 weight ratio with SA at 10% by weight of MSF. The coated catheters were P/F and packaged in a foil pack and were used for dynamic tissue adhesion testing and in-vitro tracking test methodologies described above. Residual drug measured as % label claim in the tissue post dynamic adhesion testing was at 50.2% for SA, 29.2% for SA+POPC 2:1, 47.6% for SA+POPC 3:1 and 23% for SA+POPC+BHT. Residual drug measured as % label claim post in-vitro tracking test was at 33.41% for SA, 36.23% for SA+POPC 2:1, 47.7% for SA+POPC 3:1 and 44.5% for SA+POPC+BHT

### Example VI-19. Lot PLH-20

A similar batch of microspheres as in Example 16 was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 6.67:1 weight ratio and was again mixed on the ultrasonic mixer. The coating suspension was applied on 3×20 mm and 5×40 mm balloon catheters targeting Sirolimus drug content of 1.8 μg/mm²+/−10%. To these coatings two cyclohexane-based coatings were applied. These coatings contain 2:1 SA:POPC with SA @6.7% of MSF or SA at 10% by weight of MSF. The coated catheters were P/F and packaged in a foil pack and were used for dynamic tissue adhesion testing and in-vitro tracking test methodologies described above. Residual drug measured as % label claim in the tissue post dynamic adhesion testing was at 44.4% for SA+POPC and at 66.5% for SA. Residual drug measured as % label claim post in-vitro tracking test was at 37.58% for SA+POPC and 38.86% for SA. Another lot of Example 19 with SA+POPC were subjected to electron beam radiation sterilization at 25 kGY dose and post sterile DCB was tested for in-vitro tracking and residual drug as % of label claim post tracking was measured at 57.5%. Yet another lot of Example 19 built at 3.0 μg/mm² sirolimus dose density was also e-beam sterilized at 25 kGY and residual drug as % of label claim post tracking was measured at 46%.

### Example VI-20. Lot PLH-21

A similar batch of microspheres as in Example 16 was mixed with water in an ultrasonic mixer and then polylysine polymer was added to the coating suspension at 10% by weight of MSF and mixed again in the ultrasonic mixer. Then HA water solution was added to the coating suspension such that polylysine to HA was maintained at 20:1 weight ratio and was again mixed on the ultrasonic mixer. The coating suspension was applied on 3×20 mm and 5×40 mm balloon catheters targeting Sirolimus drug content of 1.8 μg/mm²+/−10%. To these coatings three cyclohexane-based coatings were applied. These coatings contain SA at 10% by weight of MSF; SA:POPC at 2:1 ratio with SA @10% by weight of MSF; SA:POPC:BHT at 2:1:1 with SA at 10% by weight of MSF. The coated catheters were P/F and packaged in a foil pack and were used for dynamic tissue adhesion testing and in-vitro tracking test methodologies described above. Residual drug measured as % label claim in the tissue post dynamic adhesion testing was 42.9% for SA, 41.2% for SA+POPC and 36.1% for SA+POPC+BHT. Residual drug measured as % label claim post in-vitro tracking test was at 38.86% and 32.79% for the two replicates containing SA; 37.58% and 51.44% for the two replicated containing POPC+SA; and 47.68% for SA+POPC+BHT.

### Example VI-21

The DCB balloon sample from the example 20 coatings containing SA:POPC 2:1 with SA @6.7% by weight of MSF was used in a porcine heart to measure residual drug post implantation in an animal model. A 6Fr guide catheter inserted into the aorta and the balloon catheter was advanced through the guide catheter into the coronary artery (LAD). Once the balloon catheter reached a predetermined spot in the coronary artery, the balloon was pushed out of the vessel via an incision in the artery. The balloon stump was cut off and was tested for residual drug content. This test mimics the implant procedure of advancing the DCB all the way into the required location of inflation and measuring the residual drug content gives an approximation of drug transmission loss during the advancement stage. Residual drug content as a % of label claim left on the DCB tracked through porcine heart was measured at 49.35%. This value compares well with the average in-vitro tracking residual data, for Example 20 measured at 44.5%.

### Example VI-22. Lot PLH-22

A similar batch of microspheres as in Example 16 was mixed with water in an ultrasonic mixer and then polylysine polymer was added to the coating suspension at 10% by weight of MSF and mixed again in the ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC 2:1 with SA at 6.7% of MSF by weight, and with SA+POPC+BHT 2:1:1 with SA at 6.7% of MSF by weight. The coated catheters were P/F and packaged in a foil pack and were used for dynamic tissue adhesion testing, in-vitro tracking test methodologies described above and for porcine artery tracking loss per method described in Example 21. Residual drug measured as % label claim in the tissue post dynamic adhesion testing was 33.7% for SA+POPC and 37.2% for SA+POPC+BHT. Residual drug measured as % label claim post in-vitro track testing was 16.33% for SA+POPC and 18.1% for SA+POPC+BHT. Residual drug measured as % label claim left on DCB post tracking through porcine heart was 26.1% for SA+POPC and 13.1% for SA+POPC+BHT.

### Example VI-23. Lot PLH-23

A similar batch of microspheres as in Example 16 was mixed with water in an ultrasonic mixer and then polylysine polymer was added to the coating suspension at 10% by weight of MSF and mixed again in the ultrasonic mixer. Then HA polymer solution in water was such that polylysine to HA was at 20:1 ratio on weight basis, and the suspension was further mixed on the ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 2:1:1 ratio on weight basis and with SA at 6.7% by weight of MSF. The coated catheters were P/F and packaged in a foil pack and were used for dynamic tissue adhesion testing, in-vitro tracking test methodologies as described above. Residual drug measured as % label claim in the tissue post dynamic adhesion testing was 35% and residual drug content post in-vitro tracking was measured at 31.3%.

### Example VI-24. Lot PLH-24

A similar batch of microspheres as in Example 16 was mixed with water in an ultrasonic mixer and then polylysine polymer was added to the coating suspension at 10% by weight of MSF and mixed again in the ultrasonic mixer. Then HA polymer solution in water was such that polylysine to HA was at 6.67:1 ratio on weight basis, and the suspension was further mixed on the ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 6:3:1 ratio on weight basis and with SA at 6% by weight of MSF. The coated catheters were P/F and packaged in a foil pack and were used for dynamic tissue adhesion testing, in-vitro tracking test methodologies as described above. Residual drug measured as % label claim in the tissue post dynamic adhesion testing was 53.8% and residual drug content post in-vitro tracking was measured at 35%.

### Example VI-25. Lot PLH-25

A similar batch of microspheres as in Example 16 was mixed with water in an ultrasonic mixer and then polylysine polymer was added to the coating suspension at 6.25% by weight of MSF and mixed again in the ultrasonic mixer. Then HA polymer solution in water was such that polylysine to HA was at 8.3:1 ratio on weight basis, and the suspension was further mixed on the ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 4:2:1 ratio on weight basis and with SA at 4% by weight of MSF. The coated catheters were P/F and packaged in a foil pack and were used for dynamic tissue adhesion testing, in-vitro tracking test methodologies as described above. Residual drug measured as % label claim in the tissue post dynamic adhesion testing was 45.8% and residual drug content post in-vitro tracking was measured at 37.6%. Another 3×20 balloon of this example was tested per the porcine coronary track model detailed in Example 21. Post tracking, residual drug content measured as % label claim was measured as 47.9%.

### Example VI-26. Lot PLH-26

A similar batch of microspheres as in Example 16 was mixed with water in an ultrasonic mixer and then polylysine polymer was added to the coating suspension at 7% by weight of MSF and mixed again in the ultrasonic mixer. Then HA polymer solution in water was such that polylysine to HA was at 9.3:1 ratio on weight basis, and the suspension was further mixed on the ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC at 6:1 ratio on wt. basis and with SA at 6% by weight of MSF. The coated catheters were P/F and packaged in a foil pack and were used for dynamic tissue adhesion testing, in-vitro tracking test methodologies as described above. Residual drug measured as % label claim in the tissue post dynamic adhesion testing was 41.05% and residual drug content post in-vitro tracking was measured at 28.7%.

### Example VI-27. Lot PLH-27

A similar batch of microspheres as in Example 16 was mixed with water in an ultrasonic mixer and then polylysine polymer was added to the coating suspension at 10% by weight of MSF and mixed again in the ultrasonic mixer. Then HA polymer solution in water was such that polylysine to HA was at 6.7:1 ratio on a weight basis, and the suspension was further mixed on the ultrasonic mixer. This coating suspension was coated on 3×20 mm and 5×40 mm balloon catheters targeting Sirolimus drug content of 1.8 μg/mm²+/−10%. These catheters were then coated with two cyclohexane-based coating solutions; the first one containing SA+POPC at 9:1 ratio on a weight basis and with SA at 9% by weight of MSF and the second one containing SA+POPC+BHT at 9:1:2.5 ratio on a weight basis with SA at 9% by weight of MSF. The coated catheters were P/F and packaged in a foil pack and were used for dynamic tissue adhesion testing, in-vitro tracking test methodologies as described above. Residual drug measured as % label claim in the tissue post dynamic adhesion testing was 50.9% for SA+POPC and 70% for SA+POPC+BHT. Residual drug content post in-vitro tracking was measured at 39.16% for SA+POPC and 31.5% for SA+POPC+BHT.

### Example VI-28. Lot PLH-28

A batch of microspheres with mean diameter (D50) of 3.12 μm and Sirolimus drug content at 26.9% was mixed with water in an ultrasonic mixer with polylysine added at 10% by weight of MSF. Then a HA polymer solution in water was added such that polylysine to HA ratio was at 20:1 on a weight basis, and the coating suspension was further mixed on ultrasonic mixer. This coating suspension was coated on 3×20 mm balloon catheters targeting Sirolimus drug content of 1.8 μg/mm²+/−10%. These catheters were then coated with cyclohexane-based coating solutions containing SA+POPC+BHT at 6.7:3.3:1.5 ratio on a weight basis with SA at 6.7% by weight of MSF. The coated catheters were P/F and packaged in a foil pack and were subjected to electron beam sterilization radiation at 25 kGY+/−1 kGY dose. Post sterilized DCB were used for in-vitro tracking test methodologies as described above. Residual drug content post in-vitro tracking was measured at 51%.

### Example VI-29. Lot PLH-29

A batch of microspheres with mean diameter (D50) of 3.12 μm and Sirolimus drug content at 26.9% was mixed with water in an ultrasonic mixer with polylysine added at 7% by weight of MSF. Then a HA polymer solution in water was added such that polylysine to HA ratio was at 9.33:1 on a weight basis, and the coating suspension was further mixed on ultrasonic mixer. This coating suspension was coated on 3×20 mm balloon catheters targeting Sirolimus drug content of 1.8 μg/mm²+/−10%. These catheters were then coated with cyclohexane-based coating solutions containing SA+POPC+BHT at 4.7:2.3:1.5 ratio on a weight basis with SA at 4.7% by weight of MSF. The coated catheters were P/F and packaged in a foil pack and were subjected to electron beam sterilization radiation at 25 kGY+/−1 kGY dose. Post sterilized DCB were used for in-vitro tracking test methodologies as described above. Residual drug content post in-vitro tracking was measured at 53%.

### Example VI-30. In-Vivo Residual Drug & Pk

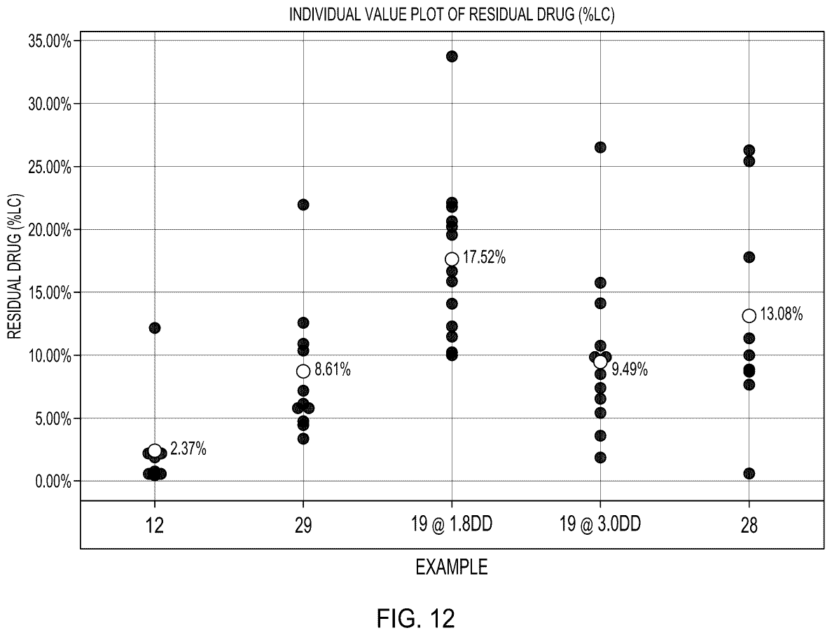

Post EB sterilized DCB from various examples and sirolimus dose densities were chosen for in-vivo drug residual testing. The DCB lots tested were Examples: 12 (@3.0 μg/mm² dose density), 19 (@1.8 and 3.0 μg/mm²), 28 (@1.8 μg/mm²), 29 (@1.8 μg/mm²) were used in a porcine coronary anatomy model. The animal testing procedure was described in the test method section. Post treatment of coronary arteries the residual drug left behind in the various DCB formulations was tested as a measure of % label claim and is plotted below. FIG. 12 illustrates drug residuals for Examples 12, 29, 19, and 28. It can be seen residual drug left on the DCB is <35% in all the formulations tested.

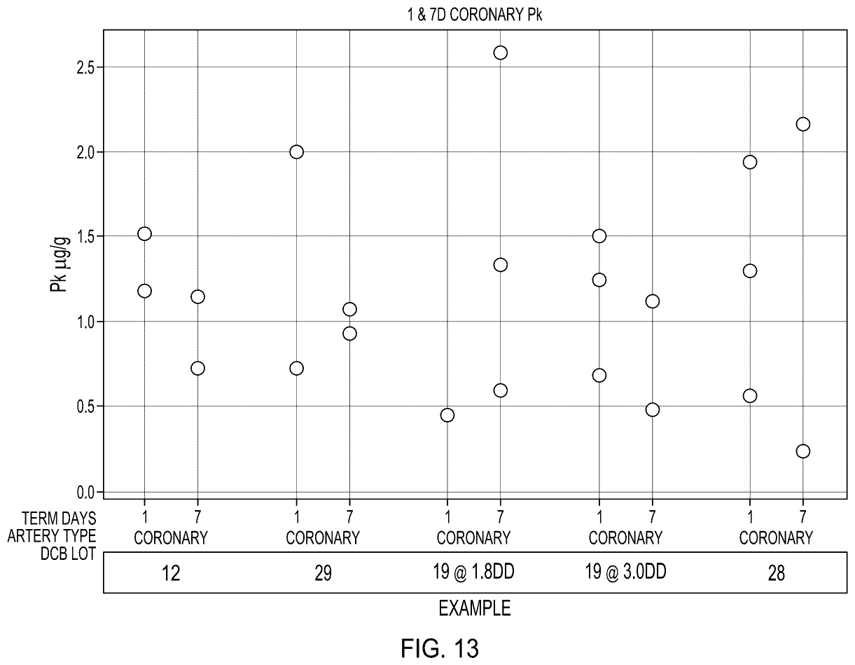

These formulations were also used to measure in-vivo pk in coronary arteries in a coronary porcine model as described in the test method section. The 1 & 7 day pk measured in μg/g are plotted in FIG. 13. Compared to a therapeutic dose level of 0.01 μg/g, substantial drug was measured in the coronary arterial tissue at 1 and 7 days. Table 15 gives the pk values for Examples 12, 19, 28, and 29 at 1 day, 7 days, and 28 days, compared to Selution (Spaulding, Christian, et al. “Comparing a strategy of sirolimus-eluting balloon treatment to drug-eluting stent implantation in de novo coronary lesions in all-comers: Design and rationale of the SELUTION DeNovo Trial.” American Heart Journal 2023, 258, 77-84) and Magictouch (Finn, Aloke, “Go Geyond Metal: Rationale behind SELUTION SLR drug-eluting balloon technology with sustained limus release”, 2025, EuroPCR, from www.PCRonline.com).

TABLE 15
Pk values for Examples 12, 19, 28, and 29.

| Lot | DCB Device | 1 day μg/g | 1 week μg/g | 1 month μg/g |
|---|---|---|---|---|
| | Selution | 2 | 0.5 | 0.4 |
| | Magictouch | 1.45 | | 0.047 |
| 2LYL-27 | Example 12 | 1.35 | 0.941 | 1.6875 |
| PLH20-2 | Example 19 1.8 DD | 0.447 | 1.502 | 1.055 |
| PLH20-3 | Example 19 3.0 DD | 1.14 | 0.8 | 0.98 |
| PLH-28 | Example 28 | 1.267 | 1.198 | 1.437 |
| PLH-29 | Example 29 | 1.363 | 1.005 | 0.649 |

### Example VI-31. Lot PLH:30

A batch of microspheres with mean diameter (D50) of 2.79 μm and Sirolimus drug content at 29% was mixed with water in an ultrasonic mixer with polylysine added at 14.5% by weight of MSF. Then a HA polymer solution in water was added such that polylysine to HA ratio was at 25:1 on a weight basis, and the coating suspension was further mixed on ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 5.0:1.8:2.3 ratio on wt. basis with SA at 13% by weight of MSF. The coated catheters were P/F and packaged in a foil pack and were subjected to electron beam sterilization radiation at 25 kGY+/−1 kGY dose. Post sterilized DCB were used for in-vitro tracking and dynamic tissue adhesion test methodologies as described above. Residual drug content post in-vitro tracking was measured at 38.3% of label claim and dynamic tissue adhesion was measured at 40.1% of label claim drug.

### Example VI-32. Lot PLH-31

A batch of microspheres with mean diameter (D50) of 2.79 μm and Sirolimus drug content at 29% was mixed with water in an ultrasonic mixer with polylysine added at 17.7% by weight of MSF. Then a HA polymer solution in water was added such that polylysine to HA ratio was at 30:1 on a weight basis, and the coating suspension was further mixed on ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 5.0:1.5:1.94 ratio on a weight basis with SA at 13% by weight of MSF. The coated catheters were P/F and packaged in a foil pack and were subjected to electron beam sterilization radiation at 25 kGY+/−1 kGY dose. Post sterilized DCB were used for in-vitro tracking and dynamic tissue adhesion test methodologies as described above. Residual drug content post in-vitro tracking was measured at 38.4% of label claim and dynamic tissue adhesion was measured at 45.1% of label claim drug.

### Example VI-33. Lot PLH32

A batch of microspheres with mean diameter (D50) of 2.79 μm and Sirolimus drug content at 29% was mixed with water in an ultrasonic mixer with polylysine added at 17.7% by weight of MSF. Then a HA polymer solution in water was added such that polylysine to HA ratio was at 30:1 on a weight basis, and the coating suspension was further mixed on ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 5.0:1.8:3.3 ratio on a weight basis with SA at 10.8% by weight of MSF. The coated catheters were P/F and packaged in a foil pack and were subjected to electron beam sterilization radiation at 25 kGY+/−1 kGY dose. Post sterilized DCB were used for in-vitro tracking and dynamic tissue adhesion test methodologies as described above. Residual drug content post in-vitro tracking was measured at 34.3% of label claim and dynamic tissue adhesion was measured at 36.6% of label claim drug.

### Example VI-34. Lot 2LYL-16

A batch of microspheres with mean diameter (D50) of 2.74 μm and Sirolimus drug content at 27.3% was mixed with water in an ultrasonic mixer and the coating suspension was coated on 3×30, 3.5×30 and 5×40 mm balloon catheters targeting Sirolimus drug content of 1.8 μg/mm²+/−10%. These catheters were then coated with cyclohexane-based coating solutions containing POPC+BHT at 1:5:1 ratio on wt. basis with POPC at 10% by weight of MSF. The coated catheters were P/F and packaged in a foil pack and were subjected to electron beam sterilization radiation at 25 kGY+/−1 kGY dose. Post sterilized DCB were used for in-vitro tracking, dynamic tissue adhesion testing and in-vivo pk in porcine coronary arteries using the methodologies described above. Residual drug content post in-vitro tracking was measured at 35.4% of label claim and dynamic tissue adhesion was measured at 2.9% of label claim drug. Average 7 and 28 day in-vivo porcine coronary artery pk were measured as 0.33 & 0.22 μg/g respectively.

### Example VI-35. Lot 2LYL-7

A batch of microspheres with mean diameter (D50) of 2.18 μm and Sirolimus drug content at 29.1% was mixed with water in an ultrasonic mixer and the coating suspension was coated on 3×30 & 3.5×30 mm balloon catheters targeting Sirolimus drug content of 1.8 μg/mm²+/−10%. These catheters were then coated with cyclohexane-based coating solutions containing POPC:DOPC:DLPC:C6:BHT at 1:1:0.26:1.73:1.71 ratio on wt. basis with POPC at 5% by weight of MSF. C6 was 1,2-dihexanoyl-sn-glycero-3-phosphocholine. The coated catheters were P/F and packaged in a foil pack and were subjected to electron beam sterilization radiation at 25 kGY+/−1 kGY dose. Post sterilized DCB were used for in-vivo pk in porcine coronary arteries using the methodologies described above. Average 7 and 28 day in-vivo porcine coronary artery pk were measured as 0.147 μg/g and 0.038 μg/g, respectively.

### Example VI-36. Lot 2LYLB-04

A batch of microspheres with mean diameter (D50) of 0.98 μm and Sirolimus drug content at 63.2% was mixed with 60% BHT (by weight of microsphere) in heptane in an ultrasonic mixer and the coating suspension was coated on 3×30 & 3.5×30 mm balloon catheters targeting Sirolimus drug content of 1.8 μg/mm²+/−10%. These catheters were then coated with cyclohexane-based coating solutions containing POPC:DOPC:DLPC:C6 at 1:1:0.17:2.1 ratio on a weight basis with POPC at 14% by weight of MSF. C6 was 1,2-dihexanoyl-sn-glycero-3-phosphocholine. The coated catheters were P/F and packaged in a foil pack and were subjected to electron beam sterilization radiation at 25 kGY+/−1 kGY dose. Post sterilized DCB were used for in-vivo pk in porcine coronary arteries using the methodologies described above. Average 7 and 28 day in-vivo porcine coronary artery pk were measured as 0.118 & 0.074 μg/g respectively.

### Example VI-37. Lot F98-7

A batch of microspheres with mean diameter (D50) of 1.43 μm and Sirolimus drug content at 38.2% was mixed in cyclohexane with POPC:DOPC:PEE:C7:BHT (wherein PEE is pentaerythritol ethoxylate) at a ratio 1:0.1:0.2:0.1:1 on a weight basis with POPC at 64% of microspheres by weight, in an ultrasonic mixer and the coating suspension was coated on 3×30 mm and 3.5×30 mm balloon catheters targeting Sirolimus drug content of 1.8 μg/mm²+/−10%. C7 was 1,2-diheptanoyl-SN-glycero-3-phosphocholine. The coated catheters were P/F and packaged in a foil pack and were subjected to electron beam sterilization radiation at 25 kGY+/−1 kGY dose. Post sterilized DCB were used for in-vivo pk in porcine coronary arteries using the methodologies described above. Average 7 and 28 day in-vivo porcine coronary artery pk were measured as 0.72 μg/g and 0.085 μg/g, respectively.

### Example VI-38. Lot 2LYN-02

A batch of microspheres with mean diameter (D50) of 2.74 μm and Sirolimus drug content at 27.3% was mixed with water in an ultrasonic mixer and the coating suspension was coated on 3×30 mm, 3.5×30 mm, and 5×40 mm balloon catheters targeting Sirolimus drug content of 1.0 μg/mm²+/−10%. These catheters were then coated with cyclohexane-based coating solutions containing POPC+DPEPC+BHT+neat drug at 1.4:3.2:12.3:27.3 ratio on a weight basis with POPC at 1.4% by weight of MSF. Neat drug refers to sirolimus drug particles dispersed in cyclohexane with average particle size at 2 μm. Neat drug contributed another 1.0 μg/mm² dose density to equate to 2.0 μg/mm² total drug density. The coated catheters were P/F and packaged in a foil pack and were subjected to electron beam sterilization radiation at 25 kGY+/−1 kGY dose. Post sterilized DCB were used for in-vivo pk in porcine coronary arteries using the methodologies described above. Average 7 and 28 day in-vivo porcine coronary artery pk were measured as 0.584 μg/g and 0.117 μg/g, respectively.

### Example VI-39. Lot F65-2

A batch of microspheres with mean diameter (D50) of 1.06 μm and Sirolimus drug content at 37.7% was mixed in cyclohexane with POPC:C6:BHT at the ratio 1:0.2:2.13 on a weight basis with POPC at 50% of microspheres by weight, in an ultrasonic mixer and the coating suspension was coated on 3×30 mm and 3.5×30 mm balloon catheters targeting Sirolimus drug content of 2.125 μg/mm²+/−10%. C6 was 1,2-dihexanoyl-sn-glycero-3-phosphocholine. The coated catheters were P/F and packaged in a foil pack and were subjected to electron beam sterilization radiation at 25 kGY+/−1 kGY dose. Post sterilized DCB were used for in-vivo pk in porcine coronary arteries using the methodologies described above. Average 7 days in-vivo porcine coronary artery pk were measured as 13.22 μg/g.

### Example VI-40. Lot F66-3

A batch of microspheres with mean diameter (D50) of 1.06 μm and Sirolimus drug content at 37.7% was mixed in cyclohexane with POPC:PEE:BHT at the ratio 1:0.2:2.13 with POPC at 50% of microspheres by weight, in an ultrasonic mixer and the coating suspension was coated on 3×30 mm 3.5×30 mm balloon catheters targeting Sirolimus drug content of 2.125 μg/mm²+/−10%. The coated catheters were P/F and packaged in a foil pack and were subjected to electron beam sterilization radiation at 25 kGY+/−1 kGY dose. Post sterilized DCB were used for in-vivo pk in porcine coronary arteries using the methodologies described above. Average 7 days in-vivo porcine coronary artery pk were measured as 2.25 μg/g.

### Example VI-41. Lot PLH33

A batch of microspheres with mean diameter (D50) of 2.79 μm and Sirolimus drug content at 29% was mixed with water in an ultrasonic mixer with polylysine added at 18.2% by weight of MSF. Then a HA polymer solution in water was added such that polylysine to HA ratio was at 22:1 on a weight basis, and the coating suspension was further mixed on ultrasonic mixer. This coating suspension was coated on 3×20 mm and 5×40 mm balloon catheters targeting Sirolimus drug content of 1.8 μg/mm²+/−10%. These catheters were then coated with cyclohexane-based coating solutions containing SA+POPC+BHT at 4.07:1:1.29 ratio on a weight basis with SA at 11% by weight of MSF. The coated catheters were P/F and packaged in a foil pack and were subjected to electron beam sterilization radiation at 25 kGY+/−1 kGY dose. Post sterilized DCB were used for in-vitro tracking and dynamic tissue adhesion test methodologies as described above. Residual drug content post in-vitro tracking was measured at 44.25% of label claim and dynamic tissue adhesion was measured at 48.11% of label claim drug.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### Exemplary Aspects

The following exemplary aspects are provided, the numbering of which is not to be construed as designating levels of importance:

Aspect 1 provides a drug-releasing coating comprising:
polymer-encapsulated drug particles comprising
   a therapeutic agent,
   one or more polymers that encapsulate the therapeutic agent, and
   a first ionic or zwitterionic additive, wherein the first ionic or zwitterionic additive is in the polymer-encapsulated drug particles, coated on a surface of the polymer-encapsulated drug particles, or a combination thereof; and
a release matrix comprising a second ionic or zwitterionic additive.

Aspect 2 provides the drug-releasing coating of Aspect 1, wherein the first ionic or zwitterionic additive is in the polymer-encapsulated drug particles.

Aspect 3 provides the drug-releasing coating of any one of Aspects 1-2, wherein the first ionic or zwitterionic additive is coated on a surface of the polymer-encapsulated drug particles.

Aspect 4 provides the drug-releasing coating of any one of Aspects 1-3, wherein the first ionic or zwitterionic additive is in the polymer-encapsulated drug particles and coated on a surface of the polymer-encapsulated drug particles.

Aspect 5 provides the drug-releasing coating of any one of Aspects 1-4, wherein the polymer-encapsulated drug particle has a zeta potential of −2 to −40 or 2-40.

Aspect 6 provides the drug-releasing coating of any one of Aspects 1-5, wherein the therapeutic agent is sirolimus.

Aspect 7 provides the drug-releasing coating of any one of Aspects 1-6, wherein the polymer is at least one polymer chosen from polylactic acid (PL), polyglycolic acid (GA), a polylactic acid/polyglycolic acid copolymer (PLGA), polydioxanone, polycaprolactone, polyphosphazene, collagen, gelatin, chitosan, glycosoaminoglycans, and copolymers thereof.

Aspect 8 provides the drug-releasing coating of Aspect 7, wherein the polymer comprises PLGA.

Aspect 9 provides the drug-releasing coating of any one of Aspects 1-8, wherein the polymer-encapsulated drug particles have a mean diameter (D50) of 0.1 μm to 10 μm.

Aspect 10 provides the drug-releasing coating of any one of Aspects 1-9, wherein the polymer-encapsulated drug particles have a mean diameter (D50) of 0.5 μm to 5 μm.

Aspect 11 provides the drug-releasing coating of any one of Aspects 1-10, wherein the therapeutic agent is 10 wt % to 80 wt % of the polymer-encapsulated drug particles.

Aspect 12 provides the drug-releasing coating of any one of Aspects 1-11, wherein the therapeutic agent is 25 wt % to 65 wt % of the polymer-encapsulated drug particles.

Aspect 13 provides the drug-releasing coating of any one of Aspects 1-12, wherein the first ionic or zwitterionic additive comprises a charged polymer, a charged lipid, a phospholipid, a phosphocholine, a phosphatidylcholine, a phosphatidylethanolamine, a phosphatidylserine, a phosphatidylinositol, and combinations thereof.

Aspect 14 provides the drug-releasing coating of any one of Aspects 1-13, wherein the first ionic or zwitterionic additive comprises 1,2-disteroyl-sn-glycero-3-phosphatidylcholine (DSPC).

Aspect 15 provides the drug-releasing coating of any one of Aspects 1-14, wherein the first ionic or zwitterionic additive is 0.01 wt % to 50 wt % of the polymer-encapsulated drug particles.

Aspect 16 provides the drug-releasing coating of any one of Aspects 1-15, wherein the first ionic or zwitterionic additive is 0.1 wt % to 5 wt % of the polymer-encapsulated drug particles.

Aspect 17 provides the drug-releasing coating of any one of Aspects 1-16, wherein the first ionic or zwitterionic additive is 0.5 wt % to 2 wt % of the polymer-encapsulated drug particles.

Aspect 18 provides the drug-releasing coating of any one of Aspects 1-17, wherein the polymer-encapsulated drug particles comprise a phospholipid, a fatty acid component, an antioxidant, or a combination thereof.

Aspect 19 provides the drug-releasing coating of any one of Aspects 1-18, wherein the polymer-encapsulated drug particles are 1 wt % to 95 wt % of the drug-releasing coating.

Aspect 20 provides the drug-releasing coating of any one of Aspects 1-19, wherein the polymer-encapsulated drug particles are 25 wt % to 65 wt % of the drug-releasing coating.

Aspect 21 provides the drug-releasing coating of any one of Aspects 1-20, wherein the second ionic or zwitterionic additive comprises a charged polymer, a charged lipid, a phospholipid, a phosphocholine, a phosphatidylcholine, a phosphatidylethanolamine, a phosphatidylserine, a phosphatidylinositol, and combinations thereof.

Aspect 22 provides the drug-releasing coating of any one of Aspects 1-21, wherein the second ionic or zwitterionic additive comprises 1-palmitoyl-2-oleoyl-sn-glycero-3-phosphocholine (POPC), 1,2-dioleoyl-sn-glycero-3-phosphocholine (DOPC), 1,2-dipalmitoleoyl-sn-glycero-3-phosphocholine (DPEPC), 1,2-disteroyl-sn-glycero-3-phosphatidylcholine (DSPC), polylysine, polyarginine, hyaluronic acid (HA), or a combination thereof.

Aspect 23 provides the drug-releasing coating of any one of Aspects 1-22, wherein the second ionic or zwitterionic additive is 5 wt % to 99 wt % of the drug-releasing coating.

Aspect 24 provides the drug-releasing coating of any one of Aspects 1-23, wherein the second ionic or zwitterionic additive is 5 wt % to 65 wt % of the drug-releasing coating.

Aspect 25 provides the drug-releasing coating of any one of Aspects 1-24, wherein the second ionic or zwitterionic additive is present in the drug-releasing coating in an amount that is 0.01 wt % to 200 wt % of a total amount of the polymer-encapsulated drug particles in the drug-releasing coating.

Aspect 26 provides the drug-releasing coating of any one of Aspects 1-25, wherein the second ionic or zwitterionic additive is present in the drug-releasing coating in an amount that is 1 wt % to 150 wt % of a total amount of the polymer-encapsulated drug particles in the drug-releasing coating.

Aspect 27 provides the drug-releasing coating of any one of Aspects 1-26, wherein the second ionic or zwitterionic additive comprises a cationic polymer.

Aspect 28 provides the drug-releasing coating of Aspect 27, wherein the cationic polymer comprises polyethylenimine (PEI), polyallylamine, polypropylenimine, polyamidoamine dendrimer, cationic polyoxazoline, poly(beta-aminoester), PEG-PEI copolymer, PLGA-PEI copolymer, positively charged gelatin (e.g., base-treated gelatin), hydroxy-terminated poly(2-methyl-2-oxazoline), poly(2-ethyl-2-oxazoline), stearic acid-modified branched polyethylenimine, branched PEI-g-PEG, poly(1-vinylpyrrolidone-co-2-dimethylaminoethyl methacrylate), poly(1-vinylpyrrolidone)-graft-(1-triacontene), poly-L-lysine, poly-L-ornithine, poly(4-hydroxy-L-proline ester), polylysine, polyarginine, poly(N,N-dimethylaminoethyl methacrylate), cationic copolymer of dimethylaminoethyl methacrylate/butyl methacrylate/methyl methacrylate (e.g., Eudragit E), polycation-containing cyclodextrin, amino cyclodextrin or a derivative thereof, amino dextran, histone, protamine, cationized human serum albumin, aminopolysaccharide, chitosan, a peptide, polylysine, polyarginine, or a combination thereof.

Aspect 29 provides the drug-releasing coating of any one of Aspects 27-28, wherein the cationic polymer comprises polylysine.

Aspect 30 provides the drug-releasing coating of any one of Aspects 27-29, wherein the cationic polymer comprises polyarginine.

Aspect 31 provides the drug-releasing coating of any one of Aspects 27-30, wherein the release matrix comprises the cationic polymer in an amount that is 0.1% to 40% of a total weight of the polymer-encapsulated drug particles in the drug-releasing coating.

Aspect 32 provides the drug-releasing coating of any one of Aspects 27-31, wherein the release matrix comprises the cationic polymer in an amount that is 0.5% to 20% of a total weight of the polymer-encapsulated drug particles in the drug-releasing coating.

Aspect 33 provides the drug-releasing coating of any one of Aspects 1-32, wherein the second ionic or zwitterionic additive comprises an anionic polymer.

Aspect 34 provides the drug-releasing coating of Aspect 33, wherein the release matrix comprises the anionic polymer in an amount that is 0.1% to 10% of a total weight of the polymer-encapsulated drug particles in the drug-releasing coating.

Aspect 35 provides the drug-releasing coating of any one of Aspects 33-34, wherein the anionic polymer comprises hyaluronic acid (HA).

Aspect 36 provides the drug-releasing coating of any one of Aspects 1-35, wherein the second ionic or zwitterionic additive comprises a cationic polymer and an anionic polymer that are present in a ratio ranging from 1:1 to 60:1.

Aspect 37 provides the drug-releasing coating of any one of Aspects 1-36, wherein the second ionic or zwitterionic additive comprises a cationic polymer and an anionic polymer that are present in a ratio ranging from 2:1 to 30:1.

Aspect 38 provides the drug-releasing coating of any one of Aspects 1-37, wherein the release matrix comprises a phospholipid, a fatty acid component, an antioxidant, or a combination thereof.

Aspect 39 provides the drug-releasing coating of any one of Aspects 1-38, wherein the release matrix comprises an antioxidant.

Aspect 40 provides the drug-releasing coating of Aspect 39, wherein the antioxidant is present in the release matrix in an amount that is 0.1% to 150% of a total weight of the polymer-encapsulated drug particles in the drug-releasing coating.

Aspect 41 provides the drug-releasing coating of any one of Aspects 39-40, wherein the antioxidant is present in the release matrix in an amount that is 1% to 100% of a total weight of the polymer-encapsulated drug particles in the drug-releasing coating.

Aspect 42 provides the drug-releasing coating of any one of Aspects 39-41, wherein the antioxidant comprises BHT.

Aspect 43 provides the drug-releasing coating of any one of Aspects 1-42, wherein the release matrix comprises POPC, DOPC, PEE, a C6 or C7 fatty acid component (e.g., dihexanoyl-sn-glycero-3-phosphocholine or 1,2-diheptanoyl-sn-glycero-3-phosphocholine), and BHT.

Aspect 44 provides the drug-releasing coating of any one of Aspects 1-43, wherein the release matrix comprises POPC, DOPC, PEE, a C6 or C7 fatty acid component (e.g., dihexanoyl-sn-glycero-3-phosphocholine or 1,2-diheptanoyl-sn-glycero-3-phosphocholine), and BHT in a ratio of 0.5-2:0.01-0.3:0.1-0.5:0.01-0.3:0.5-2.

Aspect 45 provides the drug-releasing coating of any one of Aspects 1-44, wherein the release matrix comprises POPC, a C6 fatty acid component (e.g., dihexanoyl-sn-glycero-3-phosphocholine), and BHT.

Aspect 46 provides the drug-releasing coating of any one of Aspects 1-45, wherein the release matrix comprises POPC, a C6 fatty acid component (e.g., dihexanoyl-sn-glycero-3-phosphocholine), and BHT in a ratio of 0.5-2:0.1-1:1-5.

Aspect 47 provides the drug-releasing coating of any one of Aspects 1-46, further comprising a topcoat layer.

Aspect 48 provides the drug-releasing coating of Aspect 47, wherein the topcoat layer comprises a third ionic or zwitterionic additive.

Aspect 49 provides the drug-releasing coating of Aspect 48, wherein the third ionic or zwitterionic additive comprises a charged polymer, a charged lipid, a phospholipid, a phosphocholine, a phosphatidylcholine, a phosphatidylethanolamine, a phosphatidylserine, a phosphatidylinositol, and combinations thereof.

Aspect 50 provides the drug-releasing coating of any one of Aspects 48-49, wherein the third ionic or zwitterionic additive comprises 1-palmitoyl-2-oleoyl-sn-glycero-3-phosphocholine (POPC), 1,2-dioleoyl-sn-glycero-3-phosphocholine (DOPC), 1,2-dilauroyl-sn-glycero-3-phosphocholine (DLPC), 1,2-dipalmitoleoyl-sn-glycero-3-phosphocholine (DPEPC), 1,2-disteroyl-sn-glycero-3-phosphatidylcholine (DSPC), steric acid, palmitic acid, hexanoic acid, heptanoic acid, or a combination thereof.

Aspect 51 provides the drug-releasing coating of any one of Aspects 48-50, wherein the third ionic or zwitterionic additive is 10 wt % to 100 wt % of the topcoat layer.

Aspect 52 provides the drug-releasing coating of any one of Aspects 48-51, wherein the third ionic or zwitterionic additive is 65 wt % to 95 wt % of the topcoat layer.

Aspect 53 provides the drug-releasing coating of any one of Aspects 48-52, wherein the third ionic or zwitterionic additive is present in the topcoat layer in an amount that is 1% to 200% of a total weight of the polymer-encapsulated drug particles in the drug-releasing coating.

Aspect 54 provides the drug-releasing coating of any one of Aspects 48-53, wherein the third ionic or zwitterionic additive is present in the topcoat layer in an amount that is 3% to 150% of a total weight of the polymer-encapsulated drug particles in the drug-releasing coating.

Aspect 55 provides the drug-releasing coating of any one of Aspects 48-54, wherein the topcoat layer comprises a phospholipid, a fatty acid component, an antioxidant, or a combination thereof.

Aspect 56 provides the drug-releasing coating of any one of Aspects 48-55, wherein the third ionic or zwitterionic additive comprises at least one phospholipid.

Aspect 57 provides the drug-releasing coating of Aspect 56, wherein topcoat layer comprises the one or more phospholipids in an amount that is 1% to 150% of a total weight of the polymer-encapsulated drug particles in the drug-releasing coating.

Aspect 58 provides the drug-releasing coating of any one of Aspects 56-57, wherein the topcoat layer comprises the one or more phospholipids in an amount that is 3% to 140% of a total weight of the polymer-encapsulated drug particles in the drug-releasing coating.

Aspect 59 provides the drug-releasing coating of any one of Aspects 56-58, wherein the at least one phospholipid is chosen from 1-palmitoyl-2-oleoyl-sn-glycero-3-phosphocholine (POPC), 1,2-dioleoyl-sn-glycero-3-phosphocholine (DOPC), 1,2-dilauroyl-sn-glycero-3-phosphocholine (DLPC), 1,2-dipalmitoleoyl-sn-glycero-3-phosphocholine (DPEPC), 1,2-disteroyl-sn-glycero-3-phosphatidylcholine (DSPC), and combinations thereof.

Aspect 60 provides the drug-releasing coating of Aspect 59, wherein the at least one phospholipid comprises 1-palmitoyl-2-oleoyl-sn-glycero-3-phosphocholine (POPC).

Aspect 61 provides the drug-releasing coating of Aspect 59, wherein the at least one phospholipid comprises 1,2-dioleoyl-sn-glycero-3-phosphocholine (DOPC).

Aspect 62 provides the drug-releasing coating of Aspect 59, wherein the at least one phospholipid comprises 1,2-dilauroyl-sn-glycero-3-phosphocholine (DLPC).

Aspect 63 provides the drug-releasing coating of Aspect 59, wherein the at least one phospholipid comprises 1,2-dipalmitoleoyl-sn-glycero-3-phosphocholine (DPEPC).

Aspect 64 provides the drug-releasing coating of any one of Aspects 56-58, wherein the at least one phospholipid comprises a phosphatidylethanolamine.

Aspect 65 provides the drug-releasing coating of any one of Aspects 48-64, wherein the third ionic or zwitterionic additive comprises at least one fatty acid component.

Aspect 66 provides the drug-releasing coating of Aspect 65, wherein the topcoat layer comprises the one or more fatty acid components in an amount that is 1% to 30% of a total weight of the polymer-encapsulated drug particles in the drug-releasing coating.

Aspect 67 provides the drug-releasing coating of any one of Aspects 65-66, wherein the topcoat layer comprises the one or more fatty acid components in an amount that is 2% to 20% of a total weight of the polymer-encapsulated drug particles in the drug-releasing coating.

Aspect 68 provides the drug-releasing coating of any one of Aspects 65-67, wherein the at least one fatty acid component comprises a C6-C20 fatty acid component.

Aspect 69 provides the drug-releasing coating of any one of Aspects 65-68, wherein the at least one fatty acid component is chosen from stearic acid 50, a C6 fatty acid component (e.g., dihexanoyl-sn-glycero-3-phosphocholine), a C7 fatty acid component (e.g., 1,2-diheptanoyl-sn-glycero-3-phosphocholine), and combinations thereof.

Aspect 70 provides the drug-releasing coating of Aspect 69, wherein the at least one fatty acid component comprises stearic acid 50.

Aspect 71 provides the drug-releasing coating of Aspect 70, wherein the stearic acid 50 comprises a blend of stearic and palmitic acids.

Aspect 72 provides the drug-releasing coating of any one of Aspects 69-71, wherein the at least one fatty acid component comprises a C6 fatty acid component (e.g., dihexanoyl-sn-glycero-3-phosphocholine).

Aspect 73 provides the drug-releasing coating of any one of Aspects 69-72, wherein the at least one fatty acid component comprises a C7 fatty acid component (e.g., 1,2-diheptanoyl-sn-glycero-3-phosphocholine).

Aspect 74 provides the drug-releasing coating of any one of Aspects 47-73, wherein the topcoat layer comprises BHT.

Aspect 75 provides the drug-releasing coating of Aspect 74, wherein the BHT is present in the topcoat in an amount that is 0.1% to 120% of a total weight of the polymer-encapsulated drug particles in the drug-releasing coating.

Aspect 76 provides the drug-releasing coating of any one of Aspects 74-75, wherein the BHT is present in the topcoat in an amount that is 1% to 20% of a total weight of the polymer-encapsulated drug particles in the drug-releasing coating.

Aspect 77 provides the drug-releasing coating of any one of Aspects 47-76, wherein the topcoat layer comprises neat drug particles.

Aspect 78 provides the drug-releasing coating of Aspect 77, wherein the neat drug particles comprise sirolimus particles having an average particle size of 0.5 μm to 10 μm.

Aspect 79 provides the drug-releasing coating of any one of Aspects 77-78, wherein the neat drug particles comprise sirolimus particles having an average particle size of 1 μm to 3 μm.

Aspect 80 provides the drug-releasing coating of any one of Aspects 47-79, wherein the topcoat layer comprises POPC and BHT in a weight ratio of 0.1:1 to 10:1.

Aspect 81 provides the drug-releasing coating of any one of Aspects 47-80, wherein the topcoat layer comprises POPC and BHT in a weight ratio of 0.5:1 to 2:1.

Aspect 82 provides the drug-releasing coating of any one of Aspects 47-81, wherein the topcoat layer comprises stearic acid 50 and POPC in a weight ratio ranging from 1:1 to 20:1.

Aspect 83 provides the drug-releasing coating of any one of Aspects 47-82, wherein the topcoat layer comprises stearic acid 50, POPC, and BHT in a weight ratio of 1-10:0.5-8:0.5-4.

Aspect 84 provides the drug-releasing coating of any one of Aspects 47-83, wherein the topcoat layer comprises stearic acid 50, POPC, and BHT in a weight ratio of 2-7:1-4:1-2.

Aspect 85 provides the drug-releasing coating of any one of Aspects 47-84, wherein the topcoat layer comprises POPC, DOPC, DLPC, C6 (e.g., dihexanoyl-sn-glycero-3-phosphocholine), and BHT in a weight ratio of 0.5-2:0.5-2:0.1-0.5:1-3:1-3.

Aspect 86 provides the drug-releasing coating of any one of Aspects 47-85, wherein the topcoat layer comprises POPC, DPEPC, BHT, and neat drug in a weight ratio of 1-2:1-5:5-15:20-40.

Aspect 87 provides the drug-releasing coating of any one of Aspects 47-86, wherein the topcoat layer is present in an amount that is 1% to 90% of a total weight of the polymer-encapsulated drug particles in the drug-releasing coating.

Aspect 88 provides the drug-releasing coating of any one of Aspects 47-87, wherein the topcoat layer is present in an amount that is 5% to 65% of a total weight of the polymer-encapsulated drug particles in the drug-releasing coating.

Aspect 89 provides a balloon catheter comprising: an elongated balloon; and a coating layer overlying an exterior surface of the balloon, the coating layer comprising the drug-releasing coating of any one of Aspects 1-88.

Aspect 90 provides the balloon catheter of Aspect 89, wherein the coating layer has a dose density of the therapeutic agent of 0.1 μg/mm² to 10 μg/mm².

Aspect 91 provides the balloon catheter of any one of Aspects 89-90, wherein the coating layer has a dose density of the therapeutic agent of 0.5 μg/mm² to 5 μg/mm².

Aspect 92 provides the balloon catheter of any one of Aspects 89-91, wherein the coating layer has a zeta potential of −50 mV to 100 mV.

Aspect 93 provides the balloon catheter of any one of Aspects 89-92, wherein the coating layer has a zeta potential of 15 mV to 100 mV.

Aspect 94 provides the balloon catheter of any one of Aspects 89-93, wherein the coating layer has a zeta potential of 25 mV to 80 mV.

Aspect 95 provides the balloon catheter of any one of Aspects 89-94, having a diameter of 0.5 mm to 20 mm.

Aspect 96 provides the balloon catheter of any one of Aspects 89-95, having a diameter of 1 mm to 5 mm.

Aspect 97 provides the balloon catheter of any one of Aspects 89-96, having a length of 5 mm to 200 mm.

Aspect 98 provides the balloon catheter of any one of Aspects 89-97, having a length of 10 mm to 50 mm.

Aspect 99 provides a method of making the drug-releasing coating of any one of Aspects 1-88, the method comprising:
dispersing polymer-encapsulated drug particles in water using ultrasonic mixing;
adding a second ionic or zwitterionic additive to the dispersion; and
applying the dispersion to a balloon catheter.

Aspect 100 provides the method of Aspect 99, further comprising applying a topcoat layer comprising at least one phospholipid.

Aspect 101 provides the method of Aspect 100, wherein the topcoat layer is applied using cyclohexane as a solvent.

Aspect 102 provides the method of any one of Aspects 100-101, wherein the topcoat layer is applied using heptane as a solvent.

Aspect 103 provides the method of any one of Aspects 99-102, further comprising sterilizing the coated balloon catheter using electron beam radiation.

Aspect 104 provides the method of Aspect 103, wherein the electron beam radiation is applied at a dose of 10 kGy to 100 kGy.

Aspect 105 provides the method of any one of Aspects 103-104, wherein the electron beam radiation is applied at a dose of 15 kGy to 40 kGy.

Aspect 106 provides a method for treating a vascular condition, the method comprising:
providing the balloon catheter of any one of Aspects 89-98;
inserting the balloon catheter into a target site;
inflating the balloon catheter to contact the coating layer with tissue at the target site; and
deflating and removing the balloon catheter.

Aspect 107 provides the method of Aspect 106, wherein the tissue at the target site retains 1% to 100% of the therapeutic agent in the coating layer.

Aspect 108 provides the method of any one of Aspects 106-107, wherein the tissue at the target site retains 20% to 90% of the therapeutic agent in the coating layer.

Aspect 109 provides the method of any one of Aspects 106-108, wherein the tissue at the target site retains 50% to 90% of the therapeutic agent in the coating layer.

Aspect 110 provides a drug-releasing coating comprising:
polymer-encapsulated drug particles comprising sirolimus and PLGA polymer; and
a release matrix comprising polylysine and/or polyarginine in an amount that is 0.5% to 20% of a total weight of the polymer-encapsulated drug particles in the drug-releasing coating.

Aspect 111 provides the drug-releasing coating of Aspect 110, further comprising hyaluronic acid in a polylysine and/or polyarginine to hyaluronic acid ratio of 1:1 to 60:1.

Aspect 112 provides the drug-releasing coating of any one of Aspects 110-111, further comprising a topcoat layer comprising stearic acid 50 and POPC.

Aspect 113 provides a drug-releasing coating comprising:
polymer-encapsulated drug particles having a mean diameter (D50) of 0.5 μm to 5 μm and sirolimus drug content of 25 wt % to 65 wt %;
polylysine and/or polyarginine at 0.5% to 20% by weight of the polymer-encapsulated drug particles;
hyaluronic acid in a polylysine and/or polyarginine to hyaluronic acid ratio of 1:1 to 60:1; and
a topcoat layer comprising stearic acid 50, POPC, and BHT in a ratio of 2-7:1-4:1-2.

Aspect 114 provides a drug-releasing coating comprising:
polymer-encapsulated drug particles having a mean diameter (D50) of 0.5 μm to 5 μm and sirolimus drug content of 25 wt % to 65 wt %;
POPC, DOPC, PEE, C6 or C7 fatty acid component (e.g., dihexanoyl-sn-glycero-3-phosphocholine or 1,2-diheptanoyl-sn-glycero-3-phosphocholine), and BHT in a ratio of 0.5-2:0.01-0.3:0.1-0.5:0.01-0.3:0.5-2; and
wherein POPC is present at 50% to 80% of a total weight of the of the polymer-encapsulated drug particles in the drug-releasing coating.

Aspect 115 provides the drug-releasing coating of Aspect 114, applied to achieve a target dose density of 0.5 μg/mm² to 5 μg/mm².

Aspect 116 provides a drug-releasing coating comprising:
polymer-encapsulated drug particles having a mean diameter (D50) of 0.5 μm to 5 μm and sirolimus drug content of 25 wt % to 65 wt %;
POPC, C6 fatty acid component (e.g., dihexanoyl-sn-glycero-3-phosphocholine), and BHT in a ratio of 0.5-2:0.1-1:1-5; and
wherein POPC is present at 30% to 70% of a total weight of the polymer-encapsulated drug particles in the drug-releasing coating.

Aspect 117 provides the drug-releasing coating of Aspect 116, applied to achieve a target dose density of 0.5 μg/mm² to 5 μg/mm².

Aspect 118 provides a method of making polymer-encapsulated drug particles, the method comprising:
dissolving sirolimus, PLGA polymer, and a first ionic or zwitterionic additive in dichloromethane to form an oil phase;
dispersing the oil phase in a water phase containing polyvinyl alcohol using high-pressure microfluidic apparatus;
mixing the oil-water mixture with excess water for stabilization and solvent evaporation;
centrifuging to separate microspheres; and
lyophilizing to obtain dried microsphere powder.

Aspect 119 provides the method of Aspect 118, wherein the oil phase comprises dichloromethane solvent.

Aspect 120 provides the method of any one of Aspects 118-119, wherein the high-pressure microfluidic apparatus operates at pressures of 20,000 to 40,000 psi.

Aspect 121 provides the method of any one of Aspects 118-120, wherein the microspheres have a mean diameter (D50) of 1 μm to 5 μm.

Aspect 122 provides the method of any one of Aspects 118-121, further comprising optimizing particle size distribution to achieve narrow size distributions.

Aspect 123 provides a method of applying a drug-releasing coating, the method comprising:
dispersing polymer-encapsulated drug particles in water using ultrasonic mixing;
adding polylysine and/or polyarginine in an amount that is 0.5% to 20% of a total weight of the polymer-encapsulated drug particles;
optionally adding hyaluronic acid solution;
applying the coating suspension to a balloon catheter targeting a dose density of the drug of 0.5 μg/mm² to 5 μg/mm²; and
applying a topcoat layer using cyclohexane or heptane as solvent.

Aspect 124 provides the method of Aspect 123, wherein the ultrasonic mixing is performed using an ultrasonic mixing horn.

Aspect 125 provides the method of any one of Aspects 123-124, further comprising pleat and folding the coated balloon catheter.

Aspect 126 provides the method of any one of Aspects 123-125, further comprising sterilizing the balloon catheter using electron beam radiation at 10 kGy to 100 kGy.

Aspect 127 provides a drug-releasing coating system comprising:
an aqueous base layer comprising polymer-encapsulated drug particles, polylysine and/or polyarginine, and optionally hyaluronic acid; and
a non-aqueous topcoat layer comprising one or more phospholipids, one or more fatty acids, and one or more antioxidants dissolved in cyclohexane or heptane.

Aspect 128 provides the drug-releasing coating system of Aspect 127, wherein the base layer and topcoat layer provide controlled drug release and enhanced tissue adhesion through cationic charge.

Aspect 129 provides the drug-releasing coating system of any one of Aspects 127-128, designed to provide improved retention during catheter tracking through tortuous anatomy.

Aspect 130 provides the drug-releasing coating, balloon catheter, method, or drug-releasing coating system of any one or any combination of Aspects 1-129 optionally configured such that all elements or options recited are available to use or select from.

What is claimed is:

1. A drug-releasing coating comprising:
polymer-encapsulated drug particles comprising
a therapeutic agent,
one or more polymers that encapsulate the therapeutic agent, and
a first ionic or zwitterionic additive, wherein the first ionic or zwitterionic additive is in the polymer-encapsulated drug particles; and
a release matrix comprising a second ionic or zwitterionic additive.

2. The drug-releasing coating of claim 1, wherein the therapeutic agent comprises sirolimus, paclitaxel, or a combination thereof, and wherein the therapeutic agent is 10 wt % to 80 wt % of the polymer-encapsulated drug particles.

3. The drug-releasing coating of claim 1, wherein the polymer comprises a polylactic acid/polyglycolic acid copolymer (PLGA).

4. The drug-releasing coating of claim 1, wherein the polymer-encapsulated drug particles have a mean diameter (D50) of 0.1 μm to 10 km.

5. The drug-releasing coating of claim 1, wherein the first ionic or zwitterionic additive comprises 1,2-disteroyl-sn-glycero-3-phosphatidylcholine (DSPC), and wherein the first ionic or zwitterionic additive is 0.01 wt % to 50 wt % of the polymer-encapsulated drug particles.

6. The drug-releasing coating of claim 1, wherein the polymer-encapsulated drug particles are 25 wt % to 65 wt % of the drug-releasing coating.

7. The drug-releasing coating of claim 1, wherein the second ionic or zwitterionic additive comprises 1-palmitoyl-2-oleoyl-sn-glycero-3-phosphocholine (POPC), 1,2-dioleoyl-sn-glycero-3-phosphocholine (DOPC), 1,2-dipalmitoleoyl-sn-glycero-3-phosphocholine (DPEPC), 1,2-disteroyl-sn-glycero-3-phosphatidylcholine (DSPC), polylysine, polyarginine, hyaluronic acid (HA), or a combination thereof, wherein the second ionic or zwitterionic additive is 5 wt % to 99 wt % of the drug-releasing coating, and wherein the second ionic or zwitterionic additive is present in the drug-releasing coating in an amount that is 0.01 wt % to 200 wt % of a total amount of the polymer-encapsulated drug particles in the drug-releasing coating.

8. The drug-releasing coating of claim 1, wherein the second ionic or zwitterionic additive comprises a cationic polymer.

9. The drug-releasing coating of claim 8, wherein the cationic polymer comprises polylysine, polyarginine, or a combination thereof, and wherein the release matrix comprises the cationic polymer in an amount that is 0.1% to 40% of a total weight of the polymer-encapsulated drug particles in the drug-releasing coating.

10. The drug-releasing coating of claim 1, wherein the second ionic or zwitterionic additive comprises an anionic polymer, and wherein the release matrix comprises the anionic polymer in an amount that is 0.1% to 10% of a total weight of the polymer-encapsulated drug particles in the drug-releasing coating.

11. The drug-releasing coating of claim 10, wherein the anionic polymer comprises hyaluronic acid (HA).

12. The drug-releasing coating of claim 1, wherein the release matrix comprises an antioxidant, wherein the antioxidant is present in the release matrix in an amount that is 0.1% to 150% of a total weight of the polymer-encapsulated drug particles in the drug-releasing coating.

13. The drug-releasing coating of claim 12, wherein the antioxidant comprises BHT.

14. The drug-releasing coating of claim 1, further comprising a topcoat layer comprising a third ionic or zwitterionic additive comprising 1-palmitoyl-2-oleoyl-sn-glycero-3-phosphocholine (POPC), 1,2-dioleoyl-sn-glycero-3-phosphocholine (DOPC), 1,2-dilauroyl-sn-glycero-3-phosphocholine (DLPC), 1,2-dipalmitoleoyl-sn-glycero-3-phosphocholine (DPEPC), 1,2-disteroyl-sn-glycero-3-phosphatidylcholine (DSPC), steric acid, palmitic acid, hexanoic acid, heptanoic acid, or a combination thereof, wherein the third ionic or zwitterionic additive is 10 wt % to 100 wt % of the topcoat layer, and wherein the third ionic or zwitterionic additive is present in the topcoat layer in an amount that is 1% to 200% of a total weight of the polymer-encapsulated drug particles in the drug-releasing coating.

15. The drug-releasing coating of claim 14, wherein the third ionic or zwitterionic additive comprises at least one fatty acid component in an amount that is 1% to 30% of a total weight of the polymer-encapsulated drug particles in the drug-releasing coating, and wherein the at least one fatty acid component comprises a 1,2-(C6-C20 fatty acid ester)-sn-glycero-3-phosphocholine.

16. The drug-releasing coating of claim 14, wherein the topcoat layer comprises BHT that is present in the topcoat layer in an amount that is 0.1% to 120% of a total weight of the polymer-encapsulated drug particles in the drug-releasing coating.

17. The drug-releasing coating of claim 14, wherein the topcoat layer comprises neat drug particles comprising sirolimus and/or paclitaxel, wherein the neat drug particles have an average particle size of 0.5 μm to 10 μm.

18. The drug-releasing coating of claim 14, wherein the topcoat layer is present in an amount that is 1% to 90% of a total weight of the polymer-encapsulated drug particles in the drug-releasing coating.

19. A balloon catheter comprising:
an elongated balloon; and
a coating layer overlying an exterior surface of the balloon, the coating layer comprising the drug-releasing coating of claim 1.

20. A method for treating a target site, the method comprising:
inserting a medical device comprising the drug-releasing coating of claim 1 thereon into a target site;
contacting the coating layer with tissue at the target site; and
removing the medical device from the target site.
